(19) United States
(12) Patent Application Publication
(10) Pub. No.: US 2004/0185456 A1
(43) Pub. Date: Sep. 23, 2004

(54) Nucleic and proteinic acids corresponding to human gene ABC1

(75) Inventors: Patrice Denefle, Saint Maur (FR); Marie-Francoise Rosier-Montus, Antony (FR); Isabelle Arnould-Reguigne, Chennevieres sur Marne (FR); Catherine Prades, Thiais (FR); Laurent Naudin, Etampes (FR); Cendrine Lemoine, Massy (FR); Nicolas Duverger, Paris (FR); Stephan Rust, Munster (DE); Gerd Assmann, Munster (DE); Harald Funke, Munster (DE); H. Bryan Brewer, Potomac, MD (US)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: Aventis Pharma S.A., Antony (FR)

(21) Appl. No.: 10/465,498

(22) Filed: Jun. 18, 2003

Related U.S. Application Data
(63) Continuation of application No. 10/269,780, filed on Oct. 11, 2002, now abandoned.
Application No. 10/269,780 is a continuation of application No. 10/018,714, now abandoned.
Application No. 10/018,714 is a 371 of international application No. PCT/FR00/01595, filed on Jun. 8, 2000.
(60) Provisional application No. 60/147,128, filed on Aug. 4, 1999.

(30) Foreign Application Priority Data
Jun. 17, 1999 (FR) 99 07684

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The invention relates to nucleic acids corresponding to different exons and introns of gene ABC1 which is shown to be a gene causing pathologies linked to cholesterol metabolism dysfunction causing diseases such as atherosclerosis, more particularly perturbation of reverse cholesterol transport and more particularly the FHD's such as Tangier Disease

The present invention relates to nucleic acids corresponding to the various exons and introns of the ABC1 gene, for which it is now demonstrated that it is a causal gene for pathologies linked to a cholesterol metabolism dysfunction inducing diseases such as atherosclerosis, more particularly disruption in the reverse transport of cholesterol, and more particularly familial HDL deficiencies (FHD), such as Tangier disease. The invention also relates to means for the detection of polymorphisms in general, and of mutations in particular, in the ABC1 gene or in the corresponding protein produced by the allelic form of the ABC1 gene. The invention also provides pharmaceutical compositions comprising a nucleic acid containing the coding region of the ABC1 gene and pharmaceutical compositions containing the ABC1 protein intended for the treatment of diseases linked to a deficiency in the reverse transport of cholesterol, such as Tangier disease. The invention also provides methods for screening small molecules acting on the ABC1 protein which may by itself constitute products acting on the reverse transport of cholesterol and as such may make it possible to effectively combat atherosclerosis from a therapeutic point of view.

High-density lipoproteins (HDL) are one of the four major classes of lipoproteins circulating in blood plasma.

These lipoproteins are involved in various metabolic pathways such as lipid transport, the formation of bile acids, steroidogenesis, cell proliferation and, in addition, interfere with the plasma proteinase systems.

HDLs are perfect free cholesterol acceptors and, in combination with the cholesterol ester transfer proteins (CETP), lipoprotein lipase (LPL), hepatic lipase (HL) and lecithin:cholesterol acyltransferase (LCAT), play a major role in the reverse transport of cholesterol, that is to say the transport of excess cholesterol in the peripheral cells to the liver for its elimination from the body in the form of bile acid.

It has been demonstrated that the HDLs play a central role in the transport of cholesterol from the peripheral tissues to the liver.

Various diseases linked to an HDL deficiency have been described, including Tangier disease, HOL deficiency and LCAT deficiency.

The deficiency involved in Tangier disease is linked to a cellular defect in the translocation of cellular cholesterol which cause a degradation of the HDLs. Nevertheless, for Tangier disease, the exact nature of the defect has not yet been precisely defined.

In Tangier disease, this cellular defect leads to a disruption in the lipoprotein metabolism. The HDL particles not incorporating cholesterol from the peripheral cells and not being able to be metabolized correctly, are rapidly eliminated from the body. The plasma HDL concentration in these patients is therefore extremely reduced and the HDLs no longer ensure the return of cholesterol to the liver. This cholesterol accumulates in these peripheral cells and cause characteristic clinical manifestations such as the formation of orange-colored tonsils. Furthermore, other lipoprotein disruptions such as overproduction of triglycerides as well as increased synthesis and intracellular catabolism of phospholipids are observed.

Tangier disease, whose symptoms have been described above, is classified among the familial conditions linked to the metabolism of HDLs which are the ones most commonly detected in patients affected by coronary diseases.

Numerous studies have shown that a reduced level of HDL cholesterol is an excellent risk factor which makes it possible to detect a coronary condition.

In this context, syndromes linked to HDL deficiencies have been of increasing interest for the past decade because they make it possible to increase understanding of the role of HDLs in atherogenesis.

Several mutations in the apo A-I gene have been characterized. These mutations are rare and may lead to a lack of production of apo A-I.

Mutations in the genes encoding lipoprotein lipase (LPL) or its activator apo C-II are associated with severe hypertriglyceridemias and substantially reduced HDL-c levels.

Mutations in the gene encoding the enzyme lecithin;cholesterol acyltransferase (LCAT) are also associated with a severe HDL deficiency.

Furthermore, dysfunctions in the reverse transport of cholesterol may be induced by physiological deficiencies affecting one or more of the steps in the transport of stored cholesterol, from the intracellular vesicles to the membrane surface where it is accepted by the HDLs.

An increasing need therefore exists in the state of the art to identify genes involved in any of the steps in the metabolism of cholesterol and/or lipoproteins, and in particular genes associated with dysfunctions in the reverse transport of cholesterol from the peripheral cells to the liver.

Recently, a study was carried out of the segregation of different allelic forms of 343 microsatellite markers distributed over the entire genome and distant from each other by 10.3 cM on average.

The linkage study was carried out on a family which has been well characterized over eleven generations, in which many members are affected by Tangier disease, the family comprising five consanguineous lines.

This study made it possible to identify a region located in the 9q31 locus of human chromosome 9 which is statistically associated with the condition (Rust S. et al., Nature Genetics, vol. 20, September 1998, pages 96-98).

However, the study by RUST et al. only defines a wide region of the genome whose impairments are likely to be associated with Tangier disease. It is simply specified that the relevant 9q31-34 region contains ESTs but no known gene.

It has since been shown according to the invention that a region of about 1 cM situated in the 9q31 locus in humans was generally associated with familial HDL deficiencies.

More precisely, it has been shown that a gene encoding a protein of the family of ABC transporters, which is located precisely in the region of 1 cM of the 9q31 locus, was involved in pathologies linked to a deficiency in the reverse transport of cholesterol.

More particularly, it has been shown according to the invention that the gene encoding the ABC-1 transporter was mutated in patients impaired in the reverse transport of cholesterol, and most particularly in patients suffering from Tangier disease.

The ABC (“ATP-binding cassette”) transport proteins constitute a family of proteins which are extremely well conserved during evolution, from bacterial to humans.

The ABC transport proteins are involved in the membrane transport of various substrates, for example ions, amino acids, peptides, sugars, vitamins or steroid hormones.

The characterization of the complete amino acid sequence of some ABC transporters has made it possible to determine that these proteins had a common general structure, in particular two nucleotide binding folds (NBF) with Walker A and B type units as well as two transmembrane domains, each of the transmembrane domains consisting of six helices. The specificity of the ABC transporters for the various transported molecules appears to be determined by the structure of the transmembrane domains, whereas the energy necessary for the transport activity is provided by the degradation of ATP at the level of the NBF fold.

Several ABC transport proteins which have been identified in humans have been associated with various diseases.

For example, cystic fibrosis is caused by mutations in the CFTR (cystic fibrosis transmembrane conductance regulator) gene.

Moreover, some multiple drug resistance phenotypes in tumor cells have been associated with mutations in the gene encoding the MDR (multi-drug resistance) protein, which also has an ABC transporter structure.

Other ABC transporters have been associated with neuronal and tumor conditions (patent U.S. Pat. No. 5,858,719) or potentially involved in diseases caused by impairment of the homeostasis of metals, such as the ABC-3 protein.

Likewise, another transport ABC, designated PFIC2, appears to involve in a progressive familial intrahepatic cholestasia form, this protein being potentially responsible, in humans, for the export of bile salts.

In 1994, a cDNA encoding a new mouse ABC transporter was identified and designated ABC1 (Luciani et al., 1994). This protein is characteristic of the ABC transporters in that it has a symmetrical structure comprising two transmembrane domains linked to a highly hydrophobic segment and two NBF units.

In humans, a partial cDNA comprising the entire open reading frame of the human ABC1 transporter has been identified (Langmann et al., 1999).

It has also been shown that the gene encoding the human ABC1 protein is expressed in various tissues, and more particularly at high levels in the placenta, the liver, the lungs, the adrenal glands as well as the fetal tissues.

These authors have also shown that the expression of the gene encoding the human ABC1 protein was induced during the differentiation of the monocytes into macrophages in vitro. Furthermore, the expression of the gene encoding the ABC1 protein is increased when the human macrophages are incubated in the presence of acetylated low-density lipoproteins (AcLDLs).

However, the exact role of the human ABC1 protein in the lipid transport system is completely unknown. It is simply assumed that the ABC1 protein has a translocase activity for phospholipids.

It has now been shown, according to the invention, that patients suffering from Tangier disease had a mutated ABC1 gene. Several mutations distributed in different exons of the ABC1 gene have been identified in the genome of various patients, in particular patients suffering from a severe form of the disease associated with coronary disorders. Moreover, various polymorphisms have been found both in the exons and in the introns of the ABC1 gene in patients suffering from the mildest forms of the disease, indicating that these patients carry particular alleles of the gene, distinct from the “wild-type” allele(s). Such alleles, partly characterizable by these polymorphisms, are moreover likely to contain substitutions, additions or deletions of nucleotides in the noncoding regions located respectively on the 5′ side of the first exon or alternatively on the 3′ side of the last exon of the gene, in particular in the regulatory regions, for example in the promoter sequences or alternatively in the enhancer sequences, of the type which induces defects—increase or decrease—in the synthesis of the ABC1 polypeptide.

A first particular mutation has thus been identified in a patient suffering from Tangier disease, in the ABC-1 gene, which is located in exon 13, and which consists of a substitution of a nucleotide causing the introduction of a codon for premature termination of translation into the open reading frame, leading to the synthesis of a truncated polypeptide comprising about a quarter of the amino acid sequence of the polypeptide synthesized in patients not affected by Tangier disease.

A second particular mutation in the ABC1 gene has been found, which consists in an insertion of a fragment of 100 nucleotides into exon 12, leading to the synthesis of a polypeptide which is abnormal in that it contains a deletion of 6 residues and an insertion of 38 amino acids, at position 468 of the sequence of the protein.

It has, in addition, been confirmed according to the invention that the ABC1 gene was positively regulated by the acetylated low-density lipoproteins (AcLDLs).

### General Definitions

The term “isolated” for the purposes of the present invention designates a biological material (nucleic acid or protein) which has been removed from its original environment (the environment in which it is naturally present).

For example, a polynucleotide present in the natural state in a plant or an animal is not isolated. The same polynucleotide separated from the adjacent nucleic acids in which it is naturally inserted in the genome of the plant or animal is considered as being “isolated”.

Such a polynucleotide may be included in a vector and/or such a polynucleotide may be included in a composition and remains nevertheless in the isolated state because of the fact that the vector or the composition does not constitute its natural environment.

The term “purified” does not require the material to be present in a form exhibiting absolute purity, exclusive of the presence of other compounds. It is rather a relative definition.

A polynucleotide is in the “purified” state after purification of the starting material or of the natural material by at least one order of magnitude, preferably 2 or 3 and preferably 4 or 5 orders of magnitude.

For the purposes of the present description, the expression “nucleotide sequence” may be used to designate either a polynucleotide or a nucleic acid. The expression “nucleotide sequence” covers the genetic material itself and is therefore not restricted to the information relating to its sequence.

The terms “nucleic acid”, “polynucleotide”, “oligonucleotide” or “nucleotide sequence” cover RNA, DNA or cDNA sequences or alternatively RNA/DNA hybrid sequences of more than one nucleotide, either in the single-chain form or in the duplex form.

The term “nucleotide” designates both the natural nucleotides (A, T, G, C) as well as the modified nucleotides which comprise at least one modification such as (1) an analog of a purine, (2) an analog of a pyrimidine, or (3) an analogous sugar, examples of such modified nucleotides being described, for example, in the PCT application No. WO 95/04064.

For the purposes of the present invention, a first polynucleotide is considered as being “complementary” to a second polynucleotide when each base of the first nucleotide is paired with the complementary base of the second polynucleotide whose orientation is reversed. The complementary bases are A and T (or A and U), or C and G.

“Variant” of a nucleic acid according to the invention will be understood to mean a nucleic acid which differs by one or more bases relative to the reference polynucleotide. A variant nucleic acid may be of natural origin, such as an allelic variant which exists naturally, or it may also be a nonnatural variant obtained, for example, by mutagenic techniques.

In general, the differences between the reference nucleic acid and the variant nucleic acid are small such that the nucleotide sequences of the reference nucleic acid and of the variant nucleic acid are very similar and, In many regions, identical. The nucleotide modifications present in a variant nucleic acid may be silent, which means that they do not alter the amino acid sequences encoded by said variant nucleic acid.

However, the changes in nucleotides in a variant nucleic acid may also result in substitutions, additions or deletions in the polypeptide encoded by the variant nucleic acid in relation to the peptides encoded by the reference nucleic acid in addition, nucleotide modifications in the coding regions may produce conservative or nonconservative substitutions in the amino acid sequence.

Preferably, the variant nucleic acids according to the invention encode polypeptides which substantially conserve the same function or biological activity as the polypeptide of the reference nucleic acid or alternatively the capacity to be recognized by antibodies directed against the polypeptides encoded by the initial nucleic acid.

Some variant nucleic acids will thus encode mutated forms of the polypeptides whose systematic study will make it possible to deduce structure-activity relationships of the proteins in question. Knowledge of these variants in relation to the disease studied is essential since it makes it possible to understand the molecular cause of the pathology.

“Fragment” will be understood to mean a reference nucleic acid according to the invention, a nucleotide sequence of reduced length relative to the reference nucleic acid and comprising, over the common portion, a nucleotide sequence identical to the reference nucleic acid.

Such a nucleic acid “fragment” according to the invention may be, where appropriate, included in a larger polynucleotide of which it is a constituent.

Such fragments comprise, or alternatively consist of, oligonucleotides ranging in length from 8, 10, 12, 15, 18, 20 to 25, 30, 40, 50, 70, 80, 100, 200, 500, 1000 or 1500 consecutive nucleotides of a nucleic acid according to the invention.

“Variant” of a polypeptide according to the invention will be understood to mean mainly a polypeptide whose amino acid sequence contains one or more substitutions, additions or deletions of at least one amino acid residue, relative to the amino acid sequence of the reference polypeptide, it being understood that the amino acid substitutions may be either conservative or nonconservative.

“Fragment” of a polypeptide according to the invention will be understood to mean a polypeptide whose amino acid sequence is shorter than that of the reference polypeptide and which comprises, over the entire portion with these reference polypeptides, an identical amino acid sequence.

Such fragments may, where appropriate, be included in a larger polypeptide of which they are a part.

Such fragments of a polypeptide according to the invention may have a length of 10, 15, 20, 30 to 40, 50, 100, 200 or 300 amino acids.

The “percentage identity” between two nucleotide or amino acid sequences, for the purposes of the present invention, may be determined by comparing two sequences aligned optimally, through a window for comparison.

The portion of the nucleotide or polypeptide sequence in the window for comparison may thus comprise additions or deletions (for example “gaps”) relative to the reference sequence (which does not comprise these additions or these deletions) so as to obtain an optimum alignment of the two sequences.

The percentage is calculated by determining the number of positions at which an identical nucleic base or an identical amino acid residue is observed for the two sequences (nucleic or peptide) compared, and then by dividing the number of positions at which there is identity between the two bases or amino acid residues by the total number of positions in the window for comparison, and then multiplying the result by 100 in order to obtain the percentage sequence identity.

The optimum sequence alignment for the comparison may be achieved using a computer with the aid of known algorithms contained in the package from the company WISCONSIN GENETICS SOFTWARE PACKAGE, GENETICS COMPUTER GROUP (GCG), 575 Science Doctor, Madison, Wis.

By way of illustration, it will be possible to produce the percentage sequence identity with the aid of the BLAST software (versions BLAST 1.4.9 of March 1996, BLAST 2.0.4 of February 1998 and BLAST 2.0.6 of September 1998), using exclusively the default parameters (S. F Altschul et al, J. Mol. Biol. 1990 215 403-410, S. F Altschul et al, Nucleic Acids Res. 1997 25: 3389-3402). Blast searches for sequences similar/homologous to a reference “request” sequence, with the aid of the Altschul et al. algorithm. The request sequence and the databases used may be of the peptide or nucleic types, any combination being possible.

“High stringency hybridization conditions” for the purposes of the present invention will be understood to mean the following conditions:

1—Membrane Competition and Prehybridization:

Mix: 40 μl salmon sperm DNA (10 mg/ml)+40 μl human placental DNA (10 mg/ml)

Denature for 5 min at 96° C., then immerse the mixture in ice.

Remove the 2×SSC and pour 4 ml of formamide mix in the hybridization tube containing the membranes.

Add the mixture of the two denatured DNAs.

Incubation at 42° C. for 5 to 6 hours, with rotation.

2—Labeled Probe Competition:

Add to the labeled and purified probe 10 to 50 μl Cot I DNA, depending on the quantity of repeats.

Denature for 7 to 10 min at 95° C.

Incubate at 65° C. for 2 to 5 hours.

3—Hybridization:

Remove the prehybridization mix.

Mix 40 μl salmon sperm DNA+40 μl human placental DNA; denature for 5 min at 96° C., then immerse in ice.

Add to the hybridization tube 4 ml of formamide mix, the mixture of the two DNAs and the denatured labeled probe/Cot I DNA.

Incubate 15 to 20 hours at 42° C., with rotation.

4—Washes:

One wash at room temperature in 2×SSC, to rinse.

Twice 5 minutes at room temperature 2×SSC and 0.1% SDS at 65° C.

Twice 15 minutes at 65° C. 1×SSC and 0.1% SDS at 65° C.

Envelope the Membranes in Saran and Expose.

The hybridization conditions described above are adapted to hybridization, under high stringency conditions, of a molecule of nucleic acid of varying length from 20 nucleotides to several hundreds of nucleotides.

It goes without saying that the hybridization conditions described above may be adjusted as a function of the length of the nucleic acid whose hybridization is sought or of the type of labeling chosen, according to techniques known to persons skilled in the art.

Suitable hybridization conditions may for example be adjusted according to the teaching contained in the manual by HAMES and HIGGINS (1985) or in the manual by F. AUSUBEL et al (1999).

Nucleic Acids of the ABC1 Gene Genomic Sequences

The human ABC1 gene is thought to comprise 48 exons and 47 introns, if reference is made in particular to the structure of the orthologous ABC1 gene in mice.

Several partial genomic nucleotide sequences of the ABC1 gene have been isolated and characterized according to the invention, these genomic sequences comprising both new exonic sequences and intronic sequences which may be used in particular for the production of various means of detection of the ABC1 gene or of its nucleotide expression products in a sample. These partial genomic sequences are represented in Table 1 below.

TABLE I
Partial genomic sequences of the human ABC1 gene

| SEQ ID NO | Designation |
|---|---|
| 1 | Intron 10(p), exon 11(p) |
| 2 | Intron 11(p), exon 12, intron 12, exon |
| | 13, intron 13, exon 14, intron 14, exon |
| | 15, intron 15, exon 16, intron 16, exon |
| | 17(p) |
| 3 | Exon 17(p), intron 17(p) |
| 4 | Intron 18(p), exon 19, intron 19(p) |
| 5 | Intron 19(p), exon 20, intron 20, exon |
| | 21, intron 21, exon 22, intron 22, exon |
| | 23, intron 23, exon 24, intron 24, exon |
| | 25, intron 25, exon 26, intron 26(p) |
| 6 | Intron 26(p), exon 27, intron 27, exon |
| | 28, intron 28, exon 29, intron 29, exon |
| | 30, intron 30(p) |
| 7 | Intron 30(p), exon 31, intron 31, exon |
| | 32, intron 32, exon 33, intron 33, exon |
| | 34, intron 34(p) |
| 8 | Intron 34(p), exon 35, intron 35(p) |
| 9 | Intron 35(p), exon 36, intron 36(p) |
| 10 | Intron 36(p), exon 37, intron 37, exon |
| | 38, intron 38(p) |
| 11 | Intron 39(p), exon 40, intron 40, exon |
| | 41, intron 41(p) |
| 12 | Intron 41(p), exon 42, intron 42(p) |
| 13 | Intron 46(p), exon 47, intron 47(p) |
| 14 | Last exon(p), sequence in 3′ of the last |
| | exon |

Thus, a first subject of the invention consists in a nucleic acid comprising at least 245 consecutive nucleotides of a polynucleotide chosen from the group consisting of the nucleotide sequences SEQ ID NO 1-14, or a nucleic acid having a complementary sequence.

The invention also relates to a nucleic acid having at least 80%, advantageously 90%, preferably 95% and most preferably 98% nucleotide identity with a nucleic acid comprising at least 245 consecutive nucleotides of a polynucleotide chosen from the group consisting of the nucleotide sequences SEQ ID NO 1-14, or a nucleic acid having a complementary sequence.

Thirty two exons of the ABC1 gene have been characterized, at least partially, by their nucleotide sequence, as indicated in Table II below.

TABLE II

| | SEQ ID | Located in | Position of the | Position of the |
|---|---|---|---|---|
| Exon No. | NO | SEQ ID NO | nucleotide in 5′ | nucleotide in 3′ |
| 11 (5′ end) | 15 | 1 | 3003 | 3153 |
| 12 | 16 | 2 | 398 | 603 |
| 13 | 17 | 2 | 1124 | 1300 |
| 14 | 18 | 2 | 3087 | 3309 |
| 15 | 19 | 2 | 5055 | 5276 |
| 16 | 20 | 2 | 6337 | 6541 |
| 17 (5′ end) | 21 | 2 | 7646 | 7660 |
| 17 (3′ end) | 22 | 3 | 1 | 105 |
| 19 | 23 | 4 | 904 | 1035 |
| 20 | 24 | 5 | 284 | 426 |
| 21 | 25 | 5 | 630 | 767 |
| 22 | 26 | 5 | 1470 | 1690 |
| 23 | 27 | 5 | 2949 | 3021 |
| 24 | 28 | 5 | 4008 | 4210 |
| 25 | 29 | 5 | 5878 | 5926 |
| 26 | 30 | 5 | 6122 | 6235 |
| 27 | 31 | 6 | 561 | 709 |
| 28 | 32 | 6 | 2359 | 2483 |
| 29 | 33 | 6 | 3714 | 3812 |
| 30 | 34 | 6 | 6848 | 7036 |
| 31 | 35 | 7 | 1183 | 1277 |
| 32 | 36 | 7 | 2587 | 2619 |
| 33 | 37 | 7 | 3744 | 3849 |
| 34 | 38 | 7 | 5323 | 5397 |
| 35 | 39 | 8 | 236 | 405 |
| 36 | 40 | 9 | 989 | 1166 |
| 37 | 41 | 10 | 545 | 660 |
| 38 | 42 | 10 | 772 | 916 |
| 40 | 43 | 11 | 435 | 564 |
| 41 | 44 | 11 | 829 | 949 |
| 42 | 45 | 12 | 589 | 651 |
| 47 | 46 | 13 | 377 | 620 |
| Last exon | 47 | 14 | 1 | 1237 |
| (3′ end) | | | | |

Thus, the invention also relates to a nucleic acid comprising a polynucleotide chosen from the group consisting of the nucleotide sequences SEQ ID NO 15-47, or a nucleic acid having a complementary sequence.

Moreover, thirty-five introns of the ABC1 gene have been isolated and characterized, at least partially. The nucleotide sequences of the introns of the ABC1 gene, as well as their fragments and their variants may also be used as nucleotide probes or primers for detecting the presence of at least one copy of the ABC1 gene in a sample, or alternatively for amplifying a given target sequence in the ABC1 gene.

The references to the intronic sequences of the ABC1 gene are indicated in Table III below.

TABLE III

| | SEQ ID | Located in | Position of the | Position of the |
|---|---|---|---|---|
| Intron No. | NO | SEQ ID NO | nucleotide in 5′ | nucleotide in 3′ |
| 10 (3′ end) | 48 | 1 | 1 | 3002 |
| 11 (3′ end) | 49 | 2 | 1 | 397 |
| 12 | 50 | 2 | 604 | 1123 |
| 13 | 51 | 2 | 1301 | 3086 |
| 14 | 52 | 2 | 3310 | 5054 |
| 15 | 53 | 2 | 5277 | 6336 |
| 16 | 54 | 2 | 6542 | 7645 |
| 17 (3′ end) | 55 | 3 | 106 | 1285 |
| 18 (3′ end) | 56 | 4 | 1 | 903 |
| 19 (5′ end) | 57 | 4 | 1036 | 1521 |
| 19 (3′ end) | 58 | 5 | 1 | 283 |
| 20 | 59 | 5 | 427 | 629 |
| 21 | 60 | 5 | 768 | 1469 |
| 22 | 61 | 5 | 1691 | 2948 |
| 23 | 62 | 5 | 3022 | 4007 |
| 24 | 63 | 5 | 4211 | 5877 |
| 25 | 64 | 5 | 5927 | 6121 |
| 26 (5′ end) | 65 | 5 | 6236 | 6519 |
| 26 (3′ end) | 66 | 6 | 1 | 560 |
| 27 | 67 | 6 | 710 | 2358 |
| 28 | 68 | 6 | 2484 | 3713 |
| 29 | 69 | 6 | 3813 | 6847 |
| 30 (5′ end) | 70 | 6 | 7037 | 7378 |
| 30 (3′ end) | 71 | 7 | 1 | 1182 |
| 31 | 72 | 7 | 1278 | 2586 |
| 32 | 73 | 7 | 2620 | 3743 |
| 33 | 74 | 7 | 3850 | 5322 |
| 34 (5′ end) | 75 | 7 | 5398 | 5689 |
| 34 (3′ end) | 76 | 8 | 1 | 235 |
| 35 (5′ end) | 77 | 8 | 406 | 645 |
| 35 (3′ end) | 78 | 9 | 1 | 988 |
| 36 (5′ end) | 79 | 9 | 1167 | 1664 |
| 36 (3′ end) | 80 | 10 | 1 | 544 |
| 37 | 81 | 10 | 661 | 771 |
| 38 (5′ end) | 82 | 10 | 917 | 1279 |
| 39 (3′ end) | 83 | 11 | 1 | 434 |
| 40 | 84 | 11 | 565 | 828 |
| 41 (5′ end) | 85 | 11 | 950 | 1124 |
| 41 (3′ end) | 86 | 12 | 1 | 588 |
| 42 (5′ end) | 87 | 12 | 652 | 729 |
| 46 (3′ end) | 88 | 13 | 1 | 376 |
| 47 (5′ end) | 89 | 13 | 621 | 731 |
| Distal | 90 | 14 | 1238 | 3501 |
| sequence in | | | | |
| 3′ of the last | | | | |
| exon | | | | |

The invention also relates to a nucleic acid comprising at least 8 consecutive nucleotides of a polynucleotide chosen from the group consisting of the nucleotide sequences SEQ ID NO 48-89, or a nucleic acid having a complementary sequence.

The subject of the invention is, in addition, a nucleic acid having at least 80% nucleotide identity with a polynucleotide chosen from the group consisting of the nucleotide sequences SEQ ID NO 48-89, or a nucleic acid having a complementary sequence.

The invention also relates to a nucleic acid hybridizing, under high stringency conditions, with a polynucleotide chosen from the group consisting of the nucleotide sequences SEQ ID NO 48-89, or a nucleic acid having a complementary sequence.

In addition, a potentially regulatory genomic nucleotide sequence located downstream of the 3′ end of the last exon of the ABC1 gene has been isolated. It is a polynucleotide having the sequence SEQ ID NO 90. The characterization of polymorphisms in this potentially regulatory sequence (possible presence of regulatory sequences of the “enhancer” type), in particular in patients suffering from mild forms of deficiency in the reverse transport of cholesterol, in particular mild forms of Tangier disease, would be of the type allowing the production of appropriate means of detection, probes or primers, specific for some of these polymorphisms capable of inducing defects in the regulation of the expression of the ABC1 gene.

In order to identify the biologically active polynucleotide fragments of the sequence SEQ ID NO 90, persons skilled in the art can advantageously refer to the book by Sambrook et al. (1989) which describes the use of a recombinant vector carrying a marker gene (for example β-galactosidase, chloramphenicol acetyl transferase and the like) whose expression may be detected when this marker gene is placed under the control of a suitable promoter and of a biologically active fragment of the polynucleotide having the sequence SEQ ID NO 90. Such biologically active fragments of the sequence SEQ ID NO 90 may be in particular cloned into appropriate selection vectors having regulatory sequences, such as one of the vectors pSEAP-Basic, pSEAP-Enhancer, pβgal-Basic, pβgal-Enhancer, or pEGFP-1, marketed by the company Clontech.

The subject of the invention is, in addition, a nucleic acid having at least 80% nucleotide identity with a polynucleotide chosen from the group consisting of the nucleotide sequences SEQ ID NO 90, or a nucleic acid having a complementary sequence.

The invention also relates to a nucleic acid hybridizing, under high stringency conditions, with a polynucleotide chosen from the group consisting of the nucleotide sequences SEQ ID NO 90, or a nucleic acid having a complementary sequence.

Complete cDNA

As already indicated above, a partial cDNA sequence corresponding to the expression of the ABC1 gene has been identified by Langman et al. (1999). This partial cDNA sequence of ABC1 comprises 6880 nucleotides and contains the entire open reading frame corresponding to the ABC1 protein produced in subjects not affected by disorders linked to the reverse transport of cholesterol. The cDNA sequence described by Langmann et al. (1999) contains, in addition, a portion of the 5′-UTR region (nucleotides 1 to 120) and a portion of the 3′-UTR region (nucleotides 6727 to 6880).

The entire complete cDNA corresponding to the ABC1 gone, which corresponds to a new 3′-UTR region, which constitutes a major nucleic region, in particular from the point of view of the stability of the messenger RNAs in the cell, has now been isolated and characterized according to the invention.

The analyses of expression of the transcript having the sequence SEQ ID NO 91 were carried out by RT-PCR, as described in Example 1. These analyses carried out starting with the polyA+ RNA of various tissues have made it possible to ensure that the ABC1 gene was expressed in the fetal brain, the brain, the heart, the placenta and the uterus.

Consequently, the invention also relates to a nucleic acid comprising a polynucleotide having the sequence SEQ ID NO 91 of the cDNA of the human ABC1 gene, or a nucleic acid having a complementary sequence.

The cDNA of the human ABC1 gene having the sequence SEQ ID NO 91 comprises an open reading frame going from the nucleotide at position 121 (base A of the ATG codon for initiation of translation) to the nucleotide at position 6723 of the sequence SEQ ID NO 91. A polyadenylation signal (having the sequence ATTAAA) is present, starting at the nucleotide at position 9454 of the sequence SEQ ID NO 91.

The cDNA having the sequence SEQ ID NO 91 encodes the ABC1 polypeptide of 2201 amino acids in length, and having the amino acid sequence SEQ ID NO 139.

The invention also relates to a nucleic acid comprising at least eight consecutive nucleotides of a polynucleotide having the sequence SEQ ID NO 92, a biologically active fragment thereof or a nucleic acid having a complementary sequence.

The subject of the invention is also a nucleic acid having at least 80% nucleotide identity with a polynucleotide having the sequence SEQ ID NO 92, a biologically active fragment thereof or a nucleic acid having a complementary sequence.

Another subject of the invention consists in a nucleic acid hybridizing, under high stringency conditions, with a polynucleotide having the sequence SEQ ID NO 92, a biologically active fragment thereof or a nucleic acid having a complementary sequence.

Polymorphisms within the ABC1 Gene Mutations

According to the invention, several mutations have been identified in the sequence of the ABC1 gene, these mutations leading to major structural impairments of the ABC1 polypeptide encoded by the mutated sequences. These mutations have been found particularly in patients suffering from severe forms of Tangier disease, associated with serious coronary disorders. Two particularly deleterious mutations are described below.

1. Mutation in Exon 12

This mutation consists both in a deletion of a localized segment of 14 nucleotides (“TGAGAGGAAGTTCT”) from the nucleotide at position 472 to the nucleotide at position 485 of the normal genomic DNA having the sequence SEQ ID NO 2 and in an insertion of an Alu-type sequence of 110 nucleotides into the sequence of exon 12 of the ABC1 gene, upstream of the nucleotide at position 486 of the normal genomic DNA having the sequence SEQ ID NO 2.

The exon 12 carrying this deletion/insertion mutation has the nucleotide sequence SEQ ID NO 93.

The corresponding mutated cDNA has the nucleotide sequence SEQ ID NO 94, encodes a mutated ABC1 polypeptide of 2233 amino acids in length, having the sequence SEQ ID NO 140, whose structure is substantially altered compared with the normal ABC1 polypeptide having the sequence SEQ ID NO 139.

The nucleotide sequences SEQ ID NO 93 and 94 as well as the polypeptide sequence SEQ ID NO 140 also form part of the invention.

2 Mutation in Exon 13

This mutation consists of a deletion of the nucleotide (G) at position 1232 of the genomic sequence SEQ ID NO 2, which is located in exon 13 (nucleotide G at position 106 of the sequence of exon 13 SEQ ID NO 17). This point deletion of one base introduces a stop codon in the normal reading frame in the mRNA of the ABC 1 gene.

The sequence of exon 13 of the ABC1 gene carrying this mutation is the polynucleotide having the sequence SEQ ID NO 95.

The cDNA corresponding to this mutation in exon 13 of the ABC1 gene is represented in the nucleotide sequence SEQ ID NO 96.

The mutated protein encoded by the mutated ABC1 gene having a length of 574 amino acids, that is to say about a quarter of the length in terms of amino acids of the normal protein. The truncated polypeptide has the amino acid sequence SEQ ID NO 141.

The structural characteristics which make it possible to differentiate the normal sequences from the mutated sequences of ABC1 (genomic sequences, messenger RNAs, cDNA) may be exploited in order to produce means of detection of the mutated sequences of ABC1 in a sample, in particular probes specifically hybridizing with the mutated sequences of ABC1 or pairs of primers making it possible to selectively amplify the regions of the ABC1 gene carrying the mutations described above, it being possible to carry out the detection of the presence of these mutations in particular by distinguishing the length of the amplified nucleic acid fragments, by hybridization of the amplified fragments with the aid of the specific probes described above, or by direct sequencing of these amplified fragments.

Thus, a further subject of the invention is a nucleic acid having at least eight consecutive nucleotides of a polynucleotide chosen from the group consisting of the nucleotide sequences SEQ ID NO 93-96, or a nucleic acid having a complementary sequence.

Preferably, such a nucleic acid comprises:

a) either at least two consecutive nucleotides of the Alu sequence located in the sequences SEQ ID NO 93 and 94, preferably 5, 10, 15, 20, 25, 30, 35, 40, 50 or 100 consecutive nucleotides of the Alu sequence located in the sequences SEQ ID NO 93 and 94;

b) or at least the two “CT” nucleotides situated on either side of the deleted G base, in the sequences SEQ ID NO 94 and 95.

The primers hybridizing with a nucleic sequence located in the region of an ABC1 sequence (genomic sequence, messenger RNA) carrying either of the two mutations described above also form part of the invention.

The invention relates, in addition, to a nucleic acid having at least 80% nucleotide identity with a polynucleotide chosen from the group consisting of the nucleotide sequences SEQ ID NO 93-96, or a nucleic acid having a complementary sequence.

The invention also relates to a nucleic acid hybridizing, under high stringency conditions, with a polynucleotide chosen from the group consisting of the nucleotide sequences SEQ ID NO 93-96, or a nucleic acid having a complementary sequence.

Other Polymorphisms

Other polymorphisms have been found within the sequence of the ABC1 gene, in particular nucleotide substitutions located both in the coding regions (exons) and in the noncoding regions.

As regards the polymorphisms found in the coding regions, they are essentially substitutions of a single nucleotide located on the third base of the codons of the open reading frame of ABC1, these substitutions causing no modification as regards the nature of the amino acid encoded, taking into account the rules of genetic degeneration in humans, which are well known to persons skilled in the art.

These polymorphisms are represented in the present description in the form of nucleotide sequences of 41 bases in length, the polymorphic base being located at the center of the polymorphic fragment. For each of the polymorphisms identified, each allele is thus represented as a sequence of 41 bases, the polymorphism itself being defined by the two nucleotide sequences corresponding respectively to each of the forms. The polymorphisms identified in the ABC1 gene are represented in Table IV below.

TABLE IV
Polymorphisms found in the ABC1 gene

| | Position in the | | Allele 2 | Polymorphic |
|---|---|---|---|---|
| Designation | sequence | Allele 1 | SEQ | base |
| No. | SEQ ID NO 2 | SEQ ID NO | ID NO | Allele1/Allele 2 |
| 1 | 397 | 97 | 98 | G/A |
| 2 | 1324 | 99 | 100 | T/A |
| 3 | 3028 | 101 | 102 | C/T |
| 4 | 3234 | 103 | 104 | C/A |
| 5 | 3390 | 105 | 106 | A/G |
| 6 | 6854 | 107 | 108 | G/A |

The detection of these polymorphisms within a DNA sample obtained from a subject may, for example, be carried out by a specific amplification of the nucleotide region of ABC1 containing the polymorphic base, and then sequencing the amplified fragment in order to determine the nature of the allele or of the alleles carried by said subject.

The detection of these polymorphisms in a DNA sample obtained from a subject may also be carried out with the aid of nucleotide probes or primers specifically hybridizing with a given allele containing one of the polymorphic bases of a polymorphism of the ABC1 gene according to the invention.

By way of illustration, appropriate nucleotide primers are for example primers whose base at the 3′ end hybridizes with the base located immediately on the 5′ side of the polymorphic base of the fragment comprising said polymorphism. After the step of hybridization of the specific primer, a step of extension with a mixture of the two dideoxynucleotides complementary to the polymorphic base of said polymorphism, for example differentially labeled by fluorescence, and then a step detection of the fluorescence signal obtained makes it possible to determine which of the two differentially labeled fluorescent dideoxynucleotides has been incorporated and to directly deduce the nature of the polymorphic base present at the level of this polymorphism.

Various approaches may be used for the labeling and detection of the dideoxynucleotides. A method in homogeneous phase based on FRET (“Fluorescence resonance energy transfer”) has been described by Chen and Kwok (1997). According to this method, the amplified fragments of genomic DNA containing polymorphisms are incubated with a primer labeled with fluorescein at the 5′ end in the presence of labeled dideoxynucleotide triphosphate and a modified Taq polymerase. The labeled primer is extended by one base by incorporation of the labeled dideoxynucleotide specific for the allele present on the complementary genomic DNA sequence. At the end of this genotyping reaction, the fluorescence intensities for the two labeling compounds for the labeled dideoxynucleotides are directly analyzed without separation or purification. All these steps may be carried out in the same tube and the modifications of the fluorescence signal monitored in real time. According to another embodiment, the extended primer may be analyzed by MALDI-TOF type mass spectrometry. The base located at the level of the polymorphic site is identified by measuring the mass added to the microsequencing primer (Haff and Smirnov, 1997).

Such nucleotide primers may, for example, be demobilized on a support. Furthermore, it is possible to immobilize on a support, for example in an orderly manner, multiple specific primers as described above, each of the primers being suited to the detection of one of the polymorphisms of the ABC1 gene according to the invention.

The polymorphisms of the ABC1 gene according to the invention are useful in particular as genetic markers in studies of association between the presence of a given allele in a subject and the predisposition of this subject to a given pathology, in particular to one of the pathologies already associated with the chromosomal region 9q31 preferably with a pathology linked to a dysfunction in the reverse transport of cholesterol.

The methods for the genetic analysis of complex characters (phenotypes) are of various types (Lander and Schork, 1994). In general, the bialleleic polymorphisms according to the invention are useful in any of the methods described in the state of the art intended to demonstrate a statistically significant correlation between a genotype and a phenotype. The bialleleic polymorphisms may be used in linkage analyses and in allele sharing methods. Preferably, the bialleleic polymorphisms according to the invention are used to identify genes associated with detectable characters (phenotypes) in use for studies of association, an approach which does not require the use of families affected by the character, and which allows, in addition, the identification of genes associated with complex and sporadic characters.

Other statistical methods using bialleleic polymorphisms according to the invention are for example those described by Forsell et al. (1997), Xiong et al. (1999), Horvath et al. (1998), Sham et al. (1995) or Nickerson et al. (1992).

According to another aspect, the invention also relates to the nucleotide sequences of the ABC1 gene comprising at least one bialleleic polymorphism as described above.

Thus, the invention also relates to a nucleic acid having at least eight consecutive nucleotides of a polynucleotide chosen from the group consisting of the nucleotide sequences SEQ ID NO 97-108 and comprising the polymorphic base, or a nucleic acid having a complementary sequence.

Nucleotide Probes and Primers

The nucleic acid fragments derived from any one of the nucleotide sequences SEQ ID NO 1-14, 15-47, 48-89, 90, 92, 94-96 and 97-108 are useful for the detection of the presence of at least one copy of a nucleotide sequence of the ABC1 gene or of a fragment or of a variant (containing a mutation or a polymorphism) thereof in a sample.

The nucleotide probes or primers according to the invention comprise at least 8 consecutive nucleotides of a nucleic acid chosen from the group consisting of the sequences SEQ ID NO 1-14, 15-47, 48-89, 90, 92, 93-96 and 97-108, or of a nucleic acid having a complementary sequence.

Preferably, nucleotide probes or primers according to the invention will have a length of 10, 12, 15, 18 or 20 to 25, 35, 40, 50, 70, 80, 100, 200, 500, 1000, 1500 consecutive nucleotides of a nucleic acid according to the invention, in particular a nucleic acid having a nucleotide sequence chosen from the sequences SEQ ID NO 1-14, 15-47, 48-89, 90, 92, 93-96 and 97-108, or of a nucleic acid having a complementary sequence.

Alternatively, a nucleotide probe or primer according to the invention will consist of and/or comprise the fragments having a length of 12, 15, 18, 20, 25, 35, 40, 50, 100, 200, 500, 1000, 1500 consecutive nucleotides of a nucleic acid according to the invention, more particularly of a nucleic acid chosen from the sequences SE ID NO 1-14, 15-47, 48-89, 90, 92, 93-96 and 97-108, or of a nucleic acid having a complementary sequence.

The definition of a nucleotide probe or primer according to the invention therefore covers oligonucleotides which hybridize, under the high stringency hybridization conditions defined above, with a nucleic acid chosen from the sequences SEQ ID NO 1-14, 15-47, 48-89, 90, 92, 93-96 and 97-108 or with a sequence complementary thereto.

Examples of primers and pairs of primers which make it possible to amplify various regions of the ABC1 gene are presented In Table V below.

TABLE V
Primers for the amplification of nucleic fragments of the ABC1 gene

| | | | Sequence | |
|---|---|---|---|---|
| | Located in | Position in the | of the | Region for |
| Primer No. | SEQ ID | sequence | primer | hybridization |
| 1 | 2 | 313-335 | 109 | Intron 11 |
| 2 | 2 | Comp 640-663 | 110 | Intron 12 |
| 3 | 2 | 1005-1029 | 111 | Intron 12 |
| 4 | 2 | Comp 1472-1496 | 112 | Intron 13 |
| 5 | 2 | 2930-2954 | 113 | Intron 13 |
| 6 | 2 | Comp 3444-3468 | 114 | Intron 14 |
| 7 | 2 | 4988-5012 | 115 | Intron 14 |
| 8 | 2 | Comp 5338-5362 | 116 | Intron 15 |
| 9 | 2 | 6240-6262 | 117 | Intron 15 |
| 10 | 2 | Comp 6581-6603 | 118 | Intron 16 |
| 11 | 5 | 1369-1391 | 119 | Intron 21 |
| 12 | 5 | Comp 1748-1770 | 120 | Intron 22 |
| 13 | 5 | 3868-3890 | 121 | Intron 23 |
| 14 | 5 | Comp 4240-4262 | 122 | Intron 24 |
| 15 | 6 | 3587-3610 | 123 | Intron 28 |
| 16 | 6 | Comp 3881-3903 | 124 | Intron 29 |
| 17 | 6 | 6753-6775 | 125 | Intron 29 |
| 18 | 6 | Comp 7112-7134 | 126 | Intron 30 |
| 19 | 7 | 1060-1082 | 127 | Intron 30 |
| 20 | 7 | Comp 1377-1399 | 128 | Intron 31 |
| 21 | 7 | 3574-3596 | 129 | Intron 32 |
| 22 | 7 | Comp 3909-3931 | 130 | Intron 33 |
| 23 | 7 | 5161-5183 | 131 | Intron 33 |
| 24 | 7 | Comp 5463-5485 | 132 | Intron 34 |
| 25 | 8 | 100-122 | 133 | Intron 34 |
| 26 | 8 | Comp 475-497 | 134 | Intron 35 |
| 27 | 9 | 841-861 | 135 | Intron 35 |
| 28 | 9 | Comp 1249-1271 | 136 | Intron 36 |
| 29 | 10 | 455-477 | 137 | Intron 36 |
| 30 | 10 | Comp 966-988 | 138 | Intron 38 |

According to a first embodiment of preferred probes and primers according to the invention, they comprise all or part of a polynucleotide chosen from the nucleotide sequences SEQ ID NO 109-138, or nucleic acids having a complementary sequence.

A nucleotide primer or probe according to the invention may be prepared by any suitable method well known to persons skilled in the art, including by cloning and action of restriction enzymes or by direct chemical synthesis to techniques such as the phosphodiester method by Narang et al. (1979) or by Brown et al. (1979), the diethylphosphoramidite method by Beaucage et al. (1980) or the technique on a solid support described in EU patent. EP 0,707,592.

Each of the nucleic acids according to the invention, including the oligonucleotide probes and primers described above, may be labeled, if desired, by incorporating a marker which can be detected by spectroscopic, photochemical, biochemical, immunochemical or chemical means.

For example, such markers may consist of radioactive isotopes (32P, 33P, 3H, 35S), fluorescent molecules (5-bromodeoxyuridine, fluorescein, acetylaminofluorene, digoxigenin) or ligands such as biotin.

The labeling of the probes is preferably carried out by incorporating labeled molecules into the polynucleotides by primer extension, or alternatively by addition to the 5′ or 3′ ends.

Examples of nonradioactive labeling of nucleic acid fragments are described in particular in French patent No.78 109 75 or in the articles by Urdea et al. (1988) or Sanchez-pescador et al. (1988).

Advantageously, the probes according to the invention may have structural characteristics of the type to allow amplification of the signal, such as the probes described by Urdea et al. (1991.) or alternatively in European patent No. EP-0,225,807 (CHIRON).

The oligonucleotide probes according to the invention may be used in particular in Southern-type hybridizations with the genomic DNA or alternatively in hybridizations with the corresponding messenger RNA when the expression of the corresponding transcript is sought in a sample.

The probes according to the invention may also be used for the detection of products of PCR amplification or alternatively for the detection of mismatches.

Nucleotide probes or primers according to the invention may be immobilized on a solid support. Such solid supports are well known to persons skilled in the art and comprise surfaces of wells of microtiter plates, polystyrene beds, magnetic beds, nitrocellullose bands or microparticles such as latex particles.

Consequently, the present invention also relates to a method of detecting the presence of a nucleic acid as described above in a sample, said method comprising the steps of:

1) bringing one or more nucleotide probes according to the invention into contact with the sample to be tested;

2) detecting the complex which may have formed between the probe(s) and the nucleic acid present in the sample.

According to a specific embodiment of the method of detection according to the invention, the oligonucleotide probes are immobilized on a support.

According to another aspect, the oligonucleotide probes comprise a detectable marker.

The invention relates, in addition, to a box or kit for detecting the presence of a nucleic acid according to the invention in a sample, said box comprising:

a) one or more nucleotide probes as described above;

b) where appropriate, the reagents necessary for the hybridization reaction.

According to a first aspect, the detection box or kit is characterized in that the probe(s) are immobilized on a support.

According to a second aspect, the detection box or kit is characterized in that the oligonucleotide probes comprise a detectable marker.

According to a specific embodiment of the detection kit described above, such a kit will comprise a plurality of oligonucleotide probes in accordance with the invention which may be used to detect target sequences of interest or alternatively to detect mutations in the coding regions or the noncoding regions of the nucleic acids according to the invention, more particularly of the nucleic acids having the sequences SEQ ID NO 1-14, 15-47, 48-89, 90, 92, 93-96 and 97-108 or the nucleic acids having a complementary sequence.

Thus, the probes according to the invention, immobilized on a support, may be ordered into matrices such as “DNA chips”. Such ordered matrices have in particular been described in U.S. Pat. No. 5,143,854, in PCT applications No. WO 90/150 70 and 92/10092.

Support matrices on which oligonucleotide probes have been immobilized at a high density are for example described in U.S. Pat. No. 5,412,087 and in PCT application No. WO 95/11995.

The nucleotide primers according to the invention may be used to amplify any one of the nucleic acids according to the invention, and more particularly all or part of a nucleic acid having the sequences SEQ ID NO 1-14, 15-47, 48-89, 90, 92, 93-96 and 97-108, or alternatively a variant thereof.

Another subject of the invention relates to a method of amplifying a nucleic acid according to the invention, and more particularly a nucleic acid having the sequences SEQ ID NO 1-14, 15-47, 48-89, 90, 92, 93-96 and 97-108 or a fragment or a variant thereof contained in a sample, said method comprising the steps of:

a) bringing the sample in which the presence of the target nucleic acid is suspected into contact with a pair of nucleotide primers whose hybridization position is located respectively on the 5′ side and on the 3′ side of the region of the target nucleic acid whose amplification is sought, in the presence of the reagents necessary for the amplification reaction; and

b) detecting the amplified nucleic acids.

To carry out the amplification method as defined above, use will be advantageously made of any of the nucleotide primers described above.

The subject of the invention is, in addition, a box or kit for amplifying a nucleic acid according to the invention, and more particularly all or part of a nucleic acid having the sequences SEQ ID NO 1-14, 15-47, 48-89, 90, 92, 93-96 and 97-108, said box or kit comprising:

a) a pair of nucleotide primers in accordance with the invention, whose hybridization position is located respectively on the 5′ side and 3′ side of the target nucleic acid whose amplification is sought;

b) where appropriate, the reagents necessary for the amplification reaction.

Such an amplification box or kit will advantageously comprise at least one pair of nucleotide primers as described above.

According to a first preferred embodiment, primers according to the invention comprise all or part of a polynucleotide chosen from the nucleotide sequences SEQ ID NO 109 and 110, making it possible to amplify the region of exon 12 of the ABC1 gene carrying the first mutation (deletion/insertion) described above, or nucleic acids having a complementary sequence.

According to a second preferred embodiment, primers according to the invention comprise all or part of a polynucleotide chosen from the nucleotide sequences SEQ ID NO 111 and 112, making it possible to amplify the region of exon 13 of the ABC1 gene carrying the second mutation (deletion of a G base) described above, or nucleic acids having a complementary sequence.

According to a third preferred embodiment, primers according to the invention comprise, generally, all or part of a polynucleotide chosen from the nucleotide sequences SEQ ID NO 109-138, or nucleic acids having a complementary sequence.

According to a fourth preferred embodiment, the invention also relates to nucleotide primers comprising at least 15 consecutive nucleotides of a nucleic acid chosen from the group consisting of the sequences SEQ ID NO 97-108 or a nucleic acid having a complementary sequence, the base of the 3′ end of these primers being complementary to the nucleotide located immediately on the 5′ side of the polymorphic base of one of the sequences SEQ ID NO 97-108 or of their complementary sequences.

According to another aspect, the invention also relates to nucleotide primers comprising at least 15 consecutive nucleotides of a nucleic acid chosen from the group consisting of the sequences SEQ ID NO 97-108 or a nucleic acid having a complementary sequence, the base of the 3′ end of these primers being complementary to a nucleotide situated at 2, 3, 4, 5, 6, 7, 8, 9, 10, 11, 12, 13, 14, 15, 16, 17, 18, 19, 20 nucleotides or more on the 5′ side of the polymorphic base of one of the sequences SEQ ID NO 97-108 or of their complementary sequences. To construct primers whose nucleotide at the 3′ end is complementary to a nucleotide located at more than 20 nucleotides on the 5′ side of the polymorphic base of one of the sequences SEQ ID NO 97-108, persons skilled in the art will advantageously refer to the corresponding genomic sequence among the sequences SEQ ID NO 1-14 or SEQ ID NO 15-47 and 48-90, comprising the polymorphism for which the nature of the allele is sought.

Such primers are particularly useful in the context of methods of genotyping subjects and/or of genotyping populations, in particular in the context of studies of association between particular allele forms or particular forms of groups of alleles (haplotypes) in subjects and the existence of a particular phenotype (character) in these subjects, for example the predisposition of these subjects to develop diseases linked to a deficiency in the reverse transport of cholesterol, or alternatively the predisposition of these subjects to develop a pathology whose candidate chromosomal region is situated on chromosome 9, more precisely on the 9q arm and still more precisely in the 9q31 locus.

Recombinant Vectors

The invention also relates to a recombinant vector comprising a nucleic acid according to the invention.

Advantageously, such a recombinant vector will comprise a nucleic acid chosen from the following nucleic acids:

a) a nucleic acid having the sequence SEQ ID NO 92 or a biologically active fragment thereof;

b) a nucleic acid comprising a polynucleotide having the sequence SEQ ID NO 91, 94 or 96;

c) a nucleic acid comprising a polynucleotide chosen from the group consisting of the nucleotide sequences SEQ ID NO 15-47 and 48-90

d) a nucleic acid having at least 80% nucleotide identity with a nucleic acid chosen from the group consisting of the sequences SEQ ID N01547 and 48-90 or a fragment or a variant thereof;

d) a nucleic acid hybridizing, under high stringency hybridization conditions, with a nucleic acid having the sequences SEQ ID NO 15-47 and 48-90, or a fragment or a variant thereof.

“Vector” for the purposes of the present invention will be understood to mean a circular or linear DNA or RNA molecule which is either in single-stranded or double-stranded form.

According to a first embodiment, a recombinat vector according to the invention is used to amplify the nucleic acid which is inserted therein after transformation or transfection of the desired cellular host.

According to a second embodiment, it corrresponds to expression vectors comprising, in addition to a nucleic acid in accordance with the invention, regulatory sequences which make it possible to direct the transcription and/or translation thereof.

According to an advantageous embodiment, a recombinant vector according to the invention will comprise in particular the following elements:

(1) elements for regulating the expression of the nucleic acid to be inserted, such as promoters and enhancer sequences;

(2) the coding sequence contained in the nucleic acid in accordance with the invention to be inserted into such a vector, said coding sequence being placed in phase with the regulatory signals described in (1); and

(3) appropriate sequences for initiation and termination of the transcription.

In addition, the recombinant vectors according to the invention may include one or more origins for replication in the cellular hosts in which their amplification or their expression is sought, markers or selectable markers.

By way of example, the bacterial promoters may be the LacI or LacZ promoters, the T3 or T7 bacteriophage RNA polymerase promoters, the lambda phage PR or PL promoters.

The promoters for eukaryotic cells will comprise the HSV virus thymidine kinase promoter or alternatively the mouse metallothionein-L promoter.

Generally, for the choice of a suitable promoter, persons skilled in the art can advantageously refer to the book by Sambrook et al. (1989) cited above or to the techniques described by Fuller et al. (1996).

When the expression of the genomic sequence of the ABC1 gene will be sought, use will preferably be made of the vectors capable of containing large insertion sequences. In this particular embodiment, bacteriophage vectors such as the P1 bacteriophage vectors such as the vector p158 or the vector p158/neo8 described by Sternberg (1992, 1994) will be preferably used.

The preferred bacterial vectors according to the invention are for example the vectors pBR322(ATCC37017) or alternatively vectors such as pAA223-3 (Pharmacia, Uppsala, Sweden), and pGEM1 (Promega Biotech, Madison, Wis., UNITED STATES).

There may also be cited other commercially available vectors such as the vectors pQE70, pQE60, pQE9 (Qiagen), psiX174, pBluescript SA, pNH8A, pNH16A, pNH18A, pNH46A, pWLNEO, pSV2CAT, pOG44, pXTI, pSG(Stratagene).

They may also be vectors of the baculovirus type such as the vector pVL1392/1393 (Pharmingen) used to transfect cells of the Sf9 line (ATCC No. CRL 1711) derived from Spodoptera frugiperda.

They may also be adenoviral vectors such as the human adenovirus of type 2 or 5.

A recombinant vector according to the invention may also be a retroviral vector or an adeno-associated vector (AAV). Such adeno-associated vectors are for example described by Flotte et al. (1992), Samulski et al. (1989), or McLaughlin BA et al. (1996).

To allow the expression of the polynucleotides according to the invention, the latter must be introduced into a host cell. The introduction of the polynucleotides according to the invention into a host cell may be carried out in vitro, according to the techniques well known to persons skilled in the art for transforming or transfecting cells, either in primer culture, or in the form of cell lines. It is also possible to carry out the introduction of the polynucleotides according to the invention in vivo or ex vivo, for the prevention or treatment of diseases linked to a deficiency in the reverse transport of cholesterol.

To introduce the polynucleotides or the vectors into a host cell, persons skilled in the art can advantageously refer to various techniques, such as the calcium phosphate precipitation technique (Graham et al., 1973; Chen et al., 1987), DEAE Dextran (Gopal,. 1985), electroporation (Tur-Kaspa, 1896; Potter et al., 1984), direct microinjection (Harland et al., 1985), liposomes charged with DNA (Nicolau et al., 1982, Fraley et al., 1979).

Once the polynucleotide has been introduced into the host cell, it may be stably integrated into the genome of the cell. The intregration may be achieved at a precise site of the genome, by homologous recombination, or it may be randomly integrated in some embodiments, the polynucleotide may be stably maintained in the host cell in the form of an episome fragment, the episome comprising sequences allowing the retention and the replication of the latter, either independently, or in a synchronized manner with the cell cycle.

According to a specific embodiment, a method of introducing a polynucleotide according to the invention into a host cell, in particular a host cell obtained from a mammal, in-vivo, comprises a step during which a preparation comprising a pharmaceutically compatible vector and a “naked” polynucleotide according to the invention, placed under the control of appropriate regulatory sequences, is introduced by local injection at the level of the chosen tissue, for example a smooth muscle tissue, the “naked” polynucleotide being absorbed by the cells of this tissue.

Compositions for use in vitro and in vivo comprising “naked” polynucleotides are for example described in PCT Application No. WO 95/11307 (Institut Pasteur, Inserm, University of Ottawa) as well as in the articles by Tacson et al. (1996) and Huygen et al. (1996).

According to a specific embodiment of the invention, a composition is provided for the in vivo production of the ABC1 protein. This composition comprises a polynucleotide encoding the ABC1 polypeptide placed under the control of appropriate regulatory sequences, in solution in a physiologically acceptable vector.

The quantity of vector which is injected into the host organism chosen varies according to the site of the injection. As a guide, there may be injected between about 0.1 and about 100 μg of polynucleotide encoding the ABC1 protein years the body of an animal, preferably of a patient likely to develop a disease linked to a deficiency in the reverse transport of cholesterol or who has already developed this disease, in particular a patient having a predisposition to Tangier disease or who has already developed the disease.

Consequently, the invention also relates to a pharmaceutical composition intended for the prevention of or treatment of subjects affected by, a dysfunction in the reverse transport of cholesterol, comprising a nucleic acid encoding the ABC1 protein, in combination with one or more physiologically compatible excipients.

Advantageously, such a composition will comprise the polynucleotide having the sequence SEQ ID NO 91, placed under the control of appropriate regulatory elements.

The subject of the invention is, in addition, a pharmaceutical composition intended for the prevention of or treatment of subjects affected by, a dysfunction in the reverse transport of cholesterol, comprising a recombinant vector according to the invention, in combination with one or more physiologically compatible excipients.

The invention also relates to the use of a nucelic acid according to the invention, encoding the ABC1 protein, for the manufacture of a medicament intended for the prevention of Atherosclerosis in various forms or more particularly for the treatment of subjects affected by a dysfunction in the reverse transport of cholesterol.

The invention also relates to the use of a recombinant vector according to the invention, comprising a nucleic acid encoding the ABC1 protein, for the manufacture of a medicament intended for the prevention of Atherosclerosis in various forms or more particularly for the treatment of subjects affected by a dysfunction in the reverse transport of cholesterol.

Vectors Useful in Methods of Somatic Gene Therapy and Compositions Containing Such Vectors

The present invention also relates to a new therapeutic approach for the treatment of pathologies linked to the transport of cholesterol. It provides an advantageous solution to the disadvantages of the prior art, by demonstrating the possibility of treating the pathologies linked to the transport of cholesterol by gene therapy, by the transfer and expression in vivo of a gene encoding an ABC1 protein involved in the transport and the metabolism of cholesterol. The invention thus offers a simple means allowing a specific and effective treatment of related pathologies such as, for example, atherosclerosis.

Gene therapy consists in correcting a deficiency or an abnormality. (mutation, aberrant expression and the like) and in bringing about the expression of a protein of therapeutic interest by introducing genetic information into the affected cell or organ. This genetic information may be introduced either ex vivo into a cell extracted from the organ, the modified cell then being reintroduced into the body, or directly in vivo into the appropriate tissue. In this second case, various techniques exist, among which various transfection techniques involving complexes of DNA and DEAE-dextran (Pagano et al., J. Virol. 1 (1967) 891), of DNA and nuclear proteins (Kaneda et al., Science 243 (1989) 375), of DNA and lipids (Feigner et al., PNAS 84 (1987) 7413), the use of liposomes (Fraley et al., J. Biol. Chem. 255 (1980) 10431), and the like. More recently, the use of viruses as vectors for the transfer of genes has appeared as a promising alternative to these physical transfection techniques. In this regard, various viruses have been tested for their capacity to infect certain cell populations. In particular, the retroviruses (RSV, HMS, MMS, and the like), the HSV virus, the adeno-associated viruses and the adenoviruses.

The present invention therefore also relates to a new therapeutic approach for the treatment of pathologies linked to the transport of cholesterol, consisting in transferring and in expressing in vivo genes encoding ABC1. In a particularly advantageous manner, the applicant has now found that it is possible to construct recombinant viruses containing a DNA sequence encoding an ABC1 protein involved in the metabolism of cholesterol, to administer these recombinant viruses in vivo, and that this administration allows a stable and effective expression of a biologically active ABC1 protein in vivo, with no cytopathological effect.

The present invention also results from the demonstration that adenoviruses constitute particularly efficient vectors for the transfer and the expression of the ABC1 gene. In particular, the present invention shows that the use of recombinant adenoviruses as vectors makes it possible to obtain sufficiently high levels of expression of this gene to produce the desired therapeutic effect. Other viral vectors such as retroviruses or adeno-associated viruses (AAV) allowing a stable expression of the gene are also claimed.

The present invention thus offers a new approach for the treatment and prevention of cardiovascular and neurological pathologies linked to the abnormalities of the transport of cholesterol.

The subject of the invention is therefore also a defective recombinant virus comprising a nucleic sequence encoding an ABC1 protein involved in the metabolism of cholesterol.

The invention also relates to the use of such a defective recombinant virus for the preparation of a pharmaceutical composition intended for the treatment and/or for the prevention of cardiovascular diseases.

The present invention also relates to the use of cells genetically modified ex vivo with a virus as described above, or of producing cells such as viruses, implanted in the body, allowing a prolonged and effective expression in vivo of a biologically active ABC1 protein.

The present invention shows that it is possible to incorporate a DNA sequence encoding ABC1 into a viral vector, and that these vectors make it possible to effectively express a biologically active, mature form. More particularly, the invention shows that the in vivo expression of ABC1 may be obtained by direct administration of an adenovirus or by implantation of a producing cell or of a cell genetically modified by an adenovirus or by a retrovirus incorporating such a DNA.

The present invention is particularly advantageous because it makes it possible to induce a controlled expression, and with no harmful effect, of ABC1 in organs which are not normally involved in the expression of this protein. In particular, a significant release of the ABC1 protein is obtained by implantation of cells producing vectors of the invention, or infected ex vivo with vectors of the invention.

The activity of transport of cholesterol produced in the context of the present invention may be of the human or animal ABC1 type. The nucleic sequence used in the context of the present invention may be a cDNA, a genomic DNA (gDNA), an RNA (in the case of retroviruses) or a hybrid construct consisting, for example, of a cDNA into which one or more introns would be inserted. It may also involve synthetic or semisynthetic sequences. In a particularly advantageous manner, a cDNA or a gDNA is used. In particular, the use of a gDNA allows a better expression in human cells. To allow their incorporation into a viral vector according to the invention, these sequences are advantageously modified, for example by site-directed mutagenesis, in particular for the insertion of appropriate restriction sites. The sequences described in the prior art are indeed not constructed for use according to the invention, and prior adaptations may prove necessary, in order to obtain substantial expressions. In the context of the present invention, the use of a nucleic sequence encoding a human ABC1 protein is preferred. Moreover, it is also possible to use a construct encoding a derivative of these ABC1 proteins. A derivative of these ABC1 proteins comprises, for example, any sequence obtained by mutation, deletion and/or addition relative to the native sequence, and encoding a product retaining the cholesterol transport activity. These modifications may be made by techniques known to a person skilled in the art (see general molecular biological techniques below). The biological activity of the derivatives thus obtained can then be easily determined, as indicated in particular in the examples of the measurement of the efflux of cholesterol from cells. The derivatives for the purposes of the invention may also be obtained by hybridization from nucleic acid libraries, using as probe the native sequence or a fragment thereof.

These derivatives are in particular molecules having a higher affinity for their binding sites, molecules exhibiting greater resistance to proteases, molecules having a higher therapeutic efficacy or fewer side effects, or optionally new biological properties. The derivatives also include the modified DNA sequences allowing improved expression in vivo.

In a first embodiment, the present invention relates to a defective recombinant virus comprising a cDNA sequence encoding an ABC1 protein involved in the transport and metabolism of cholesterol In another preferred embodiment of the invention, the DNA sequence is a gDNA sequence.

The vectors of the invention may be prepared from various types of viruses. Preferably, vectors derived from adenoviruses, adeno-associated viruses (AAV), herpesviruses (HSV) or retroviruses are used. It is most particularly advantageous to use an adenovirus, for direct administration or for the ex vivo modification of cells intended to be implanted, or a retrovirus, for the implantation of producing cells.

The viruses according to the invention are defective, that is to say that they are incapable of autonomously replicating in the target cell. Generally, the genome of the defective viruses used in the context of the present invention therefore lacks at least the sequences necessary for the replication of said virus in the infected cell. These regions may be either eliminated (completely or partially), or made nonfunctional, or substituted with other sequences and in particular with the nucleic sequence encoding the ABC1 protein. Preferably, the defective virus retains, nevertheless, the sequences of its genome which are necessary for the encapsidation of the viral particles.

As regards more particularly adenoviruses, various serotypes, whose structure and properties vary somewhat, have been characterized. Among these serotypes, human adenoviruses of type 2 or 5 (Ad 2 or Ad 5) or adenoviruses of animal origin (see Application WO 94/26914) are preferably used in the context of the present invention. Among the adenoviruses of animal origin which can be used in the context of the present invention, there may be mentioned adenoviruses of canine, bovine, murine (example: Mav1, Beard et al., Virology 75 (1990) 81), ovine, porcine, avian or simian (example: SAV) origin. Preferably, the adenovirus of animal origin is a canine adenovirus, more preferably a CAV2 adenovirus [Manhattan or A26161 strain (ATCC VR-800) for example]. Preferably, adenoviruses of human or canine or mixed origin are used in the context of the invention. Preferably, the defective adenoviruses of the invention comprise the ITRs, a sequence allowing the encapsidation and the sequence encoding the ABC1 protein. Advantageously, in the genome of the adenoviruses of the invention, the E1 region at least is made nonfunctional. Still more preferably, in the genome of the adenoviruses of the invention, the E1 gene and at least one of the E2, E4 and L1-L5 genes are nonfunctional. The viral gene considered may be made nonfunctional by any technique known to a person skilled in the art, and in particular by total suppression, by substitution, by partial deletion or by addition of one or more bases in the gene(s) considered. Such modifications may be obtained in vitro (on the isolated DNA) or in situ, for example, by means of genetic engineering techniques, or by treatment by means of mutagenic agents. Other regions may also be modified, and in particular the E3 (WO95/02697), E2 (WO94/28938), E4 (WO94/28152, WO94/12649, WO95/02697) and L5 (WO95/02697) region. According to a preferred embodiment, the adenovirus according to the invention comprises a deletion in the E1 and E4 regions and the sequence encoding ABC1 is inserted at the level of the inactivated E1 region. According to another preferred embodiment, it comprises a deletion in the E1 region at the level of which the E4 region and the sequence encoding ABC1 (French Patent Application FR94 13355) are inserted.

The defective recombinant adenoviruses according to the invention may be prepared by any technique known to persons skilled in the art (Levrero et al., Gene 101 (1991) 195, EP 185 573; Graham, EMBO J. 3 (1984) 2917). In particular, they may be prepared by homologous recombination between an adenovirus and a plasmid carrying, inter alia, the DNA sequence encoding the ABC1 protein. The homologous recombination occurs after cotransfection of said adenoviruses and plasmid into an appropriate cell line. The cell line used must preferably (i) be transformable by said elements, and (ii), contain the sequences capable of complementing the part of the defective adenovirus genome, preferably in integrated form in order to avoid the risks of recombination. By way of example of a line, there may be mentioned the human embryonic kidney line 293 (Graham et al., J. Gen. Virol. 36 (1977) 59) which contains in particular, integrated into its genome, the left part of the genome of an Ad5 adenovirus (12%) or lines capable of complementing the E1 and E4 functions as described in particular in Applications No. WO 94/26914 and WO95/02697.

Next, the adenoviruses which have multiplied are recovered and purified according to conventional molecular biological techniques, as illustrated in the examples.

As regards the adeno-associated viruses (AAV), they are DNA viruses of a relatively small size, which integrate into the genome of the cells which they infect, in a stable and site-specific manner. They are capable of infecting a broad spectrum of cells, without inducing any effect on cellular growth, morphology or differentiation. Moreover, they do not appear to be involved in pathologies in humans. The genome of AAVs has been cloned, sequenced and characterized. It comprises about 4700 bases, and contains at each end an inverted repeat region (ITR) of about 145 bases, serving as replication origin for the virus. The remainder of the genome is divided into 2 essential regions carrying the encapsidation functions: the left hand part of the genome, which contains the rep gene, involved in the viral replication and the expression of the viral genes; the right hand part of the genome, which contains the cap gene encoding the virus capsid proteins.

The use of vectors derived from AAVs for the transfer of genes in vitro and in vivo has been described in the literature (see in particular WO 91/18088; WO 93/09239; U.S. Pat. No. 4,797,368, U.S. Pat. No. 5,139,941, EP 488 528). These applications describe various constructs derived from AAVs, in which the rep and/or cap genes are deleted and replaced by a gene of interest, and their use for transferring in vitro (on cells in culture) or in vivo (directly into an organism) said gene of interest. However, none of these documents either describes or suggests the use of a recombinant AAV for the transfer and expression in vivo or ex vivo of an ABC1 protein, or the advantages of such a transfer. The defective recombinant AAVs according to the invention may be prepared by cotransfection, into a cell line infected with a human helper virus (for example an adenovirus), of a plasmid containing the sequence encoding the ABC1 protein bordered by two AAV inverted repeat regions (ITR), and of a plasmid carrying the AAV encapsidation genes (rep and cap genes). The recombinant AAVs produced are then purified by conventional techniques.

As regards the herpesviruses and the retroviruses, the construction of recombinant vectors has been widely described in the literature: see in particular Breakfield et al., New Biologist 3 (1991) 203; EP 453242, EP178220, Bernstein et al. Genet. Eng. 7 (1985) 235; McCormick, BioTechnology 3 (1985) 689, and the like.

In particular, the retroviruses are integrating viruses, infecting dividing cells. The genome of the retroviruses essentially comprises two LTRs, an encapsidation sequence and three coding regions (gag, pol and env). In the recombinant vectors derived from retroviruses, the gag, pol and env genes are generally deleted, completely or partially, and replaced with a heterologous nucleic acid sequence of interest. These vectors may be produced from various types of retroviruses such as in particular MoMuLV (“murine moloney leukemia virus”; also called MoMLV), MSV (“murine moloney sarcoma virus”), HaSV (“harvey sarcoma virus”); SNV (“spleen necrosis virus”); RSV (“rous sarcoma virus”) or Friend's virus.

To construct recombinant retroviruses containing a sequence encoding the ABC1 protein according to the invention, a plasmid containing in particular the LTRs, the encapsidation sequence and said coding sequence is generally constructed, and then used to transfect a so-called encapsidation cell line, capable of providing in trans the retroviral functions deficient in the plasmid.

Generally, the encapsidation lines are therefore capable of expressing the gag, pol and env genes. Such encapsidation lines have been described in the prior art, and in particular the PA317 line (U.S. Pat. No. 4,861,719), the PsiCRIP line (WO 90/02806) and the GP+envAm-12 line (WO 89107150). Moreover, the recombinant retroviruses may contain modifications at the level of the LTRs in order to suppress the transcriptional activity, as well as extended encapsidation sequences, containing a portion of the gag gene (Bender et al., J. Virol. 61 (1987) 1639). The recombinant retroviruses produced are then purified by conventional techniques.

To carry out the present invention, it is most particularly advantageous to use a defective recombinant adenovirus. The results given below indeed demonstrate the particularly advantageous properties of adenoviruses for the in vivo expression of a protein having a cholesterol transport activity. The adenoviral vectors according to the invention are particularly advantageous for a direct administration in vivo of a purified suspension, or for the ex vivo transformation of cells, in particular autologous cells, in view of their implantation. Furthermore, the adenoviral vectors according to the invention exhibit, in addition, considerable advantages, such as in particular their very high infection efficiency, which makes it possible to carry out infections using small volumes of viral suspension.

According to another particularly advantageous embodiment of the invention, a line producing retroviral vectors containing the sequence encoding the ABC1 protein is used for implantation in vivo. The lines which can be used to this end are in particular the PA317 (U.S. Pat. No. 4,861,719), PsiCrip (WO 90/02806) and GP+envAm-12 (U.S. Pat. No. 5,278,056) cells modified so as to allow the production of a retrovirus containing a nucleic sequence encoding an ABC1 protein according to the invention. For example, totipotent stem cells, precursors of blood cell lines, may be collected and isolated from a subject. These cells, when cultured, may then be transfected with the retroviral vector containing the sequence encoding the ABC1 protein under the control of viral, nonviral or nonviral promoters specific for macrophages or under the control of its own promoter. These cells are then reintroduced into the subject. The differentiation of these cells will be responsible for blood cells expressing the ABC1 protein, in particular for monocytes which, when transformed to macrophages, participate in the removal of cholesterol from the arterial wall. These macrophages expressing the ABC1 protein will have an increased capacity to metabolize cholesterol in excess and will make it available to the cell surface for its removal by the primary acceptors of membrane cholesterol.

Advantageously, in the vectors of the invention, the sequence encoding the ABC1 protein is placed under the control of signals allowing its expression in the infected cells. These may be expression signals which are homologous or heterologous, that is to say signals different from those which are naturally responsible for the expression of the ABC1 protein. They may also be in particular sequences responsible for the expression of other proteins, or synthetic sequences. In particular, they may be sequences of eukaryotic or viral genes or derived sequences, stimulating or repressing the transcription of a gene in a specific manner or otherwise and in an inducible manner or otherwise. By way of example, they may be promoter sequences derived from the genome of the cell which it is desired to infect, or from the genome of a virus, and in particular the promoters of the E1A or MLP genes of adenoviruses, the CMV promoter, the RSV-LTR and the like. Among the eukaryotic promoters, there may also be mentioned the ubiquitous promoters (HPRT, vimentin, α-actin, tubulin and the like), the promoters of the intermediate filaments (desmin, neurofilaments, keratin, GFAP, and the like), the promoters of therapeutic genes (of the MDR, CFTR or factor VIII type, and the like), tissue-specific promoters (pyruvate kinase, villin, promoter of the fatty acid binding intestinal protein, promoter of the smooth muscle cell α-actin, promoters specific for the liver; Apo Al, Apo All, human albumin and the like) or promoters corresponding to a stimulus (steroid hormone receptor, retinoic acid receptor and the like). In addition, these expression sequences may be modified by addition of enhancer or regulatory sequences and the like. Moreover, when the inserted gene does not contain expression sequences, it may be inserted into the genome of the defective virus downstream of such a sequence.

In a specific embodiment, the invention relates to a defective recombinant virus comprising a nucleic sequence encoding an ABC1 protein involved in the metabolism of cholesterol under the control of a promoter chosen from RSV-LTR or the CMV early promoter.

As indicated above, the present invention also relates to any use of a virus as described above for the preparation of a pharmaceutical composition for the treatment and/or prevention of pathologies linked to the transport of cholesterol.

The present invention also relates to a pharmaceutical composition comprising one or more defective recombinant viruses as described above. These pharmaceutical compositions may be formulated for administration by the topical, oral, parenteral, intranasal, intravenous, intramuscular, subcutaneous, intraocular or transdermal route and the like. Preferably, the pharmaceutical compositions of the invention contain a pharmaceutically acceptable vehicle for an injectable formulation, in particular for an intravenous injection, such as for example into the patients portal vein. They may relate in particular to isotonic sterile solutions or dry, in particular, freeze-dried, compositions which, upon addition depending on the case of sterilized water or physiological saline, allow the preparation of injectable solutions. Direct injection into the patient's portal vein is advantageous because it makes it possible to target the infection at the level of the liver and thus to concentrate the therapeutic effect at the level of this organ.

The doses of defective recombinant virus used for the injection may be adjusted as a function of various parameters, and in particular as a function of the viral vector, of the mode of administration used, of the relevant pathology or of the desired duration of treatment. In general, the recombinant adenoviruses according to the invention are formulated and administered in the form of doses of between 10⁴ and 10¹⁴ pfu/ml, and preferably 10⁶ to 10¹⁰ pfu/ml. The term pfu (“plaque forming unit”) corresponds to the infectivity of a virus solution, and is determined by infecting an appropriate cell culture and measuring, generally after 48 hours, the number of plaques of infected cells. The techniques for determining the pfu titer of a viral solution are well documented in the literature.

As regards retroviruses, the compositions according to the invention may directly contain the producing cells, with a view to their implantation.

In this regard, another subject of the invention relates to any mammalian cell infected with one or more defective recombinant viruses as described above. More particularly, the invention relates to any population of human cells infected with these viruses. These may be in particular cells of blood origin (totipotent stem cells or precursors), fibroblasts, myoblasts, hepatocytes, keratinocytes, smooth muscle and endothelial cells, glial cells and the like.

The cells according to the invention may be derived from primary cultures. These may be collected by any technique known to persons skilled in the art and then cultured under conditions allowing their proliferation. As regards more particularly fibroblasts, these may be easily obtained from biopsies, for example according to the technique described by Ham [Methods Cell. Biol. 21a (1980) 255]. These cells may be used directly for infection with the viruses, or stored, for example by freezing, for the establishment of autologous libraries, in view of a subsequent use. The cells according to the invention may be secondary cultures, obtained for example from preestablished libraries (see for example EP 228458, EP 289034, EP 400047, EP 456640).

The cells in culture are then infected with the recombinant viruses, in order to confer on them the capacity to produce a biologically active ABC1 protein. The infection is carried out in vitro according to techniques known to persons skilled in the art. In particular, depending on the type of cells used and the desired number of copies of virus per cell, persons skilled in the art can adjust the multiplicity of infection and optionally the number of infectious cycles produced. It is clearly understood that these steps must be carried out under appropriate conditions of sterility when the cells are intended for administration in vivo. The doses of recombinant virus used for the infection of the cells may be adjusted by persons skilled in the art according to the desired aim. The conditions described above for the administration in vivo may be applied to the infection in vitro. For the infection with retroviruses, it is also possible to coculture the cells which it is desired to infect with cells producing the recombinant retroviruses according to the invention. This makes it possible to dispense with the purification of the retroviruses.

Another subject of the invention relates to an implant comprising mammalian cells infected with one or more defective recombinant viruses as described above or cells producing recombinant viruses, and an extracellular matrix. Preferably, the implants according to the invention comprise 10⁵ to 1⁰¹⁰ cells. More preferably, they comprise 10⁶ to 10⁸ cells.

More particularly, in the implants of the invention, the extracellular matrix comprises a gelling compound and optionally a support allowing the anchorage of the cells.

For the preparation of the implants according to the invention, various types of gelling agents may be used. The gelling agents are used for the inclusion of the cells in a matrix having the constitution of a gel, and for promoting the anchorage of the cells on the support, where appropriate. Various cell adhesion agents can therefore be used as gelling agents, such as in particular collagen, gelatin, glycosaminoglycans, fibronectin, lectins and the like. Preferably, collagen is used in the context of the present invention. This may be collagen of human, bovine or murine origin. More preferably, type I collagen is used.

As indicated above, the compositions according to the invention advantageously comprise a support allowing the anchorage of the cells. The term anchorage designates any form of biological and/or chemical and/or physical interaction causing the adhesion and/or the attachment of the cells to the support. Moreover, the cells may either cover the support used, or penetrate inside this support, or both. It is preferable to use in the context of the invention a solid, nontoxic and/or biocompatible support. In particular, it is possible to use polytetrafluoroethylene (PTFE) fibers or a support of biological origin.

The present invention thus offers a very effective means for the treatment or prevention of pathologies linked to the transport of cholesterol, in particular obesity, hypertriglyceridemia, or, in the field of cardiovascular conditions, myocardial infarction, angina, sudden death, cardiac decompensation and cerebrovascular accidents.

In addition, this treatment may be applied to both humans and any animals such as ovines, bovines, domestic animals (dogs, cats and the like), horses, fish and the like.

Recombinant Host Cells

The invention also relates to a recombinant host cell comprising any of the nucleic acids of the invention, and more particularly a nucleic acid having the sequence SEQ ID NO 91, 94 or 96

According to another aspect, the invention also relates to a recombinant host cell comprising a recombinant vector as described above.

The preferred host cells according to the invention are for example the following:

a) prokaryotic host cells: strains of Escherichia coli (strain DH5-α), of Bacillus subtilis, of Salmonella typhimurium, or strains of species such as Pseudomonas, Streptomyces and Staphylococus;

b) eukaryotic host cells: HeLa cells (ATCC No. CCL2), Cv 1 cells (ATCC No. CCL70), COS cells (ATCC No. CRL 1650), Sf-9 cells (ATCC No. CRL 1711), CHO cells (ATCC No. CCL-61) or 3T3 cells (ATCC No. CRL-6361).

Mutated ABC1 Polypeptides

According to another aspect, the invention relates to a polypeptide encoded by a mutated ABC1 gene, and more particularly a mutated ABC1 gene in patients suffering from a deficiency in the reverse transport of cholesterol, most particularly in patients suffering from Tangier disease.

As indicated above, two deleterious mutations have been identified in patients suffering from Tangier disease.

The first mutation corresponds to the insertion of a fragment of about one hundred base pairs into the coding sequence, at the level of exon 12 of the ABC1 gene, leading to the production of a biologically inactive polypeptide of 2233 amino acids having the sequence SEQ ID NO 140. The mutated ABC1 polypeptide having the sequence SEQ ID NO 140 possesses, compared with the normal polypeptide having the sequence SEQ ID NO 139, the following differences:

a) a deletion of a peptide fragment having the sequence “DERKFW” and the replacement of this peptide fragment with the sequence “EYSGVTSAHCNLCLLSSSDSRASASQVAGITAPATTPG” encoded by the inserted Alu-type nucleotide fragment.

The second mutation relates to the introduction of an early stop codon into the first quarter of the coding sequence, at the level of exon 13 of the ABC1 gene, leading to the production of a truncated polypeptide having 574 amino acids having the sequence SEQ ID NO 141. In addition, the deletion of the G base induces a change in the reading frame leading to a protein whose COOH-terminal end is not found in the amino acid sequence of the normal ABC1 polypeptide. This is the COOH-terminal sequence “RAPRRKLVSICNRCPIPVTLMTSFCG” of the mutated ABC1 polypeptide having the sequence SEQ ID NO 141.

These two polypeptides are useful in particular for the preparation of antibodies specifically recognizing them. Such antibodies constitute means of detection of the production of these mutated ABC1 polypeptides in a sample obtained from a subject to be tested, preferably a patient having symptoms characteristic of a deficiency in the reverse transport of cholesterol, and most preferably in a patient having the symptoms characteristic of Tangier disease.

According to another aspect, the invention therefore relates to a polypeptide comprising an amino acid sequence SEQ ID NO 140.

According to another aspect, the invention relates to a polypeptide comprising an amino acid sequence SEQ ID NO 141.

The invention also relates to a polypeptide comprising an amino acid sequence having at least 80% amino acid identity with an amino acid sequence chosen from the group consisting of the peptides having the sequences SEQ ID NO 140 and 141, or a peptide fragment thereof.

A first preferred peptide fragment will comprise at least 5 consecutive amino acids of the peptide fragment having the sequence “EYSGVTSAHCNLCLLSSSDSRASASQVAGITAPATTPG” contained in the mutated ABC1 polypeptide having the sequence SEQ ID NO 140.

A second preferred peptide fragment will comprise at least 5 consecutive amino acids of the peptide fragment having the sequence “RAPRRKLVSICNRCPIPVTLMTSFCG” contained in the mutated ABC1 polypeptide having the sequence SEQ ID NO 141.

Avantageously, a polypeptide having at least 85%, 90%, 95% or 99% amino acid identity with an amino acid sequence chosen from the group consisting of the peptides having the sequences SEQ ID NO 140 and 141, or a peptide fragment thereof, forms part of the invention.

Preferably, polypeptides according to the invention will have a length of 15, 18 or 20 to 25, 35, 40, 50, 70, 80, 100 or 200 consecutiuve amino acids of a nucleic acid according to the invention, in particular a polypeptide having an amino acid sequence chosen from the sequences SEQ ID No 140 and 141.

Alternatively, a polypeptide according to the invention will consist of and/or will comprise the fragments having a length of 15, 18, 20, 25, 35, 40, 50, 100 or 200 consecutive amino acids of a polypeptide according to the invention, more particularly of a polypeptide chosen from the sequences SEQ ID NO 140 and 141.

Generally, the polypeptides according to the invention are provided in an isolated or purified form.

The invention also relates to a method for the production of one of the polypetides having the sequences SEQ ID NO 140 and 141, or of a peptide fragment or of a variant thereof, said method comprising the steps of:

a) inserting a nucleic acid encoding said polypeptide into an appropriate vector;

b) culturing, in an appropriate culture medium, a previously transformed host cell or transfecting with the recombinant vector of step a);

c) recovering the conditioned culture medium or lysing the host cell, for example by sonication or by osmotic shock;

d) separating and purifying said polypeptide from said culture medium or alternatively from the cell lysates obtained in step c);

e) where appropriate, characterizing the recombinant polypeptide produced.

The peptides according to the invention may be characterized by binding to an immunoaffinity chromatography column on which the antibodies directed against this polypeptide or against a fragment or a variant thereof have been previously immobilized.

According to another aspect, a recombinant polypeptide according to the invention may be purified by passing over an appropriate series of chromatography columns, according to methods known to persons skilled in the art and described for example in F. Ausubel et al (1989).

A polypeptide according to the invention may also be prepared by conventional chemical synthesis techniques either in homogeneous solution or in solid phase.

By way of illustration, a polypeptide according to the invention may be prepared by the technique either in homogeneous solution described by Houben Weyl (1974) or the solid phase synthesis technique described by Merrifield (1965a; 1965b).

Polypeptides termed “homologous” to any one of the polypeptides having the amino acid sequences SEQ ID NO 140 and 141, or their fragments or variants, also form part of the invention.

Such homologous polypeptides have amino acid sequences possessing one or more substitutions of an amino acid by an equivalent amino acid, relative to the reference polypeptides.

Equivalent amino acid according to the present invention will be understood to mean for example replacement of a residue in the L form by a residue in the D form or the replacement of a glutamic acid (E) by a pyro-glutamic acid according to techniques well known to persons skilled in the art. By way of illustration, the synthesis of peptide containing at least one residue in the D form is described by Koch (1977).

According to another aspect, two amino acids belonging to the same class, that is to say two uncharged polar, nonpolar, basic or acidic amino acids, are also considered as equivalent amino acids.

Polypeptides comprising at least one nonpeptide bond such as a retro-inverse bond (NHCO), a carba bond (CH₂CH₂) or a ketomethylene bond (CO—CH₂) also form part of the invention.

Preferably, the polypeptides according to the invention comprising one or more additions, deletions, substitutions of at least one amino acid will retain their capacity to be recognized by antibodies directed against the nonmodified polypeptides.

Antibodies

The mutated ABC1 polypeptides according to the invention, in particular the polypeptides having the amino acid sequences SEQ ID NO 140-141] or the fragments thereof as well as the homologous peptides may be used for the preparation of antibodies, in particular for detecting the production of altered forms of the ABC1 polypeptide in a patient.

A first preferred antibody according to the invention is directed against a peptide fragment comprising at least 5 consecutive amino acids of the peptide fragment having the sequence “EYSGVTSAHCNLCLLSSSDSRASASQVAGITAPATTPG” contained in the mutated ABC1 polypeptide having the sequence SEQ ID NO 140.

A second preferred antibody according to the invention is directed against a peptide fragment comprising at least 5 consecutive amino acids of the peptide fragment having the sequence “RAPRRKLVSICNRCPIPVTLMTSFCG” contained in the mutated ABC1 polypeptide having the sequence SEQ ID NO 141.

“Antibody” for the purposes of the present invention will be understood to mean in particular polyclonal or monoclonal antibodies or fragments (for example the F (ab)′₂ and Fab fragments) or any polypeptide comprising a domain of the initial antibody recognizing the target polypeptide or polypeptide fragment according to the invention.

Monoclonal antibodies may be prepared from hybridomas according to the technique described by Kohler and Milstein (1975).

The present invention also relates to antibodies directed against a polypeptide as described above or a fragment or a variant thereof, as produced in the trioma technique or the hybridoma technique described by Kozbor et al. (1983).

The invention also relates to single-chain Fv antibody fragments (ScFv) as described in U.S. Pat. No. 4,946,778 or by Martineau et al. (1998).

The antibodies according to the invention also comprise antibody fragments obtained with the aid of phage libraries Ridder et al., (1995) or humanized antibodies Reinmann et al. (1997); Leger et al., (1997).

The antibody preparations according to the invention are useful in immunological detection tests intended for the identification of the presence and/or of the quantity of antigens present in a sample.

An antibody according to the invention may comprise, in addition, a detectable marker which is isotopic or nonisotopic, for example fluorescent, or may be coupled to a molecule such as biotin, according to techniques well known to persons skilled in the art.

Thus, the subject of the mention is, in addition, a method of detecting the presence of a polypeptide in accordance with the invention in a sample, said method comprising the steps of:

a) bringing the sample to be tested into contact with an antibody as described above;

b) detecting the antigen/antibody complex formed.

The invention also relates to a box or kit for diagnosis or for detecting the presence of a polypeptide in accordance with the invention in a sample, said box comprising:

a) an antibody as defined above;

b) a reagent allowing the detection of the antigen/antibody complexes formed.

Pharmaceutical Compositions and Therapeutic Methods of Treatment

The invention also relates to pharmaceutical compositions intended for the prevention or treatment of a deficiency in the metabolism of cholesterol such as atherosclerosis, particularly in the transport of cholesterol, and still more particularly in the reverse transport of cholesterol, characterized in that they comprise a therapeutically effective quantity of a polynucleotide capable of giving rise to the production of an effective quantity of the normal ABC1 polypeptide, in particular of the polypeptide having the sequence SEQ iD NO 139.

The subject of the invention is, in addition, pharmaceutical compositions intended for the prevention or treatment of a deficiency in the metabolism of cholesterol such as atherosclerosis, particularly in the transport of cholesterol, and still more particularly in the reverse transport of cholesterol, characterized in that they comprise a therapeutically effective quantity of the normal ABC1 polypeptide, in particular of the polypeptide having the sequence SEQ ID NO 139.

Such pharmaceutical compositions will be advantageously suitable for the administration, for example by the parenteral route, of a quantity of the ABC1 polypeptide ranging from 1 μg/kg/day to 10 mg/kg/day, preferably at least 0.01 mg/kg/day and most preferably between 0.01 and 1 mg/kg/day.

The pharmaceutical compositions according to the invention may be equally well administered by the oral, rectal, parenteral, intravenous, subcutaneous or intradermal route.

The invention also relates to the use of the ABC1 polypeptide having the sequence SEQ ID NO 139 for the manufacture of a medicament intended for the prevention of Atherosclerosis in various forms or more particularly for the treatment of subjects affected by a dysfunction in the reverse transport of cholesterol.

The invention finally relates to a pharmaceutical composition for the prevention or treatment of subjects affected by a dysfunction in the reverse transport of cholesterol, comprising a therapeutically effective quantity of the polypeptide having the sequence SEQ ID NO 139.

According to another aspect, the subject of the invention is also a preventive or curative therapeutic method of treating diseases caused by a deficiency in the metabolism of cholesterol, more particularly in the transport of cholesterol and still more particularly in the reverse transport of cholesterol, such a method comprising a step in which there is administered to a patient a polynucleotide capable of giving rise to the expression of the ABC1 polypeptide in said patient, said polynucleotide being, where appropriate, combined with one or more physiologically compatible vehicles and/or excipients.

Preferably, a pharmaceutical composition comprising a polynucleotide, as defined above, will be administered to the patient.

According to yet another aspect, the subject of the invention is also a preventive or curative therapeutic method of treating diseases caused by a deficiency in the metabolism of cholesterol, more particularly in the transport of cholesterol and still more particularly in the reverse transport of cholesterol, such a method comprising a step in which there is administered to a patient a therapeutically effective quantity of the ABC1 polypeptide in said patient, said polypeptide being, where appropriate, combined with one or more physiologically compatible vehicles and/or excipients.

Preferably, a pharmaceutical composition comprising a polypeptide, as defined above, will be administered to the patient.

Methods of Screening an Agonist or Antagonist Compound for the ABC1 Polypeptide

According to another aspect, the invention also relates to various methods of screening compounds for therapeutic use which are useful in the treatment of diseases due to a deficiency in the metabolism of cholesterol, particularly in the transport of cholesterol, still more particularly in the reverse transport of cholesterol, such as Tangier disease, or more generally FHD-type conditions.

The invention therefore also relates to the use of the ABC1 polypeptide, or of cells expressing the ABC1 polypeptide, for screening active ingredients for the prevention or treatment of diseases resulting from a dysfunction in the reverse transport of cholesterol.

The catalytic sites and oligopeptide or immunogenic fragments of the ABC1 polypeptide can serve for screening product libraries by a whole range of existing techniques. The fragment used in this type of screening may be free in solution, bound to a solid support, at the cell surface or in the cell. The formation of the binding complexes between the ABC1 fragments and the tested agent can then be measured.

Another product screening technique which may be used in high-flux screenings giving access to products having affinity for the protein of interest is described in application WO84/03564. In this method, applied to the ABC1 protein, various products are synthesized on a solid surface. These products react with the ABC1 protein or fragments thereof and the complex is washed. The products binding the ABC1 protein are then detected by methods known to persons skilled in the art. Nonneutralizing antibodies can also be used to capture a peptide and immobilize it on a support.

Another possibility is to use a product screening using the ABC1 neutralizing antibody competition, the ABC1 protein and a product potentially binding the ABC1 protein. In this manner, the antibodies may be used to detect the presence of peptide having common antigenic units with ABC1.

In the products to be evaluated and making it possible to increase the ABC1 activity, there may be mentioned in particular the kinase-specific ATP homologs involved in the activation of the molecules as well as phosphatases which may be able to avoid the dephosphorylation resulting from said kinases. There may be mentioned in particular inhibitors of of the phosphodiesterase (PDE) theophylline and 3-isobutyl-1-methylxanthine type or the adenylcyclase forskolin activators.

Accordingly, we claim in this invention the use of any method of screening products based on the method of translocation of cholesterol (see Example 17) between the membranes or vesicles, this being in all synthetic or cellular types, that is to say of mammals, insects, bacteria or yeasts expressing constitutively or having incorporated the human ABC1 sequence. To this effect, labeled lipid analogs may be used.

Likewise, it has been described that the ABC1 protein allowed anion transport (Becq et al. Journal of Biological Chemistry vol 272, No. 5 pages 2695-2699, 1997 and Yamon et al. Blood vol 90, No. 8 pages 2911-2915, 1997) and this transport was activated by phosphatase inhibitors such as okadaic acid and orthovanadate as well as part of the elevation of cAMP by agents such as forskolin. We claim the use of this system for screening molecules modulating the activity of the ABC1 protein (see Example 18).

Yamon et al (Blood vol 90, No. 8 pages 2911-2915, 1997) have demonstrated that the mouse ABC1 protein was involved in the secretion of a proinflammatory cytokine IL-1 beta in mouse peritoneal macrophages. It is therefore also possible to provide a method of screening products modulating the activity of the ABC1 protein by determining the release of IL-1beta from any cell type expressing two proteins (see Example 19).

Furthermore, knowing that the disruption of numerous transporters have been described (van, den Hazel. H., H. Pichler, V. M. M. do, E. Leitner, A. Goffeau, and G. Daum. 1999. PDR16 and PDR17, two homologous genes of Saccharomyces cerevisiae, affect lipid biosynthesis and resistance to multiple drugs. J. Biol. Chem. 274 (4):193441), it is possible to think of using cellular mutants having a characteristic phenotype and to complement the function thereof with ABC1 and to use the whole for screening purposes.

The invention also relates to a method of screening a compound active on the metabolism of cholesterol, an agonist or antagonist of the ABC1 polypeptide, said method comprising the following steps:

a) preparing membrane vesicles containing the ABC1 polypeptide and a lipid substrate comprising a detectable marker;

b) incubating the vesicles obtained in step a) with an agonist or antagonist candidate compound;

c) qualitatively and/or quantitatively measuring the release of the lipid substrate comprising a detectable marker;

d) comparing the measurement obtained in step b) with a measurement of the release of the labeled lipid substrate by vesicles which have not been previously incubated with the agonist or antagonist candidate compound.

According to a first aspect of the above screening method, the membrane vesicles are synthetic lipid vesicles, which may be prepared according to techniques well known to persons skilled in the art. According to this particular aspect, the ABC1 protein may be a recombinant ABC1 protein.

According to a second aspect, the membrane vesicles are vesicles of plasma membranes derived from cells expressing the ABC1 polypeptide. These may be cells naturally expressing the ABC1 polypeptide or cells transfected with a recombinant vector encoding the ABC1 polypeptide.

According to a third aspect of the above screening method, the lipid substrate is chosen from cholesterol or phosphatidyicholine.

According to a fourth aspect, the lipid substrate is radioactively labeled, for example with an isotope chosen from ³H or ¹²⁵I.

According to a fifth aspect, the lipid substrate is labeled with a fluorescent compound, such as NBD or pyrene.

According to a sixth aspect, the membrane vesicles comprising the labeled lipid substrate and the ABC1 polypeptide are immobilized at the surface of a solid support prior to step b).

According to a seventh aspect, the measurement of the fluorescence or of the radioactivity released by the vesicles is the direct reflection of the activity of lipid substrate transport by the ABC1 polypeptide.

The invention also relates to a method of screening a compound active on the metabolism of cholesterol, an agonist or antagonist of the ABC1 polypeptide, said method comprising the following steps:

a) obtaining cells, for example a cell line, expressing naturally or after transfection the ABC1 polypeptide;

b) incubating the cells of step a) in the presence of an anion labeled with a detectable marker,

c) washing the cells of step b) in order to remove the excess of the labeled anion which has not penetrated into these cells;

d) incubating the cells obtained in step c) with an agonist or antagonist candidate compound for the ABC1 polypeptide;

e) measuring the efflux of the labeled anion;

f) comparing the value of the efflux of the labeled anion determined in step e) with the value of the efflux of the labeled anion measured with cells which have not been previously incubated in the presence of the agonist or antagonist candidate compound for the ABC1 polypeptide.

According to a first aspect of the above screening method, the cells used are cells naturally expressing the ABC1 polypeptide. They may be human monocytes in primary culture, purified from a population of human blood mononuclear cells. They may also be human monocytic cell lines, such as the monocytic leukemia line THP1.

According to a second aspect, the cells used in the screening method described above may be cells not naturally expressing, or alternatively expressing at a low level, the ABC1 polypeptide, said cells being transfected with a recombinant vector according to the invention capable of directing the expression of the ABC1 polypeptide.

According to a third aspect, the cells may be cells having a natural deficiency in anion transport, or cells pretreated with one or more anion channel inhibitors such as Verapamil™ or tetraethylammonium.

According to a fourth aspect of said screening method, the anion is a radioactively labeled iodide, such as the salts K¹²⁵I or Na¹²⁵I.

According to a fifth aspect, the measurement of the efflux of the labeled anion is determined periodically over time during the experiment, thus making it possible to also establish a kinetic measurement of this efflux.

According to a sixth aspect, the value of the efflux of the labeled anion is determined by measuring the quantity of labeled anion present at a given time in the cell culture supernatant.

According to a seventh aspect, the value of the efflux of the labeled anion is determined as the proportion of radioactivity found in the cell culture supernatant relative to the total radioactivity corresponding to the sum of the radioactivity found in the cell lysates and the radioactivity found in the cell culture supernatant.

The subject of the invention is also a method of screening a compound active on the metabolism of cholesterol, an agonist or antagonist of the ABC1 polypeptide, said method comprising the following steps:

a) culturing cells of a human monocytic line in an appropriate culture medium, in the presence of purified human albumin;

b) incubating the cells of step a) simultaneously in the presence of a compound stimulating the production of IL-1 beta and of the agonist or antagonist candidate compound;

c) incubating the cells obtained in step b) in the presence of an appropriate concentration of ATP;

d) measuring IL-1 beta released into the cell culture supernatant.

e) comparing the value of the release of the IL-1 beta obtained in step d) with the value of the IL-1 beta released into the culture supernatant of cells which have not been previously incubated in the presence of the agonist or antagonist candidate compound.

According to a first aspect of the screening method described above, the cells used belong to the human leukemic monocytic line THP1.

According to a second aspect of the screening method, the compound stimulating the production of IL-1 beta is a lipopolysaccharide. According to a third aspect of said method, the production of IL-1 alpha, IL-6 and TNF alpha by these cells is also qualitatively and/or quantitatively determined.

According to a fourth aspect, the level of expression of the messenger RNA encoding IL-1 beta is also determined.

The invention is illustrated, without being limited as a result, by the following figures and examples:

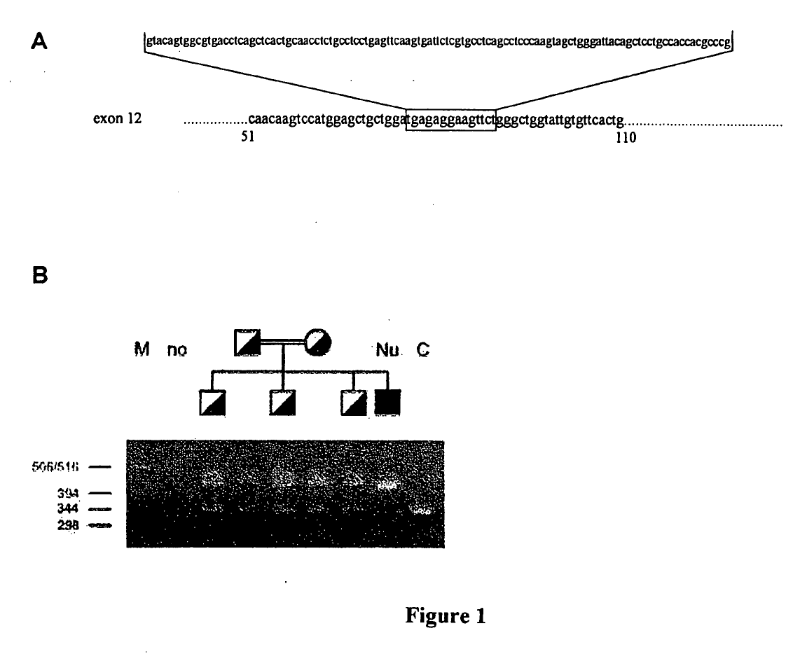

FIG. 1 illustrates the segregation of the mutation by insertion of an Alu sequence into exon 12 of the ABC1 gene. The insertion-deletion in exon 12 of the ABC1 gene constitutes a deletion of 14 nucleotides and of an insertion of 110 nucleotides as represented in FIG. 1A. FIG. 1B represents the Nu pedigree and the size of the DNA fragments obtained for each of the patients after PCR amplification of exon 12. Lane M corresponds to the mobility markers (Gibco BRL). Lane C corresponds to a control DNA.

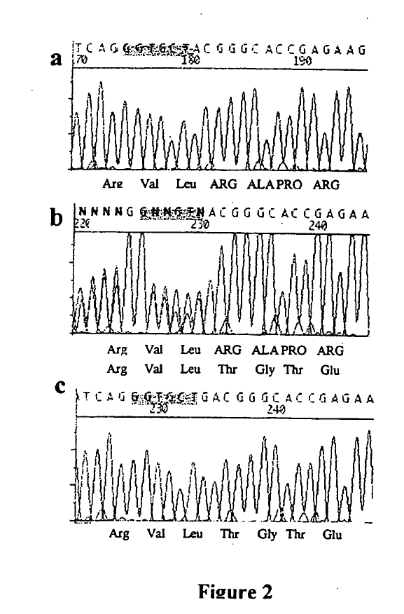

FIG. 2 illustrates the mutation by deletion of a single nucleotide in exon 13 of the ABC1 gene. The sequence of the complementary strand was obtained and is represented from 3′ to 5′. The sequence encoding amino acids 546 to 552 of the ABC1 polypeptide is represented for different patients of the family studied, respectively for a homozygous (FIG. 2-a), heterozygous (FIG. 2-b) and nonaffected (FIG. 2-c) individual. The sequence of FIG. 2-a was found in three homozygous individuals, the sequence of FIG. 2-b was found in five heterozygous individuals and the sequence of FIG. 2-c was found in four nonaffected individuals.

### EXAMPLES

### Example 1

### Tissue Distribution of the Transcripts of the ABC1 Gene According to the Invention

The profile of expression of the polynucleotides according to the present invention is determined according to the protocols for PCR-coupled reverse transcription and Northern blot analysis described in particular by Sambrook et al. (ref. CSH Sambrook, J., Fritsch, E. F., and Maniatis, T. (1989). “Molecular Cloning: A Laboratory Manual,” 2nd ed., Cold Spring Harbor Laboratory Press, Cold Spring Harbor, N.Y.).

For example, in the case of an analysis by reverse transcription, a pair of primers synthesized from the complete DNA of the human ABC1 gene having the sequence SEQ ID NO 91 is used to detect the corresponding cDNA.

The polymerase chain reaction (PCR) is carried out on cDNA templates corresponding to retrotranscribed polyA⁺ mRNAs (Clontech). The reverse transcription to cDNA is carried out with the enzyme SUPERSCRIPT II (GibcoBRL, Life Technologies) according to the conditions described by the manufacturer. The polymerase chain reaction is carried out according to standard conditions, in 20 μl of reaction mixture with 25 ng of cDNA preparation. The reaction mixture is composed of 400 μM of each of the dNTPs, 2 units of Thermus aquaticus (Taq) DNA polymerase (Ampli Taq Gold; Perkin Elmer), 0.5 μM of each primer, 2.5 mM MgCl2, and PCR buffer. Thirty four PCR cycles (denaturing 30 s at 94° C., annealing of 30 s divided up as follows during the 34 cycles: 64° C. 2 cycles, 61° C. 2 cycles, 58° C. 2 cycles and 55° C. 28 cycles and an extension of one minute per kilobase at 72° C.) are carried out after a first step of denaturing at 94° C. for 10 min in a Perkin Elmer 9700 thermocycler. The PCR reactions are visualized on agarose gel by electrophoresis. The cDNA fragments obtained may be used as probes for a Northern blot analysis and may also be used for the exact determination of the polynucleotide sequence.

In the case of a Northern Blot analysis, a cDNA probe produced as described above is labeled with ³²P by means of the DNA labeling system High Prime (Boehringer) according to the instructions indicated by the manufacturer. After labeling, the probe is purified on a Sephadex G50 microcolumn (Pharmacia) according to the instructions indicated by the manufacturer. The labeled and purified probe is then used for the detection of the expression of the mRNAs in various tissues.

The Northern blot containing samples of RNA of different human tissues (Multiple Tissue Northern, MTN, Clontech) Blot 2, reference 77759-1) is hybridized with the labeled probe.

The protocol followed for the hybridizations and washes may be either directly as that described by the manufacturer (Instruction manual PT1200-1) or an adaptation of this protocol using methods known to persons skilled in the art and described for example in F. Ausubel et al (1999). It is thus possible to vary, for example, the prehybridization and hybridization temperatures in the presence of formamide.

For example, it may be possible to use the following protocol:

1—Membrane Competition and Prehybridization:

Mix: 40 μl salmon sperm DNA (10 mg/ml)+40 μl human placental DNA (10 mg/ml)

Denature for 5 min at 96° C., then immerse the mixture in ice.

Remove the 2×SSC and pour 4 ml of formamide mix In the hybridization tube containing the membranes.

Add the mixture of the two denatured DNAs.

Incubation at 42° C. for 5 to 6 hours, with rotation.

2—Labeled Probe Competition:

Add to the labeled and purified probe 10 to 50 μl Cot I DNA, depending on the quantity of repeat sequences.

Denature for 7 to 10 min at 95° C.

Incubate at 65° C. for 2 to 5 hours.

3—Hybridization:

Remove the prehybridization mix, Mix 40 μl salmon sperm DNA+40 μl human placental DNA; denature for 5 min at 96° C., then immerse in ice.

Add to the hybridization tube 4 ml of formamide mix, the mixture of the two DNAs and the denatured labeled probe/Cot I DNA.

Incubate 15 to 20 hours at 42° C., with rotation.

4—Washes:

One wash at room temperature in 2×SSC, to rinse.

Twice 5 minutes at room temperature 2×SSC and 0.1% SDS at 65° C.

Twice 15 minutes at 65° C. 1×SSC and 0.1% SDS at 65° C.

After hybridization and washing, the blot is analyzed after overnight exposure in contact with a phosphorus screen revealed with the aid of Storm (Molecular Dynamics, Sunnyvale, Calif.).

### Example 2

### Production of the Complete cDNA of the ABC1 Gene

The sequence of the 3′-UTR region of the cDNA of the human ABC1 gene was identified by searching in databases.

An iterative screening of a database of EST sequences (“Genbank mouse human subdivision EST, v.111”) was carried out with the aid of the BLAST software.

Oligonucleotide primers were synthesized from the partial consensus sequence derived from the EST sequences, in order to amplify by an RT-PCR reaction the 3′ end of the cDNA of the human ABC1 gene, and then to determine the sequence thereof.

The oligonucleotide primers used are the following:

| | | |
|---|---|---|
| 1. | 5′-AAACCAGACAGTAGTGGACG-3′, | (SEQ ID NO 142) |
| 2. | 5′-GTTACTGCCACCAGAACAGC-3′, | (SEQ ID NO 143) |
| 3. | 5′-TGATAAGCTGTTCTGGTGGC-3′, | (SEQ ID NO 144) |
| 4. | 5′-CTTGGCTTTTGCATTGTTGC-3′, | (SEQ ID NO 145) |
| 5. | 5′-CAATGCAAAAGCCAAGAAAG-3′, | (SEQ ID NO 146) |
| 6. | 5′-TGCAACGATGCCATATCAC-3′, | (SEQ ID NO 147) |
| 7. | 5′-CAACTCCTTACTTCGGTTCCTC-3′, | (SEQ ID NO 148) |
| 8. | 5′-GTTTTCTGAGGTGTCCCAAAG-3′ | (SEQ ID NO 149) |

The reverse transcription of the poly(A)+ mRNA from brain, fetal brain, heart, uterus and placenta tissues (Libraries marketed by the company Clontech) was carried out by extension with the aid of oligodT primers using the Superscript™ kit (marketed by the company Life Technologies Inc.), according to the manufacturer's instructions.

In each experiment, it was possible to exclude the presence of contaminating DNA because of the absence of PCR-amplified polynucleotides in the samples not containing reverse transcriptase.

A PCR reaction was carried out on the products which have been subjected or otherwise to a first step of reverse transcription under the following conditions:

400 μM dNTP, 2 Units of Taq DNA polymerase (Thermus aquaticus, Ampli Taq Gold, marketed by the company Perkin Elmer), 0.5 μM of each of the primers, 2.5 mM of Mg Cl₂, the whole being present in a PCR buffer also containing 50 ng of DNA and about 25 ng of cDNA.

The PCR reaction was carried out for 30 cycles in a thermocycler apparatus (“Perkin Elmer 9700 Thermal Cycler”) in 96-well microplates.

After an initial denaturation at 94° C. for 10 minutes, each cycle was carried out in the following manner:

step of denaturation at 94° C. for 30 seconds; step of annealing for 30 seconds (at 64° C. for 2 cycles, at 61° C. for 2 cycles, at 58° C. for 2 cycles and at 55° C. for 28 cycles).

step of extension for a period corresponding to 1 minute per kilobase.

The PCR reaction was stopped by a final extension step of 7 minutes.

Various other approaches may be used to isolate the cDNA corresponding to the complete cDNA of ABC1.

For example, a complete clone may be directly isolated by hybridization by screening a cDNA library by means of a polynucleotide probe specific for the sequence of the gene of interest.

In particular, a specific probe of 3040 nucleotides is synthesized using a synthesizer of the Applied Biosystem/Perkin Elmer trademark depending on the chosen sequence.

The oligonucleotide obtained is radiolabeled, for example with [γ-³²P]ATP using T4 polynucleotide kinase and is purified according to the customary methods (e.g Maniatis et al. Molecular cloning: A Laboratory Manual, Cold Spring Harbor Press, Cold Spring, N.Y. 1982 or F. Ausubel et al. (Current Protocols in Molecular Biology, J. Wiley and Sons Eds, 1989).

The clone library containing the cDNA which it is desired to screen is established on a culture medium in a Petri dish (1.5% agar) containing the appropriate antibiotics according to the customary methods cited above (F. Ausubel et al.). The colonies thus produced after incubation are transferred on nitrocellulose filters and screened by means of the radiolabeled nucleotide probe, according to the customary methods and the colonies hybridizing with the probe are isolated and subcloned.

The DNA of the clones thus identified is prepared and analyzed by sequencing. The clones containing the fragments corresponding to the complete cDNA are purified and recloned into the vector pcDNA3 according to the protocols known to persons skilled in the art and presented for example in F. Ausubel et al (1989).

Various methods are known for identifying the 5′ and 3′ ends of the cDNA corresponding to the genes described in the present application. These methods include but are not limited to hybridization cloning, to cloning using protocols similar or identical to 3′ or 5′ RACE-PCR (Rapid Amplification of cDNA End-PCR) which are well known to persons skilled in the art.

For example, it will be possible to use the kit marketed by the company Clontech (Marathon Ready™ cDNA kit, protocol identified by the reference PT1156-1), or alternatively a method similar to 5′RACE is available for characterizing the absent 5′ end of a cDNA (Fromont-Racine et al. Nucleic Acid Res. 21(7):1683-1684 (1993)). Briefly, an RNA oligonucleotide is ligated to the 5′ end of an mRNA population. After retrotranscription to cDNA, a set of primers specific respectively for the adaptor ligated in 5′ and for a sequence situated in 3′ of the gene of interest is used in PCR to amplify the 5′ portion of the desired cDNA. The amplified fragment is then used to reconstruct the complete cDNA.

### Example 3

### Analysis of the Gene Expression Profile for Tangier Disease

The verification of the impairment of the level of expression of the ABC1 gene causing the Tangier cellular phenotype may be determined by hybridizing these sequences with probes corresponding to the mRNAs obtained from fibroblasts of subjects suffering or otherwise from the disease, according to the methods described below:

1. Preparation of the Total RNAs, of the poly(A)⁺ mRNAs and of cDNA Probes

The total RNAs are obtained from cell cultures of the fibroblasts of normal-subjects or subjects suffering from Tangier disease by the guanidine isothiocyanate method (Chomczynski & Sacchi, 1987). The poly(A)⁺ mRNAs are obtained by affinity chromatography on oligo(dT)-cellulose columns (Sambrook et al., 1989) and the cDNAs used as probes are obtained by RT-PCR (DeRisi et al., 1997) with oligonucleotides labeled with a fluorescent product (Amersham Pharmacia Biotech CyDye™).

2. Hydridization and Detection of the Expression Levels

The glass membranes containing the sequences presented in this patent application, corresponding to the Tangier gene are hybridized with the cDNA probes obtained from fibroblasts (lyer et al., 1999). The use of the Amersham/molecular Dynamics system (Avalanche Microscanner™) allows the quantification of the expressions of the products of sequences on healthy or affected cell types.

### Example 4

### Construction of the Expression Vector Containing the Complete cDNA of ABC1 in Mammalian Cells

The ABC1 gene may be expressed in mammalian cells. A typical eukaryotic expression vector contains a promoter which allows the initiation of the transcription of the mRNA, a sequence encoding the protein, and the signals required for the termination of the transcription and for the polyadenylation of the transcript. It also contains additional signals such as enhancers, the Kozak sequence and sequences necessary for the splicing of the mRNA. An effective transcription is obtained with the early and late elements of the SV40 virus promoters, the retroviral LTRs or the CMV virus early promoter. However, cellular elements such as the actine promoter may also be used. Many expression vectors may be used to carry out the present invention such as the vector pcDNA3.

### Example 5

### Production of Normal and Mutated ABC1 Polypeptides

The normal ABC1 polypeptide encoded by the complete cDNA of ABC1 whose isolation is described in Example 2 (cloning of the complete cDNA), or the mutated ABC1 polypeptides whose complete cDNA may also be obtained according to the techniques described in Example 2, may be easily produced in a bacterial or insect cell expression system using the baculovirus vectors or in mammalian cells with or without the vaccinia virus vectors. All the methods are now widely described and are known to persons skilled in the art. A detailed description thereof will be found for example in F. Ausubel et al. (1989).

### Example 6

### Production of an Antibody Directed Against One of the Mutated ABC1 Polypeptides

The antibodies in the present invention may be prepared by various methods (Current Protocols In Molecular Biology Volume 1 edited by Frederick M. Ausubel, Roger Brent, Robert E. Kingston, David D. Moore, J. G. Seidman, John A. Smith, Kevin Struhl—Massachusetts General Hospital Harvard Medical School, chapter 11). For example, the cells expressing a polypeptide of the present invention are injected into an animal in order to induce the production of serum containing the antibodies. In one of the methods described, the proteins are prepared and purified so as to avoid contaminations. Such a preparation is then introduced into the animal with the aim of producing polyclonal antisera having a higher activity.

In the preferred method, the antibodies of the present invention are monoclonal antibodies. Such monoclonal antibodies may be prepared using the hybridoma technique (Köhler et al, Nature 256:495 (1975); Köhler et al, Eur. J. Immunol. 6:511 (1976); Köhler et al, Eur. J. Immunol. 6:292 (1976); Hammeling et al., in: Monoclonal Antibodies and T-Cell Hybridomas, Elsevier, N.Y, pp. 563-681 51981). In general, such methods involve immunizing the animal (preferably a mouse) with a polypeptide or better still with a cell expressing the polypeptide. These cells may be cultured in a suitable tissue culture medium. However, it is preferable to culture the cells in an Eagle medium (modified Earle) supplemented with 10% fetal bovine serum (inactivated at 56° C.) and supplemented with about 10 g/l of nonessential amino acids, 1000 U/ml of penicillin and about 100 μg/ml of streptomycin.

The splenocytes of these mice are extracted and fused with a suitable myeloma cell line However, it is preferable to use the parental myeloma cell line (SP2O) available from the ATCC. After fusion, the resulting hybridoma cells are selectively maintained in HAT medium and then cloned by limiting dilution as described by Wands et al. (Gastroenterology 80:225-232 (1981)). The hybridoma cells obtained after such a selection are tested in order to identify the clones secreting antibodies capable of binding to the polypeptide.

Moreover, other antibodies capable of binding to the polypeptide may be produced according to a 2-stage procedure using anti-idiotype antibodies such a method is based on the fact that the antibodies are themselves antigens and consequently it is possible to obtain an antibody recognizing another antibody. According to this method, the antibodies specific for the protein are used to immunize an animal, preferably a mouse. The splenocytes of this animal are then used to produce hybridoma cells, and the latter are screened in order to identify the clones which produce an antibody whose capacity to bind to the specific antibody-protein complex may be blocked by the polypeptide. These antibodies may be used to immunize an animal in order to induce the formation of antibodies specific for the protein in a large quantity.

It would be advantageous if Fab and F(ab′)2 and the other fragments of the antibodies of the present invention can be used according to the methods described here. Such fragments are typically produced by proteolytic cleavage with the aid of enzymes such as Papain (in order to produce the Fab fragments) or Pepsin (in order to produce the F(ab′)2 fragments). Otherwise, the secreted fragments recognizing the protein may be produced by applying the recombinant DNA or synthetic chemistry technology.

For the in vivo use of antibodies in humans, it would be preferable to use “humanized” chimeric monoclonal antibodies. Such antibodies may be produced using genetic constructs derived from hybridoma cells producing the monoclonal antibodies described above. The methods for producing the chimeric antibodies are known to persons skilled in the art (for a review, see: Morrison, Science 229:1202 (1985); Oi et al., Biotechnique 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al; Nature 312:643 (1984); Neuberger et al., Nature 314: 268 (1985)).

### Example 7

### Correction of the Cellular Phenotype of the Tangier Disease

The Tangier disease is characterized by an accelerated catabolism of the high-density lipoprotein (HDL) particles and an accumulation of cholesterol in the tissues. In particular, the fibroblasts of the skin of patients suffering from Tangier disease have a reduced capacity to eliminate their cholesterol content by the process of efflux of cholesterol carried out by apolipoprotein A-I (apoA-I), the major protein of the HDLs (Francis et al., 1995). This characteristic corresponding to a loss of function is also found in other fibroblast cells of patients suffering from familial HDL deficiency (Marcil et al., 1999).

The correction of the phenotype of the Tangier fibroblasts can be carried out by the transfection of the complete cDNA of ABC1 according to the invention, into said cells. The cDNA is inserted into an expression vector which is then transfected according to the methods described below:

1. Preparation of the Fibroblast Cultures of Normal Subjects and of Subjects Suffering from Tangier Disease

The primary fibroblasts of human skin are obtained by culturing a skin biopsy obtained from the forearm. These biopsies are performed on patients suffering from Tangier disease having the clinical and biochemical features of the “homozygotes”, that is to say orange-colored tonsils, plasma concentrations of apoA-1 and of cholesterol-HDL less than the 5^{th} percentile. The normal fibroblast lines are obtained from the American Type Culture Collection (Rockville, Md.). The fibroblasts are cultured in an EMMEM (Eagle-modified minimium essential medium; GIBCO) medium supplemented with 10% fetal calf serum, 2 mM glutamine, 100 IU/ml of penicillin and 100 μg/ml of steptomycin (medium designated EMMEM10). In order to carry out the study of the efflux of cholesterol, these cells are preloaded with cholesterol by incubating for 24 hours with 50 μg/ml of cholesterol in the medium described above without calf serum but containing 2 mg/ml of bovine albumin (BSA, fraction V).

2. Study of the Efflux of Cholesterol

The fibroblasts preloaded with cholesterol at confluence on 24-well plates are incubated in the EMMEM10 medium and 1 μCi/ml of 1,2-³H-cholesterol (50 Ci/mmol; Dupont; Wilmington, Del.) for 48 hours. About 100,000 counts per minute are obtained per well or 1000 counts per minute and per pg of cellular protein. The cells are washed three times with EMMEM/BSA medium, and incubated with this medium for 24 hours before transfecting the gene of interest and starting the efflux by adding 10 μg/ml of proteoliposome containing apoA-1 in EMMEM/BSA medium. These proteoliposomes are prepared by sonication of phosphatidylcholine and purified human apoA-I (Jonas, 1986). The cell transfection is carried out by the calcium phosphate precipitation technique (Sambrook et at., 1989). After the period of efflux, in general 20 hours, the medium is collected, centrifuged (1000 g, 5 min), and the radioactivity determined by liquid scintillation counting. The residual radioactivity in the cells is also determined overnight after extraction of the lipids in isopropanol. The percentage efflux is calculated by dividing the radioactivity measured in the supernatant by the sum of the radioactivities measured, in the supernatant and the cellular extract. An internal standard is prepared by transfection of a marker gene and incubation for 24 hours with an EMMEM/IBSA medium without proteoliposome containing apoA-I. The efflux of cellular cholesterol from normal fibroblasts transfected with a control gene correspond to 6+2% whereas that obtained from fibroblasts suffering from Tangier disease and transfected with this control gene is less than 1%. On the other hand, the transfection of the fibroblasts suffering from Tangier disease with a plasmid containing the complete cDNA or the genomic DNA for ABC1 according to the invention could make it possible to restore the capacity of these cells to eliminate their excess of cholesterol at a level corresponding to that of normal fibroblasts.

### Example 8

### Isolation and Characterization of Genomic Fragments of the Human ABC1 Gene

A fragment of about 3 kb of the human ABC1 cDNA was obtained from the cDNA clone designated “pf10” containing the first ATP-binding domain of ABC1, this cDNA clone being described in the article by Luciani et al. (1994).

This cDNA fragment obtained by digestion of the clone pf10 with the aid of the restriction endonuclease EcoRI, was isolated on an agarose gel after electrophoresis, then labeled with digoxigenin according to the manufacturer's instructions (kit marketed by Boehringer Mannheim, reference 1 585 614).

The labeled cDNA fragment was used to screen the LLNL (Lawrence Livermore National Labs) cosmid library of chromosome 9, immobilized on a Nylon™ filter.

Six positive clones were identified. For these six cosmids, the probe hybridized with single colonies.

A representative clone was isolated from each of these colonies.

The clones LLNLC 131J087 Q2 (designated here cos3a) and LLNLc 131O1165 Q2 (designated here cos6f) were analyzed in greater detail.

The clone cos3a was subcloned in the form of an EcoRI fragment into the vector Gen3zf(−) and sequenced at both ends using the Big Dye Terminator technology on an AB1377 type sequencer (Applied Biosystems, Perkin Elmer).

The clones containing distinct inserts (determined after sequencing of the ends of the various inserts or by determining the size thereof) which were too long to be completely sequenced with the aid of the primers hybridizing with the sequences of the vector, were analyzed more before by the technique of transposon insertion and then of sequencing with the aid of primers specific to the transposon (“GPS” system marketed by the company New England Biolabs).

In this manner, genomic sequences corresponding to the human ABC1 gene were isolated and characterized. These sequences were compared with human and mouse sequences identified by references in the databases making it possible to determine the intron-exon junctions.

### Example 9

### Determination of Polymorphisms/Mutations in the ABC1 Gene

The detection of polymorphisms or of mutations in the sequences of the transcripts or in the genomic sequence of the ABC1 gene may be carried out according to various protocols. The preferred method is direct sequencing. For patients from whom it is possible to obtain an mRNA preparation, the preferred method consists in preparing the cDNAs and sequencing them directly. For patients for whom only DNA is available, and in the case of a transcript where the structure of the corresponding gene is unknown or partially known, it is necessary to precisely determine its intron-exon structure as well as the genomic sequence of the corresponding gene. This therefore involves, in a first instance, isolating the genomic DNA BAC or cosmid clone(s) corresponding to the transcript studied according to the method described in Example 8, sequencing the insert of the corresponding clone(s) and determining the intron-exon structure by comparing the cDNA sequence to that of the genomic DNA obtained.

The technique of detection of mutations by direct sequencing consists in comparing the genomic sequences of the ABC1 gene obtained from homozygotes for the disease or from at least 8 individuals (4 individuals affected by the pathology studied and 4 individuals not affected). The sequence divergences constitute polymorphisms. All those modifying the amino acid sequence of the wild-type protein may be mutations capable of affecting the function of said protein which it is advantageous to consider more particularly for the study of cosegregation of the mutation and of the disease (denoted genotype-phenotype correlation) in the pedigree or in the studies of case/control association for the analysis of the sporadic cases

### Example 10

### Identification of a Causal Gene for a Disease Linked to a Deficiency in the Reverse Transport of Cholesterol by Causal Mutation or a Transcriptional Difference

Among the mutations identified according to the method described in Example 9, all those associated with the disease phenotype are capable of being causal. Validation of these results is made by sequencing the gene in all the affected individuals and their relations (whose DNA is available). Moreover, the carrying out of Northern blotting or RT-PCR, according to the method described in Example 1, using RNA specific to affected or nonaffected individuals makes it possible to detect notable variations in the level of expression of the gene studied, in particular in the absence of transcription of the gene.

### Example 11

### Identification of a Deletion of a Nucleotide in Exon 13 of the ABC1 Gene in Patients Suffering from TANGIER Disease

The analysis of mutations in the ABC1 gene was carried out on genomic DNA from several individuals belonging to a family of which several members suffer from Tangier disease with premature coronary disorders.

A deletion of one nucleotide was identified in exon 13 (DG 1764: Leu548Leu;575 End). This deletion introduces a stop codon at position 575 which makes it possible to predict a truncation of the ABC1 protein encoded by the mutated ABC1 gene, this truncation leading to the synthesis of a polypeptide deleted of a large portion of the normal amino acid sequence, and in particular of the two cassettes for binding to ATP.

A perfect correlation between the observation of the symptoms of the disease and the presence of this deletion of one nucleotide was found in the entire family (FIG. 1).

### Example 12

### Identification of an Insertion of a Segment of Nucleotides into Exon 12 of the ABC1 Gene

In another family in which several members suffer from Tangier disease, an insertion of 110 base pairs having the structure of a repeated nucleotide sequence of the Alu-sq type, accompanied by a deletion of 14 base pairs in exon 12, was observed (FIG. 2). This insertion/deletion mutation makes it possible to predict a deletion of 65 amino acids (DERKFW) as well as an insertion in phase of 38 amino acids (EYSGVTSAHCNLCLLSSSDSRASASQVAGITAPATTPG).

This mutation does not allow the synthesis of a normal ABC1 transport polypeptide. It is therefore possible to conclude that Tangier disease, in the individuals in this family, is caused by a deficiency in the ABC1 gene.

### Example 13

### Identification of Biallelic Polymorphisms in the ABC1 Gene

Primers for the amplification of the DNA of the patients were designed from nonrepetitive sequences of the intron DNA of the ABC1 gene, in such a way that an amplification of the intron-exon junctions as well as the bases essential for the formation of the secondary structure during the RNA splicing step are included in the amplified fragments.

The various pairs of primers specifically developed are presented in Table V.

The results found on the DNA from a family containing cases of Tangier disease without coronary complication are shown in Table IV.

The genomic DNA of the patients was amplified with the aid of the primers described above using Qiagen's Star Taq kit or the Supertaq kit, using the hybridization conditions and the amplification cycle conditions recommended by the manufacturer.

The amplified PCR products were then purified using a kit marketed by the company Qiagen, and then sequenced by the Big Dye Terminator method on an AB1377 sequencer (Applied Biosystems, Perkin Elmer).

### Example 14

### Identification of a Region of 1 cM on the 9q31 Locus Associated with Tangier Disease

A first linkage analysis was described in the article by Rust et al. (1998).

This article presented a linkage analysis on three families of patients suffering from Tangier disease and defined a candidate interval of 0.8 cM in 9q31.

The applicant has carried out a linkage study by including four additional families as well as additional markers identified by references in public databases in order to refine the candiate region to about 1 cM, with reference to the genetic map published by Généthon (Dib et al., 1996)

The results of linkage analysis presented below allowed the applicant to exclude from the candidate region the genomic segments respectively proximal (centromeric) and distal (telomeric) to the markers D9S271 and D9S1866.

The candidate region is therefore located between these two excluded markers.

An important piece of information which made it possible to refine the candidate region was obtained from the E1 portion of the pedigree described in the article by Rust et al. (1998). Indeed, it has been shown on the maternal chromosome, at the origin of the E121m portion, that the recombination event (crossing-over) already described in FIG. 2 of this article (between the markers D9S277 and D9S53), in fact ought to be located telomerically relative to the marker D9S271. The centromeric boundary of the candidate interval being situated between D9S271 and D9S277.

In two of the new families, respectively family “S1” and family “NU”, additional recombination events were observed. These recombination events made it possible to move the telomeric boundary of the candidate interval, from the marker D9S1677 (described in the article by Rust et al 1998) to the marker D9S1866.

The first pedigree, “S1”, was extended. The affected individuals exhibit a homozygous genotype for all the markers of the 8 cM region as well as for the more distant markers, located on either side of this region. One of the cousins of the individual S1, related to S1 by both parents (double consanguinity) has four children. Two of these children exhibit on their chromosome of paternal origin conservation of a large portion of the diseased haplotype (in the defined region of 8 cM). These two children also exhibit the typical characteristic of the heterozygous parents of the family suffering from Tangier disease, namely an HDL level which is half the level observed in patients not affected by the disease.

However, the character homozygous for the markers is no longer observed in the chromosomal region starting from the marker D9S1866 (which is heterozygous in these individuals), which made it possible to define D9S1866 as the telomeric boundary of the candidate region.

The same telomeric boundary was observed in the “Nu” family, in which one of the four children from parents who were first degree cousins, was a patient affected by homozygous Tangier disease.

A homozygosity for the markers on the entire candidate region was observed in this patient.

One of his brothers, a heterozygote (at the phenotype level), exhibits a recombination event (crossing-over) on one of the two chromosomes, such that this brother is homozygous (at the genetic level) for all the telomeric markers including the marker D9S1866, but heterozygous (at the genetic level) for the markers located in the region near the centromer.

As this patient did not exhibit a phenotype of a homozygous patient, it was possible to exclude the telomeric region including the marker D9S1866.

### Example 15

### Isolation and Characterization of the Human ABC1 Gene

A fragment of about 3 kb of the human ABC1 cDNA was obtained from a cDNA clone designated “pf10” containing the first ATP-binding domain of ABG1, this cDNA clone being described in the article by Luciani et al. (1994).

This cDNA fragment obtained by digestion of the clone pf10 with the aid of the restriction endonuclease EcoRI, was isolated on an agarose gel after electrophoresis, then labeled with digoxigenin according to the manufacturer's instructions (kit marketed by Boehringer Mannheim).

The labeled cDNA fragment was used to screen the LLNL cosmid library of chromosome 9, immobilized on a Nylon™ filter.

Six positive clones were identified. For these six cosmids, the probe hybridized with single colonies.

A representative clone was isloated from each of these colonies.

The clones LLNLC 131J087 Q2 (designated here cos3a) and LLNLc 131O1165 Q2 (designated here cos6f) were analyzed in greater detail.

The clone cos3a was subcloned in the form of an EcoRI fragment into the vector Gen3zf(−) and sequenced at both ends using the Big Dye Terminator technology on an A81377 type sequencer.

The clones containing distinct inserts (determined after sequencing of the ends of the various inserts or by determining the size thereof) which were too long to be completely sequenced with the aid of the primers hybridizing with the sequences of the vector, were analyzed more before by the technique of transposon insertion and then of sequencing with the aid of primers specific to the transposon (“GPS” system marketed by the company New England Biolabs).

In this manner, genomic sequences corresponding to the human ABC1 gene were isolated and characterized. These sequences were compared with human and mouse sequences identified by references in the databases.

The sequences of the intron-exon junctions were determined.

Primers for the amplification of the DNA of the patients were designed from nonrepetitive sequences of the intron DNA of the ABC1 gene, in such a way that an amplification of the intron-exon junctions as well as the bases essential for the formation of the secondary structure during the splicing step are included in the amplified fragments.

The genomic DNA of the patients was amplified with the aid of the primers described above using Qiagen's Star Taq kit or the Supertaq kit, using the hybridization conditions and the amplification cycle conditions recommended by the manufacturer.

The amplified PCR products were then purified using a kit marketed by the company Qiagen, and then sequenced by the Big Dye Terminator method on an ABI377 sequencer.

### Example 16

### Construction of Recombinant Vectors Containing a Polynucleotide Encoding the ABC1 Protein

I. Synthesis of of the Human ABC1 Gene.

Total RNA (500 ng) isolated from human placental tissue (Clontech, Palo Alto, Calif., USA) was used as source for the synthesis of the cDNA of the human ABC1 gene, using the system “Superscript one step RT-PCR (Life Technologies, Gaithersburg, Md., USA) and the oligonucleotide primers specific for ABC1 (0.25 μM) below:

| | | |
|---|---|---|
| forward primer: | 5′-CTACCCACCCTATGAACAAC-3′ | (nt 75-94 of ABC1 cDNA); |
| backward primer: | 5′-1GCCACCCCGTATGAACAGGG-3′ | (nt 6731-6751 of ABC1 cDNA). |

These oligonucleotide primers were synthesized by the phosphoramidite method on a DNA synthesizer of the ABI 394 type (Applied Biosystems, Foster City, Calif., USA).

The sites recognized by the restriction enzyme NotI were incorporated into the amplified cDNA of 6676 bp by a new amplification step using 50 ng of human ABC1 cDNA as template, and 0.25 μM of the oligonucleotide primers described above containing, at their 5′ end, the site recognized by the restriction enzyme NotI, in the presence of 200 μM of each of said dideoxynucleotides dATP, dCTP, dTTP and dGTP as well as the Pyrococcus furiosus DNA polymerase (Stratagene, Inc. La Jolla, Calif., USA).

The PCR reaction was carried out over 30 cycles each comprising a step of denaturation at 95° C. for one minute, a step of renaturation at 50° C. for one minute and a step of extension at 72° C. for two minutes, in a thermocycler apparatus for PCR (Cetus Perkin Elmer Norwalk, Conn., USA).

II. Cloning of the cDNA of the Human ABC1 Gene into an Expression Vector:

The 6676 bp insert of the human ABC1 cDNA was cloned into the NotI restriction site of the expression vector pCMV containing a cytomegalovirus early promoter and an enhancer sequence as well as the SV40 polyadenylation signal (Beg et al., 1990; Applebaum-Boden, 1996), in order to produce the expression vector designated pABC1.

The sequence of the cloned cDNA was confirmed by sequencing on the two strands using the reaction set “ABI Prism Big Dye Terminator Cycle Sequencing ready” (marketed by Applied Biosystems, Foster City, Calif., USA) in a capillary sequencer of the ABI 1310 type (Applied Biosystems, Foster City, Calif., USA).

III. Construction of a Recombinant Adenoviral Vector Containing the cDNA of the Human ABC1 Gene

A—Modification of the Expression Vector pCMV-β.

The β-galactosidase cDNA of the expression vector pCMV-β(Clontech, Palo Alto, Calif., USA, Gene Bank Accession No. U02451) was deleted by digestion with the restriction endonuclease NotI and replaced with a multiple cloning site containing, from the 5′ end to the 3′ end, the following sites:

NotI, AscI, RsrII, AvrlI, SwaI, and NotI (sequence of the multiple cloning site:

5′-CGGCCGCGGCGCGCCCGGACCGCCTAGGATTTAAATCGCGGCCCGC
G-3′

this multiple cloning site having been cloned at the level of the NotI site.

The DNA fragment between the EcoRI and SanI sites of the modified expression vector pCMV was isolated and cloned into the modified XbaI site of the shuttle vector pXCXII (McKinnon et al., 1982; McGrory et al., 1988).

B—Modification of the Shuttle Vector PXCXII.

A multiple cloning site comprising, from the 5′ end to the 3′ end the XbaI, EcoRI, SfiI, PmeI, NheI, SrfI, PacI, SalI and XbaI restriction sites (having the sequence:

5′-GCTCTAGAATTCGGCCTCCGTGGCCGTTTAAACGCTAGCGCCCGGG
CTTAATTAAGTCGACTCTAGAGC-3′)

was inserted at the level of the XbaI site (nucleotide at position 3329) of the vector pXCXII (McKinnon et al., 1982; McGrory et al., 1988).

The EcoRI-SalI DNA fragment isolated from the modified vector pCMV-β containing the CMV promoter/enhancer, the donor and acceptor splicing sites of FV40 and the polyadenylation signal of FV40 was then cloned into the EcoRi-SalI site of the modified shuttle vector pXCX, designated pCMV-11.

C—Preparation of the Shuttle Vector pAD12-ABC1.

The human ABC1 cDNA is obtained by an RT-PCR reaction, as described above, and cloned at the level of the NotI site into the vector pCMV-12, resulting in the obtaining of the vector pCMV-ABC1.

The ABC1 cDNA contained in the vector pCMV-ABC1 consists of a DNA fragment of 6676 bp comprising the sequence going from the nucleotide at position 75 to the nucleotide at position 6751 of the human ABC1 cDNA.

D. Construction of the ABC1 Recombinant Adenovirus.

The ABC1-rldV recombinant adenovirus containing the human ABC1 cDNA was constructed according to the technique described by McGrory et al. (1988).

Briefly, the vector pAD12-ABC1 was cotransfected with the vector tGM17 according to the technique of CHEN and OKAYAMA (1987).

Likewise, the vector pAD12-Luciferase was constructed and cotransfected with the vector pJM17.

The recombinant adenoviruses were identified by PCR amplification and subjected to two purification cycles before a large-scale amplification in the human embryonic kidney cell line HEK 293 (American Type Culture Collection, Rockville, Md., USA).

The infected cells were collected 48 to 72 hours after their infection with the adenoviral vectors and subjected to five freeze-thaw lysing cycles.

The crude lysates were extracted with the aid of Freon (Halocarbone 113, Matheson Product, Scaucus, N.J. USA), sedimented twice in cesium chloride supplemented with 0.2% murine albumine (Sigma Chemical Co., St Louis, Mo., USA) and dialysed extensively against buffer composed of 150 nM NaCl, 10 mM Hepes (pH 7,4), 5 mM KCl, 1 mM MgCl₂, and 1 mM CaCl₂.

The recombinant adenoviruses were stored at −70° C. and titrated before their administration to animals or their incubation with cells in culture.

The absence of wild-type contaminating adenovirus was confirmed by screening with the aid of PCR amplification using oligonucleotide primers located in the structural portion of the deleted region.

IV Validation of the Expression of the Human ABC1 cDNA

Polyclonal antibodies specific for the human ABC1 polypeptide were prepared in rabbits and chicks by injecting the synthetic polypeptide “LHKNQTWDVAVLTSFLQDEKVKESYV”, derived from the ABC1 protein. These polyclonal antibodies are used to detect and/or quantify the expression of the human ABC1 gene in cells and animal models by immunoblotting and/or immunodetection.

The biological activity of ABC1 may be monitored by quantifying the cholesterol fluxes induced by apoA-I using cells transfected with the vector pCMV-ABCI which have been loaded with cholesterol (Remaley et al., 1997).

V. Expression In Vitro of the Human abc1 cDNA in Cells.

Cells of the HEK293 line and of the COS-7 line (American Tissue Culture Collection, Betesda, Md., USA), as well as fibroblasts in primary culture derived from Tangier patients or from patients suffering from hypo-alphalipoproteinemia are transfected with the expression vector pCMV-ABC1 (5-25 μg) using Lipofectamine (BRL, Gaithersburg, Md., USA) or by coprecipitation with the aid of calcium chloride (Chen et al., 1987).

These cells may also be infected with the vector pABC1-AdV (Index of infection, MOI=10).

The expression of human ABC1 may be monitored by immunoblotting as well as by quantification of the efflux-of cholesterol induced by apoA-1 using transfected and/or infected cells.

The complementation of the genetic defect from which the Tangier patients and the hypo-alphalipoproteinemic patients are suffering using fibroblasts of these patients, may be confirmed by the detection of the expression of the normal ABC1 gene, which makes it possible to establish the functional importance of this receptor.

VI. Expression In Vivo of the ABC1 Gene in Various Animal Models.

An appropriate volume (100 to 300 μl) of a medium containing the purified recombinant adenovirus (pABC1-AdV or pLucif-AdV) containing from 10⁸ to 10⁹ lysis plaque-forming units (PFUs) are infused into the Saphenous vein of mice (C57BL/6, both control mice and models of transgenic or knock-out mice) on day 0 of the experiment.

The evaluation of the physiological role of the ABC1 protein in the metabolism of lipoprote ins is carried out by determining the total quantity of cholesterol, of triglycerides, of phospholipids and of free cholesterol (Sigma and Wako Chemicals, Richmond, Va., USA), of cholesterol-HDL (CIBA-Corning, Oberlin, Ohio, USA) and apolipoproteins A-I, A-II, E and B from mice (Foger et al., 1997), before (day zero) and after (days 2, 4, 7, 10, 14) the administration of the adenovirus.

Kinetic studies with the aid of radioactively labelled produces such: as apoA-I-HDL, CE-HDL as well as apoB-LDL and CE-LDL are carried out on day 5 after the administration of the vectors rLucif-AdV and rABC1-AdV in order to evaluate the effect of the expression of ABC1 on the metabolism of the HDLs and of the LDLs as well as on the release of cholesterol toward the liver.

The effect of the expression of ABC1 on the development of atherosclerosis may be evaluated by quantifying the mean surface area of aortic lesion in apoE mice after administration of the vector rABC1-Adv.

Furthermore, transgenic mice and rabbits overexpressing the ABC1 gene may be produced, in accordance with the teaching of Waisman (1995) and Hoeg (1996) using constructs containing the human ASC1 cDNA under the control of endogenous promoters such as ABC1, CMV or apoE.

The evaluation of the long-term effect of the expression of ABC1 on the kinetics of the plasma lipids, lipoproteins and apolipoproteins and on atherosclerosis may be carried out as described above.

### Example 17

### Use of Vesicles for the Screening of Agonist and Antagonist Molecules for the ABC1 Protein

The basis of this test is the reconstitution of membranes which have incorporated the ABC1 protein and containing substrates such as cholesterol or phopholipids. The ABC1 protein may then be activated or its function repressed by the addition of molecules of interest. The outflow of the substrates through the channel formed by the ABC1 protein is then detected.

a) Reconstitution of a Membrane Containing the ABC1 Protein and a Labelled Lipid Substrate.

Various strategies may be used to manufacture these membranes, methods using organic solvents, mechanical means such as sonication, the “French press”, or by freeze-thaw cycles or using detergents (cholates, Chaps, Chapso) (reference: Rigaud et al. Biochimica et Biophysica Acta 1231 (1995) 223-246). More particularly, a lipid substrate such as phospholipids, cholesteol or cholesterol ester, a radioactive substrate of the 3H-cholesterol, 125-I-cholesterol or 3H-phospphatidylcholine type or a fluorescent substrate with NBD or pyrene (Molecular Probes; http://www.probes.com) and phospatidylecholine from eggs (1 mM) are dried on the pellet of a glass flask. Sodium cholate and the ABC1 protein are mixed in this flask in a mol to mol ratio of 0.3. The whole is vortex-mixed for 5 minutes and then incubated at 25° C. for 30 minutes and then dialysed against a saline buffer. The proteoliposome produced according to this protocol is monitored by turbidimetry in order to verify that its manufacture is good.

b) Capture of the Proteoliposome on a Solid Surface

This step may be carried out by incorporating binding proteins of the integrine type. In this protocol, a capture by the antibodies directed against the ABC1 protein and previous adsorbed on a 96- or 384-well plate is used.

A solution containing these antibodies at the concentration of 100 μg/l are absorbed on these multiwell plates by incubating overnight at 4° C. After washing, the plate is then saturated with bovine albumine at 1 mg/ml incubated for 2 hours at 37° C. The whole is then washed and incubated with the proteoliposomes containing ABC1 for 2 hours at 37° C.

c) Binding to the Molecules of Interest

This step is carried out by incubation of products for 1 hour at 37° C.

d) Determination of the Activation or Inhibition of the ABC1 Protein

If the substrate if fluorescent, the fluorescence of the supernatant shows us the activity of a product in inducing a transport of lipid to the outside of the proteoliposome. Alternatively, the use of a Confocal system gives us information on the quantities of substrate inside and outside the proteoliposome. If the substrate is radioactive, the use of CytoStar-type plates having a bottom with scintillation liquid makes it possible to reveal the substrate still sequestered in the proteoliposome.

### Example 18

### Use of Anion Transport for the Screening of Agonist et Antagonist Molecules for the ABC1 Protein).

The principle of this test lies in the property which the A8C1 protein has for transporting the anions during its activation.

a) The macrophage cells of the THP-1 lines, monocytic leukemia human cells, are a model of differentiated macrophages. The cells are cultured in an RPMI 1640 medium supplemented with 10% foetal calf serum in 48-multiwell plates at the density of 2 105 cells per well. The fibroblast cells of patients suffering from Tangier disease may be used as negative control because their ABC1 protein is not functional. Another negative control may be obtained by the addition of anti-ABC1 antibodies.

b) The use of anion transport defective cells or cells treated with anion channel inhibitors (Verapamil type, an inhibitor of P-glycoprotein or tetraethylammonium, a potassium channel inhibitor) may also be used.

c) For the actual test itself, the cells are then washed with an Earles's modified salt solution (ESS) medium preloaded with 1 ml of KI at 1 μmol/L (0.1 μCi/ml of NaI125) in this ESS medium for 30 minutes at 37° C. The products are then added to the extracellular medium. The cells are then washed with the ESS medium.

d) The quantity of iiodide in the medium is detected every minute for 11 minutes. The first two points correspond to the basal efflux. At the end of the incubation, the medium is taken up and the quantity of iodine remaining in the cells is counted following lysis of the cells in 1 molar NaOH.

e) The total quantity of radioactivity at time zero is equal to the sum of the radioactivity found in the supernatant and the residual radioactivity in the cells. The efflux curves are constructed by plotting the percentage of radioactivity released into the medium as a function of time.

### Example 19

### Use of THP-1 Macrophages Expressing IL-1Beta for the Screening of Agonist and Antagonist Molecules for the ABC1 Protein)

The principle of this test is that any substances modulating the activity of the ABC1 protein has repercussions on the synthesis of IL-1 beta.

a) The macrophage cells of the THP-1 lines, monocytic leukemia human cells, are a model of differentiated macrophages. The cells are cultured in an RPMI 1640 medium supplemented with 10% foetal calf serum in multiwell plates at the density of 2 105 cells per well.

b) For the actual test itself, the cells are then washed and placed in an RPMI 1640 medium containing 1 mg/ml of purified human albumine fraction IV.

c) The products are added to the extracellular medium. Simultaneously, the cells are then activated by addition of lipopolysaccharide (LPS) over 3 hours at 1 μg/ml followed by an incubation of 30 minutes in the presence of ATP at 5 mmol/L.

d) The concentrations of IL-1beta and of control IL-1alpha, tumor necrosis factor alpha (TNFalpha) and IL-6 are determined by ELISA kits according to the manufacturers' instructions (R&D Sytem; human IL-1beta Chemiluminescent ELISA reference QLB00). The variations of mRNA for IL-1beta which is not supposed to be affected are evaluated by the Nothern blotting technique with the corresponding probe.

### REFERENCES

Altschul S. F. et al, J. Mol. Biol. 1990 215 403-410

Altschul S. F. et al, Nucleic Acids Res. 1997 25: 3389-3402

Applebaum-Boden, JCI 97, 1996

Ausubel et al., 1989, Current Protocols in Molecular Biology, Green Publishing Associates and Wiley interscience, N.Y

Beard et al., Virology 75 (1990) 81

Beaucage et al., Tetrahedron Lett 1981, 22: 1859-1862

Becq et al Journal of Biological Chemistry vol 272, No. 5 pages 2695-2699, 1997

Beg et al PNAS 87 p3473 1990

Bender et al., J. Virol. 61 (1987) 1639

Bernstein et al. Genet. Eng. 7 (1985) 235

Boulianne et al; Nature 312:643 (1984)

Breakfield et al., New Biologist 3 (1991) 203

Brown E L, Belagaje R, Ryan M J, Khorana H G, Methods Enzymol 1979;68:109-151

Cabilly et al., U.S. Pat. No. 4,816,567

Chen and Kwok Nucleic Acids Research 25:347-353 1997

Chen et al. Proc. Natl. Acad. Sci. USA 94/20 10756-10761,1997

Chen et al., 1987, Mol. Cell. Biol., 7: 2745-2752.

Chomczynski, P. and Sacchi, 1987, Anal. Biochem., 162, 156-159.

DeRisi J. et al., 1997, Science, 278, 680-686.

Dib C. et al, 1996, Nature, 380: 152-154.

Felgner et al., PNAS 84 (1987) 7413

Flotte et al, 1992, Am. J. Respir. Cell Mol. Biol., 7: 349-356.

Forsell Y. et al., 1997, Biol. Psychiatry, 42: 898-903

Fraley et al., 1979, Proc. Natl. Acad. Sci. USA, 76: 3348-3352.

Fraley et al., J. Biol. Chem. 255 (1980) 10431

Francis et al., 1995, J. Clin. Invest., 1:78-87

Fromont-Racine et al, 1993. Nucleic Acid Res. 21(7):1683-1684

Fuller S. A. et al., 1996, Immunology in Current Protocols in Molecular Biology, Ausubel et al.

Gopal, 1985, Mol. Cell. Biol., 5: 1188-1190.

Graham et al., 1973, Virology, 52: 456-457.

Graham et al., J. Gen. Virol. 36 (1977) 59

Haff L. A. and Smirnov I. P., Genome Research, 7:378-388, 1997

Harn, Methods Cell. Biol. 21a (1980) 255

Hames B D and Higgins S J, 1985, “Nucleic acid hybridization: a practical approach”, Hames and Higgins Ed., IRL Press, Oxford.

Hammeling et al., in: Monoclonal Antibodies and T-Cell Hybridomas, Elsevier, N.Y., pp. 563-681 51981

Harland et al., 1985, J. Cell. Biol., 101: 1094-1095.

Hoeg PNAS 93 p11448, 1996

Horvath S. et al., 1998, Am. J. Hum. Genet., 1998, 63: 1886-1897.

Houben Weyl, 1974, in Meuthode der Organischen Chemie, E. Wunsch Ed., Volume 15-I and 15-II,

Huygen et al., 1996, Nature Medicine, 2(8):893-898

Kaneda et al., Science 243 (1989) 375

Kim et al. Genomics (1996), 34:213-218)

Koch Y., 1977, Biochem. Biophys. Res. Commun., 74:488-491

Köhler et al, Eur. J. Immunol. 6:511 (1976)

Köhler et al, Eur. J. Immunol. 6:292 (1976)

Köhler et al, Nature 256:495 (1975)

Kohler G. and Milstein C., 1975, Nature, 256: 495.

Kozbor et al., 1983, Hybridoma, 2(1):7-16.

Lander and Schork, Science, 265, 2037-2048, 1994

Langmann T. et al., 1999, Biochem. Biophys. Res. Comm., 257: 29-33.

Leger O J, et al., 1997, Hum Antibodies, 8(1): 3-16

Levrero et al., Gene 101 (1991

Luciani M. F. et al., 1994, Genomics, 21: 150-159.

Lyer V. et al., 1999, Science, 283: 83-87.

MacKinnon et al Gene 19 p33 1982

Maniatis et al. Molecular cloning: A Laboratory Manual, Cold Spring Harbor Press, Cold Spring, N.Y. 1982

Marcil M. et al., 1999, Artreriosclerosis Throbosis and Vascular Biology, 17 1813-1821.

Martineau P, Jones P, Winter G, 1998, J Mol Biol, 280(1):117-127

McCormick, BioTechnology 3 (1985) 689

McGrory et al (Virology, 163, 614,1988

McLaughlin B A et al., 1996, Am. J. Hum. Genet., 59: 561-569.

Merrifield R B, 1965a, Nature, 207(996): 522-523.

Merrifield R B., 1965b, Science, 150(693): 178-185.

Morrison et al., EP 173494

Morrison, Science 229:1202 (1985)

Narang S A, Hsiung H M, Brousseau R, Methods Enzymol 1979;68:90-98

Neuberger et al., Nature 314: 268 (1985)

Neuberger et al., WO 8601533

Nickerson D. A. et al., Genomics, 1992, 12: 377-387.

Nicolau C. et al., 1987, Methods Enzymol., 149:157-76.

Oi et al., Biotechnique 4:214 (1986)

Okayama (Mol Cell. Biol. 7: 2745, 1987

Pagano et al., J. Virol. 1 (1967) 891

Potter et al., 1984, Proc Natl Acad Sci USA.; 81(22):7161-5

Reimann K A, et al., 1997, AIDS Res Hum Retroviruses. 13(11): 933-943

Remaley et al. ATVB 17,1813,1997 Chen et at Mol Cell Biol 7 p2745 1987

Ridder R, Schmitz R, Legay F, Gram H, 1995, Biotechnology (N Y), 13(3):255-260

Rigaud et al. Biochimica et Biophysica Acta 1231 (1995) 223-246

Robinson et al., WO 8702671

Rust S. et al., Nature Genetics, vol. 20, September 1998, pages 96-98

Sambrook, J. Fritsch, E. F., and T. Maniatis. 1989. Molecular cloning: a laboratory manual. 2ed. Cold Spring Harbor Laboratory, Cold spring Harbor, N.Y.

Samulski et al., 1989, J. Virol., 63: 3822-3828.

Sanchez-Pescador R., 1988, J. Clin. Microbiol., 26(10):1934-1938

Sham P. C. et al., Ann. Hum. Genet., 1995, 59: 323-336.

Sternberg N. L., 1992, Trends Genet., 8: 1-16.

Sternberg N. L., 1994, Mamm. Genome, 5: 397404.

Strautnieks S. S. et al., 1998, Nature Genetics, 20: 233-235.

Tacson et al., 1996, Nature Medicine, 2(8):888-892.

Taniguchi et al., EP 171496

Tur-Kaspa et al, 1986, Mol. Cell. Biol., 6: 716-718.

Urdea M. S., 1988, Nucleic Acids Research, 11: 49374957

Urdea M S et al., 1991, Nucleic Acids Symp Ser., 24: 197-200.

Vaisman J B C 270 p12269, 1995

Van, den Hazel. H., H. Pichler, V. M. M. do, E. Leitner, A. Goffeau, and G.

Daum. 1999. PDR16 and PDR17, two homologous genes of Saccharomyces cerevisiae, affect lipid biosynthesis and resistance to multiple drugs. J Biol Chem 274 (4):1934-41.

Wands et al., Gastroentérology 80:225-232 (1981)

Xiong M. et al., 1999, Am. J. Hum. Genet., 64: 629-640.

Yamon et al Blood vol 90, No. 8 pages 2911-2915, 1997

1. Nucleic acid comprising at least 245 consecutive nucleotides of a polynucleotide chosen from the group consisting of the nucleotide sequences SEQ ID NO 1-14, or a nucleic acid having a complementary sequence.

2. Nucleic acid comprising a polynucleotide chosen from the group consisting of the nucleotide sequences SEQ ID NO 15-47, or a nucleic acid having a complementary sequence.

3. Nucleic acid comprising at least 8 consecutive nucleotides of a polynucleotide chosen from the group consisting of the nucleotide sequences SEQ ID NO 48-90, or a nucleic acid having a complementary sequence.

4. Nucleic acid having at least 80% nucleotide identity with a polynucleotide chosen from the group consisting of the nucleotide sequences SEQ ID NO 48-90, or a nucleic acid having a complementary sequence.

5. Nucleic acid hybridizing, under high stringency conditions, with a polynucleotide chosen from the group consisting of the nucleotide sequences SEQ ID NO 48-90, or a nucleic acid having a complementary sequence.

6. Nucleic acid comprising a polynucleotide having the sequence SEQ ID NO 91, or a nucleic acid having a complementary sequence.

7. Nucleic acid comprising at least eight consecutive nucleotides of a polynucleotide chosen from the group consisting of the nucleotide sequences SEQ ID NO 92, a biologically active fragment thereof or a nucleic acid having a complementary sequence.

8. Nucleic acid having at least 80% nucleotide identity with a polynucleotide chosen from the group consisting of the nucleotide sequences SEQ ID NO 92, a biologically active fragment thereof or a nucleic acid having a complementary sequence.

9. Nucleic acid hybridizing, under high stringency conditions, with a polynucleotide chosen from the group consisting of the nucleotide sequences SEQ ID NO 92, a biologically active fragment thereof or a nucleic acid having a complementary sequence.

10. Nucleic acid having at least eight consecutive nucleotides of a polynucleotide chosen from the group consisting of the nucleotide sequences SEQ ID NO 93-96, or a nucleic acid having a complementary sequence.

11. Nucleic acid having at least 80% nucleotide identity with a polynucleotide chosen from the group consisting of the nucleotide sequences SEQ ID NO 93-96, or a nucleic acid having a complementary sequence.

12. Nucleic acid hybridizing, under high stringency conditions, with a polynucleotide chosen from the group consisting of the nucleotide sequences SEQ ID NO 93-96, or a nucleic acid having a complementary sequence.

13. Nucleic acid having at least eight consecutive nucleotides of a polynucleotide chosen from the group consisting of the nucleotide sequences SEQ ID NO 97-108 and comprising the polymorphic base, or a nucleic acid having a complementary sequence.

14. Nucleotide probe or primer specific for the ABC1 gene, having a length of at least 15 nucleotides, chosen from the nucleic acids according to any one of claims 1 to 9.

15. Probe or primer according to claim 14, comprising a polynucleotide chosen from the group consisting of the nucleotide sequences SEQ ID NO 109-138, or a nucleic acid having a complementary sequence.

16. Nucleotide probe or primer useful for the detection of a mutation in the ABC1 gene, having a length of at least 15 nucleotides, chosen from the nucleic acids according to either of claims 11 and 12.

17. Nucleotide probe or primer according to claim 16, comprising a polynucleotide chosen from the nucleotide sequences SEQ ID NO 109-112, or a nucleic acid having a complementary sequence.

18. Nucleotide probe or primer useful for the detection of a polymorphism in the ABC1 gene, having a length of at least 15 nucleotides, chosen from the nucleic acids according to claim 13.

19. Probe or primer according to claim 18, comprising a polynucleotide chosen from the nucleotide sequences SEQ ID NO 142-149, or a nucleic acid having a complementary sequence.

20. Nucleotide primer comprising at least 15 consecutive nucleotides of a polynucleotide chosen from the group consisting of the nucleotide sequences SEQ ID NO 97-108 or of their complementary sequences, the base of the 3′ end of these primers being complementary to the nucleotide located immediately on the 5′ side of the polymorphic base of one of the sequences SEQ ID NO 97-108 or of their complementary sequences.

21. Nucleotide primer comprising at least 15 consecutive nucleotides of a polynucleotide chosen from the group consisting of the nucleotide sequences SEQ ID NO 97-108 or of their complementary sequences, the base of the 3′ end of these primers being complementary to a nucleotide situated at 2, 3, 4, 5, 6, 7, 8, 9, 10, 11, 12, 13, 14, 15, 16, 17, 18, 19, 20 nucleotides or more on the 5′ side of the polymorphic base of one of the sequences SEQ ID NO 97-108 or of their complementary sequences.

22. Method of amplifying a nucleic acid according to any one of claims 1 to 13 contained in a sample, said method comprising the steps of:
a) bringing the sample in which the presence of the target nucleic acid is suspected into contact with a pair of nucleotide primers whose hybridization position is located respectively on the 5′ side and on the 3′ side of the region of the target nucleic acid whose amplification is sought, in the presence of the reagents necessary for the amplification reaction; and
b) detecting the amplified nucleic acids.

23. Method of amplification according to claim 22, characterized in that the nucleotide primers are chosen from the primers according to any one of claims 14 to 19.

24. Box for amplifying a nucleic acid according to any one of claims 1 to 13 comprising:
a) a pair of nucleotide primers whose hybridization position is located respectively on the 5′ side and 3′ side of the target nucleic acid whose amplification is sought;
b) where appropriate, the reagents necessary for the amplification reaction.

25. Box for amplifying a nucleic acid according to claim 22, characterized in that the nucleotide primers are chosen from the group consisting of the primers according to any one of claims 14 to 19.

26. Nucleotide probe according to any one of claims 14 to 19, characterized in that it comprises a marker compound whose presence is detectable.

27. Method of detecting the presence of a nucleic acid according to any one of claims 1 to 13 in a sample, said method comprising the steps of:
a) bringing one or more nucleic probes according to one of claims 14 to 19 into contact with the sample to be tested;
b) detecting the complex which may have formed between the probe(s) and the nucleic acid present in the sample.

28. Method of detection according to claim 27, characterized in that the probe(s) are immobilized on a support.

29. Box for detecting the presence of a nucleic acid according to any one of claims 1 to 13 in a sample, said box comprising:
a) one or more nucleotide probes according to any one of claims 14 to 19;
b) where appropriate, the reagents necessary for the hybridization reaction.

30. Box for detection according to claim 29, characterized in that the probe(s) are immobilized on a suppport.

31. Recombinant vector comprising a nucleic acid according to one of claims 1 to 13.

32. Vector according to claim 31, characterized in that it is an adenovirus.

33. Vector according to either of claims 32 and 33, characterized in that it is ABC1-rldV

34. Recombinant host cell comprising a nucleic acid according to one of claims 1 to 13 or a recombinant vector according to one of claims 31 to 33.

35. Mutated ABC1 polypeptide, characterized in that it comprises a polypeptide having the amino acid sequence SEQ ID NO 140.

36. Mutated ABC1 polypeptide, characterized in that it comprises a polypeptide having the amino acid sequence SEQ ID NO 141.

37. Antibody directed against a mutated ABC1 polypeptide according to either of claims 35 and 36, or a peptide fragment thereof.

38. Antibody according to claim 37, characterized in that it comprises a detectable compound.

39. Method of detecting the presence of a polypeptide according to either of claims 35 and 36 in a sample, comprising the steps of:
a) bringing the sample into contact with an antibody according to either of claims 37 and 38;
b) detecting the antigen/antibody complex formed.

40. Diagnostic box for detecting the presence of a polypeptide according to either of claims 35 and 36 in a sample, said box comprising:
a) an antibody according to either of claims 37 and 38;
b) a reagent allowing the detection of the antigen/antibody complexes formed.

41. Pharmaceutical composition intended for the prevention of or treatment of subjects affected by, a dysfunction in the reverse transport of cholesterol, comprising a nucleic acid according to either of claims 1 and 6, in combination with one or more physiologically compatible excipients.

42. Pharmaceutical composition intended for the prevention of or treatment of subjects affected by, a dysfunction in the reverse transport of cholesterol, comprising a recombinant vector according to claim 31, in combination with one or more physiologically compatible excipients.

43. Use of a nucleic acid according to one of claims 1 and 6 for the manufacture of a medicament intended for the prevention of Atherosclerosis in various forms or more particularly for the treatment of subjects affected by a dysfunction in the reverse transport of cholesterol.

44. Use of a recombinant vector according to claim 31 for the manufacture of a medicament intended for the prevention of Atherosclerosis in various forms or more particularly for the treatment of subjects affected by a dysfunction in the reverse transport of cholesterol.

45. Use according to claim 44, characterized in that the vector is ABC1-rldV.

46. Use of the ABC1 polypeptide having the sequence SEQ ID NO 139 for the manufacture of a medicament intended for the prevention of Atherosclerosis in various forms or more particularly for the treatment of subjects affected by a dysfunction in the reverse transport of cholesterol.

47. Pharmaceutical composition for the prevention of or treatment of subjects affected by, a dysfunction in the reverse transport of cholesterol, comprising a therapeutically effective quantity of the polypeptide having the sequence SEQ ID NO 139.

48. Use of the ABC1 polypeptide, or of cells expressing the ABC1 polypeptide, for screening active ingredients for the prevention or treatment of diseases resulting from a dysfunction in the reverse transport of cholesterol.

49. Method of screening a compound active on the metabolism of cholesterol, an agonist or antagonist of the ABC1 polypeptide, said method comprising the following steps:
a) preparing membrane vesicles containing the ABC1 polypeptide and a lipid substrate comprising a detectable marker;
b) incubating the vesicles obtained in step a) with an agonist or antagonist candidate compound;
c) qualitatively and/or quantitatively measuring the release of the lipid substrate comprising a detectable marker;
d) comparing the measurement obtained in step b) with a measurement of the release of the labeled lipid substrate by vesicles which have not been previously incubated with the agonist or antagonist candidate compound.

50. Method of screening a compound active on the metabolism of cholesterol, an agonist or antagonist of the ABC1 polypeptide, said method comprising the following steps:
a) obtaining cells, for example a cell line, expressing naturally or after transfection the ABC1 polypeptide;
b) incubating the cells of step a) in the presence of an anion labeled with a detectable marker;
c) washing the cells of step b) in order to remove the excess of the labeled anion which has not penetrated into these cells;
d) incubating the cells obtained in step c) with an agonist or antagonist candidate compound for the ABC1 polypeptide;
e) measuring the efflux of the labeled anion;
f) comparing the value of the efflux of the labeled anion determined in step e) with the value of the efflux of the labeled anion measured with cells which have not been previously incubated in the presence of the agonist or antagonist candidate compound for the ABC1 polypeptide.

51. Method of screening a compound active on the metabolism of cholesterol, an agonist or antagonist of the ABC1 polypeptide, said method comprising the following steps:
a) culturing cells of a human monocytic line in an appropriate culture medium, in the presence of purified human albumin;
b) incubating the cells of step a) simultaneously in the presence of a compound stimulating the production of IL-1 beta and of the agonist or antagonist candidate compound;
c) incubating the cells obtained in step b) in the presence of an appropriate concentration of ATP;
d) measuring IL-1 beta released into the cell culture supernatant.
e) comparing the value of the release of the IL-1 beta obtained in step d) with the value of the IL-1 beta released into the culture supernatant of cells which have not been previously incubated in the presence of the agonist or antagonist candidate compound.


SEQUENCE LISTING (159 sequences)

SEQ ID NO: 1 - DNA, 3153 bp, Homo sapiens
acatggcaatggcattcattaggaatctagctgggaaaatccagtgtgtatgcttggaaatgagggatctggggctggagagaaaggcatgggcatgccttggagggacttgtgtgtcaagctgaggacctttactttaagctctaggggaccaggcaaggggagatgtagatacgttactctgatggggtggatgaattgaagaaggatgaggcaagaatgaaggcagagaccagggaggaggctctccaagtggccaaggcataaagcaagaaatgaggcctggtgactgcttagtggcagagcagtgaaagagagggaggcatcaaagtgagtctcgatttctagctgggtgggtggtagcgatgtccagtaggccagtggctactgaggtctgcagtggaggagggtggttgggctggagacagatgatgagggagtcatcagcctgtgggtggaagaaaagggaacctcttccaactgttttctttgcttcttccctctctttctctttttttttttttttggacagagtcttgctctgtcacccaggctgaaatgcagtggcatgatcttggctcaccacagcctccgcctcctgggttcaagcaattctcctgtctcagcctccagagtagctgggattacaggcacatatcactgtgcccggctaatttttgtattttcagtggagatgggatttcaccatgttggtcgggctggaatgaactcctgacctcaagtgatccacctgcctcagcctcccaaagtgttgggattacaggcattgagccaccgcgcccggcctttcttccctctcttaaagagtgtttatttaattccacaaacatgagcttgtcaccccctgtagcctggcatctcctacacgaggtgatggctgaggcttctgcttctgctggggtagctctgatctttctgctttctctggcactgtctacccatgttgcctcaccccacaggtcccagggcacctctctcgggcaagtcttggaaccctctgacactgatttgctctcttttctgagctgcttttagccacccatcctcgggacctgttttctctctgcctccacccctgcgggcagtcttaggtctcctgcccctcacgagcaccccagagaggccacgtgctcagtgatctcagtgggcgcatctttctagtcttgctattctttttggccatgttgttcagaaaccatactgggcagggccgacttcaccctaaaggctgcgtctcttcactctgcttttgtttgttccaaataaagtggcttcagaattgctaaccctagcctctgtgaacttgtgaggtacaattttgtgtctgttatgttaacaaaaatacatacataccttcctggtgatggtataaattgctattctctattggaaagcaatttggaatgaaaatttaaagaaccattttaaaatatgctatcctgcgtacctccattccacccacccccagggatgtagcctactgaaataattttaaagaagtcaccatatgagagaaaatgttattgctatattgttattgtgagaaattggaaatagactaaatgttcagcactataggaataattaatgaaattacatatactctatacaatcattatgctgccattgaaataataaatacaaaggcgcaaggggggaaaagcttataatgttagtgaaactaagactgatttttttataaagcagcagttttcagacccttggagactccaattcggtagaaccagagcttcatcttctctgtcgaagctgtgacaggagttgcaaatgcctctcctttttgctgagtttgcagctgctgtttttccggcagcacatctgtgcaggcctctgcctcggcccctctggatctgctgattgagcagcggattgatctgtccttctctttcgtgttgacccatgtgaggaaccaactggcaagggaacaagaaatggaaataggcctcctttgcatcatgacctgtacatcctgcaattggaaaagattgtactttagttggtttaaccagcagcattatttttctaaactaagcagtaagaaggaattaggttttatgtgggatcaacagactgggtctcaaaagaggaaggtgatagaacacagtggggagggggaggtgcactagaaacagagggcctatgctttcattctggctttgctacttaatagctgtgtgacccaatcttagagacttaacctctctgaacttccattttctcatgtataaaatgggaaatattaaaggatactcactgggctggtggcttgtgcctgtaatcccagcacttggggaggttgaggtgggaggatcacttgagcccaggtgttcaagaccagcccaggcaacatggcaagactctgtctctatgaaaaaattaaaaattagccaggtgtggtggtgtgcacctgtagtcttagctacttggtaggctgagatgggaggatcacttgggcttgggaggtcaaggctgcggtgagctgtgattccatcactgcactccagcccgggcggcagagcgagacactgaatccaaacgacaacaacaacaaaaggcaaaaaaataaaagtgccctctttatggagttgtgtaaggtgaagcatatacactattcaacatagtaactatataaaggaagtattgttgttgttactgtagttaataccattaagtgagatgtttcgtatagtggaaagcacatggactctgaattcagactggtctgactttgagtctcagctccacatctagtaatactatgaccaagccctggttaaaatcatgtttttttttcttcagccttagtcttctcacatataaaatagggacactgtcatttacctcagttttctgtgaggataaaacaacgacagtgtatatgcaagtattttgtaaattttgtagtgctcctcaagatttagttggtgtttactacttgtactttctcactggaatggcagatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctt

SEQ ID NO: 2 - DNA, 7660 bp, Homo sapiens
  misc_feature (3854)..(3854): n is a, c, g, or t
aattcgggtccaattaaatttttgaaattttatattaaaaattatattagtagggatgggtaagaggtgttttggtctggttggttggttagttgctatgactcagaattgctaagaaaacagaaaagtaagataagatcattgttttaacctcttttcctccacaaaatcaataaataacatatccctaaattactcttagaatttctcttaaattgcagtgaaaaaccaaaatccttcattcttggttgaaggttggaaaactacgttagagaggattagagagagaggatgagcaatcgtgtagtcagcccttgcctcctagtgtaggatttgtctcagccactgcttgttgtcctggctgccaacgttctcatgaaggctgttcttctatcagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtaagtggaatcccatcacaccagcctggtcttggggaggtccagagcacctattatattaggacaagaggtactttattttaactaaaaatttggtagaaatttcaacaacaacaaaaaaactcaacttggtgtcatgattttggtgaaattggtacatgacttgctggaaggtttttcataggtcataaaataacagtatcttttgatttagcatttctactcaagggaattaattccaggaattttggtggcaggcacctgtaatcccagctactcgggaggctgaggcaggagaattgcttgaacccaggaggcagaggttgcagtgagctaagatcgcatcattgcactcccgcctgggcaataagagtgaaactccatctcaaaaaaaaaaaaagatacaaaaatagaaaaaggggcttggtaagggtagtagggttttgggcaatttttttttttttttttttttattgtatggttctaaaggaatggttgattacctgtggtttggttttaggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatgtaagttacctgcaagccactgtttttaaccagtttatactgtgccagatgggggtgtatatatgtgtgtgcatgtgcatgcatgtgtgaatgatctggaaataagatgccagatgtaagttgtcaacagttgcagccacatgacagacatagatatatgtgcacacactagtaaacctctttccttctcatccatggttgccacttttatctttttatttttatttttttttttgagatggagtctcgctctgacgcccaggctggagtgcagtggctcgatctcggctcactgcaacctttgcctcccgggttcaagctattctcctgcctcagcctccacagtagctgggactacaggctcatgctgccacgcccggctgactttttgtattttagtagagacgaggtttcaccatgttacccaggctagacttcaactcctgagctcaggcaatccaccctccttggcctcccaaagtgctgggattacaggtgtgagccactgcacccagcccaccactttaattttttacactctacccttttggtcaaaatttgctcaatctgcaagcttaaaatgtgtcatgacaaacacatgcaagcacatactcacacatagatgcagaaacagcgtctaaacttataaaagcacagtttatgtaaatgtgtgcacttcttctccctaggtggtaaaccacatttcaaaacaacccaaataaaactgaacaaagcttcttcctcttagactttttagaaaatctttcagtgctgagtcactaagctgccaagttctcattgtgggaactatgcctttggatgtaatgatttcttctaagacaatgggcggaggtgtagttattgcagacatctgaaatatgtaatgtttcttccagattctggaaattctcttattctctgtggttggtggtggtggtgggatgtgtgtgtgtgtgtgtgtgtgtgtgtgtgtgtgtgtgtagggatcaggatgcgggaggagctgggttctgcttgtattggttctctgttttgcattgaatagtgtgtttccttgtatggctatctatagcttttcaaggtcaccagaaattatcctgtttttcaccttctaaacaattagctggaatttttcaaaggaagacttttacaaagacccctaagctaaggtttactctagaaaggatgtcttaagacagggcacaggagttcagaggcattaagagctggtgcctgttgtcatgtagtgagtatgtgcctacatggtaaagctttgacgtgaacctcaagttcagggtccaaaatctgtgtgcctttttactttgcacatctgcattttctattctagcttggaatctgaaacattgacaagagctgcctgaaatgtatgtctgtggtgtgattagagttacgataagcaagtcaatagtgagatgaccttggagatgttgaacttttgtgagagaatgagttgtttttttgttttggtttttagtactttaacataatctacctttagtttaagtatcgctcacagttacctagttactgaagcaagcccccaaagaaatttggtttggcaacactttgttagcctcgtttttctctctacattgcattgctcgtgaagcattggatcatacgtacatttcagagtctagagggcctgtccttctgtggcccagatgtggtgctccctctagcatgcaggctcagaggccttggcccatcaccctggctcacgtgtgtctttctttctccccttgtccttccttggggcctccagctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaaggtaaggcagcctcactcgctcttccctgccaggaaactccgaaatagctcaacacgggctaagggaggagaagaagaaaaaaaatccaagcctctggtagagaaggggtcatacctgtcatttcctgcaatttcatccatttatagttggggaaagtgaggcccagagaggggcagtgacttgcccaaggtcaacccagccgggtagcagctaagtaggatgagagtgcagggttcatgctttccagataaccacatgctcaactgtgccatgctgtctcattggtagtggttcatggcagcatctgaaagctatttattttcttagatatattgggtggcgattcttcctaagtttctaagaacaataatcagaaggatatatattgttgcaggttagactgtctggaagcagaggctgaaatagagtttgatgtatgggtatttatgagggctcaatacctatgaagagatatggaagatgcaggattgggcagagggaggagttgaactgtgatatagggccaaccccgtggggcactctanagaatatgcagcttgttggagttgttnttcatcgagctgaaacatccagccctttgtgctcccccaaggcctccctcctgacaccacctacctcagccctctcaatcaatcactggatgtgggctgccctgggaaggtcgtgccccagggcctacatggctctctgctgctgtgacaaacccagagttgctgatgcctgaggccgtctactgacagctgggcaacaaggcttccctgaatggggactctgggcagtgcagttttgtgtctgaaccatacattaatatatttatatccgaattttctttctctgcaagcatttcatataaagacacatcaggtaaaaataaatgtttttgaagcaaaaggagtacaaagagataagaactaactaatttaatactagttaccatctgttacaaatagttcctactgattgccaaggactgtttaaacacatcacatgggcttcttcttctatcctcactaacccttttaacagacaaggaaatgaggctcaggaaggtcaaggactttattgaggttccacagtaggatacagttcttgctaaaagcaacccctccctcatgctctgttatctaactgcaaggggaaggtcagtggcagaggtagtggtcccatggttggtgcataagagctgctctgagacaactgcatgctggtgggtcctgcagacatgtacccatcagccggagataggctcaaaatatccacaagagtttggatgattgtgggaatgcagaatccatggtgatcaagagggaaagtcaagttgcctggccattttccttggcttttagacagaaaagttacgtgggatattatctcccacagctcttctgtggtgccaccagtcatagtccttatataaggagaaaccagttgaaattacctattgaagaaacaaagagcaaactcgcccactgaaatgcgtagaaagccctggactctgttgtattcataactctgccattatttttctgcgtagttttgggtaagtcacttatcttctttaggatggtaatgatcagttgcctcatcagaaagatgaacagcattacgcctctgcattgtctctaacatgagtaggaataaaccctgtcttttttctgtagatcatacaagtgagtgcttgggattgttgaggcagcacatttgatgtgtctcttccttcccagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctgtgagtacctctggcctttcttcagtggctgtaggcatttgaccttcctttggagtccctgaataaaagcagcaagttgagaacagaagatgattgtcttttccaatgggacatgaaccttagctctagattctaagctctttaagggtaagggcaagcattgtgttttattaaattgtttacctttagtcttctcagtgaatcctggttgaattgaattgaatggaatttttccgagagccagactgcatcttgaactgggctggggataaatggcattgaggaatggcttcaggcaacagatgccatctctgccctttatctcccagctctgttggctatgttaagctcatgacaaagccaaggccacaaatagaactgaaaactcttgatgtcagagatgacctctcttgtcttccttgtgtccagtatggtgttttgcttgagtaatgttttctgaactaagcacaactgaggagcaggtgcctcatcccacaaattcctgacttggacacttccttccctcgtacagagcagggggatatcttggagagtgtgtgagcccctacaagtgcaagttgtcagatgtccccaggtcacttatcaggaaagctaagagtgactcataggatgctcctgttgcctcagtctgggcttcataggcatcagcagccccaaacaggcacctctgatcctgagccatccttggctgagcagggagcctcagaagactgtgggtatgcgcatgtgtgtgggggaacaggattgctgagccttggggcatctttggaaacataaagttttaaaagttttatgcttcactgtatatgcatttctgaaatgtttgtatataatgagtggttacaaatggaatcattttatatgttacttggtagcccaccactcccctaaagggactctataggtaaatactacttctgcaccttatgattgatccattttgcaaattcaaatttctccaggtataatttacactagaagagatagaaaaatgagactgaccaggaaatggataggtgactttgcctgtttctcacagagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggtacactgctttgggcatctgtttggaaaatatgacttctagctgatgtcctttctttgtgctagaatctctgcagtgcatgggcttccctgggaagtggtttgggctatagatctatagtaaacagatagtccaaggacaggcagctgatgctgaaagtacaattgtcactacttgtacagcacttgtttcttgaaaactgtgtgccaggcagcatgcaaaatgttttatacacattgcttcatttaattctcacaaggctactctgaagtagttactataataaccagcaattttcaaatgagagaactgtgactcaaagacgttaagtaaccagctttggtcacacaactgttaaatgttggtacgtggaggtgaatccacttcggttacactgggtcaataagcccaggcgaatcctcccaatgctcacccaattctgtatttctgtgtcctcagagggggtacaactaggagaggttctgtttcctgagtacaggttgttaataattaaatatactagctctaaggcctgcctgtgatttaattagcattcaataaaaattcatgttgaatttttctttagtacttctttcttaatataatacatcttcttgaccaagtccaagaggaacctgcgttggacagttttcatatgagatcaaattctgagagagcaagatttaaccctttttggttcaccttctgatcctcccctaaggaggtatacatgaaatatttattactcctgcctgaacttctttcattgaatatgcaattttgcagcatgcagattctggatttaaattctgagtcttaacttactggctgagggaccttggataggctccttatccctcagtttcctcatctctaaaatggggatggcacctgccccgtgggttgttggaaggacttacagaggtgcagaatgtacgttgtacatagcaggtttcagcaaatgttagctccctctttccccacatccattcaaatctgttccttctccaaaggatgtgtcaaggaggaaatggacctggctgggaaaccctcagaatactgggatgatgctgagcttggctcatacctgtgctttgctttcaggccagtacggaattc

SEQ ID NO: 3 - DNA, 1285 bp, Homo sapiens
  misc_feature (24)..(24): n is a, c, g, or t
gaattcccaggccctggtattttncttgcaccaagtcctactggtttggcgaggaaagtgatgagaagagccaccctggttccaaccagaagagaatatcagaaagtaagtgctgttgacctcctgctctttctttaacctagtgctgctgcctctgctaactgttgggggcaagcgatgtctcctgcctttctaaaagactgtgaaaccactccaggggcagagaaatcacatgcagtgtccctttccaaatcctcccatgccatttatgtccaatgctgttgacctattgggagttcacggtctcgatccctgagggacattttctttgttgtcttggcttctagaagagtatcttttacttgccccctcccaaacacacatttcatggtctcctaacaagctagaagaaagaggtaaagacaagcgtgattgtggaaccatagcctcgctgcctgcctgtgacatggtgacctgtgtatcagcctgtgtgggctgagaccaagtggctaccacagagctcagcctatgcttcataatgtaatcattacccagatccctaatcctctcttggctcttaactgcagacagagatgtccacagctcatcaaaggctctgccttctgggttctttgtgcttagagtggcttcctaaatatttaataggtcccttttctgccagtctcttctgtgcccatcccctgattgcccttggtaaaagtatgatgccccttagtgtagcacgcttgcctgctgttcctaatcatcttctcctacctcctctttacacctagctcctgtttcagtcacctagaaatgctcacagtcgctggaatatgtcatgttcttccacacctccatgcctttgtaggtactgtttgctctcacaggagaactttctctctaacttgcctatcttctcaactcctcctttctctccaagatctagttccggatcccctcccctgagcatccctccttggttctcaggtagtcagtcactctctgccctgaacttccatggcacgtgaaagaaaatctttttattttaaaacaattacagactcacaagaagtaatacaaattacatgagggggttcccttaaacctttcatccagtttccccaatggtagcagcatgtgtaactgtagaatagtatcaaaaccatgaaattgacataggtacaattcacaaaccttcttcagatttcactagctttatgtgcgctcatttgtgtgtgtgtgtgcgtatttagttctatgcaattttatcatgtgtgaattc

SEQ ID NO: 4 - DNA, 1521 bp, Homo sapiens
gatccctgggccaagggaaggagcacatgaggagttgccgaatgtgaacatgttatctaatcatgagtgtctttccacgtgctagtttgctagatgttatttcttcagcctaaaacaagctggggcctcagatgacctttcccatgtagttcacagaattctgcagtggtcttggaacctgcagccacgaaaagatagattacatatgttggagggagttggtaattcccaggaactctgtctctaagcagatgtgagaagcacctgtgagacgcaatcaagctgggcagctggcttgattgccttccctgcgacctcaaggaccttacagtgggtagtatcaggaggggtcaggggctgtaaagcaccagcgttagcctcagtggcttccagcacgattcctcaaccattctaaccattccaaagggtatatctttggggggtgacattcttttcctgttttctttttaatctttttttaaaacatagaattaatatattatgagcttttcagaagatttttaaaaggcagtcagaaatcctactacctaacacaaaaattgtttttatctttgaataatatgttcttgtttgtccattttccatgcatgcgatgttaggcatacaaaatacattttttaaagaatactttcattgcaaattggaaacttcgtttaaaaaatgctcatactaaaattggcatttctaacccataggcccacttgtagttatttaccgaagcaaaaggacagctttgctttgtgtgggtctggtagggttcattagaaaggaatgggggcggtgggagggttggtgttctgttctctctgcagactgaatggagcatctagagttaagggtaggtcaaccctgacttctgtacttctaaatttttgtcctcaggtcaatcctgaccgggttgttccccccgacctcgggcaccgcctacatcctgggaaaagacattcgctctgagatgagcaccatccggcagaacctgggggtctgtccccagcataacgtgctgtttgacatgtgagtaccagcagcacgttaagaataggccttttctggatgtgtgtgtgtcatgccatcatgggaggagtgggacttaagcattttactttgctgtgtttttgttttttctttttttcttttttatttttttgagatggagtctcgctctgtagccaggctggactgtagtggcgcgatctcggctcactgcaaccttggcctcccaggttcaagcgattctcctgcctcagcctcccgagtagctgggactctaggcacacaccaccatgcccagctaatttttgtgtttttagtagagacggggtttcaccatgttggccaggatggtctcaatgtcttgacctcgtgatccgcccacctcggtctcccaaagtgctgggaacacaggcatgagccactgtgtctggccacattttactttctttgaatatggcaggctcacctccgtgaacaccttgagacctagttgttctttgattttag

SEQ ID NO: 5 - DNA, 6519 bp, Homo sapiens
  misc_feature (3523)..(3523): n is a, c, g, or t
gaaattgaaagttgtaactgcctggtgcatggtggccaggcctgctggaaacaggttggaagcgatctgtcacctttcactttgatttcctgagcagctcatgtggttgctcactgttgttctaccttgaatcttgaagattatttttcagaaattgataaagttattttaaaaagcacggggagagaaaaatatgcccattctcatctgttctgggccaggggacactgtattctggggtatccagtagggcccagagcttgacctgcctccctgtccccaggctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtgcggcccagagctaccttccctatccctctcccctcctcctccggctacacacatgcggaggaaaatcagcactgccccagggtcccaggctgggtgcggttggtaacagaaacttgtccctggctgtgcccctaggtcctctgccttcactcactgtctggggctggtcctggagtttgtcttgctctgtttttttgtaggtggaatgcagagaaagctatctgtggccttggcctttgtcgggggatctaaggttgtcattctggatgaacccacagctggtgtggacccttactcccgcaggggaatatgggagctgctgctgaaataccgacaaggtgcctgatgtgtatttattctgagtaaatggactgagagagagcggggggcttttgagaagtgtggctgtatctcatggctaggcttctgtgaagccatgggatactcttctgttatcacagaagagataaagggcattgagactgagattcctgagaggagatgctgtgtctttattcatctttttgtccccaacatggtgcactaaatttatggttagttgaaagggtggatgcttaaatgaatggaagcggagaggggcaggaagacgattgggctctctggttagagatctgatgtggtacagtatgaggagcacaggcaggcttggagccaactctggctggccctgagacattgggaaagtcacaacttgcctcaccttctttgccgataataatagtggtgcttacctcatagaggattaaattaaatgagaatgcacacaaaccacctagcacaatgcctggcatatagcaagttcccaaataaaatgctactgttcttacctctgtgaggatgtggtacctatatatacaaagctttgccattctaggggtcatagccatacagggtgaaaggtggcttccaggtctcttccagtgcttacccctgctaatatctctctagtccctgtcactgtgacaaatcagaactgagaggcctcacctgtcccacatccttgtgtttgtgcctggcaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggtgagctgcagtcttggtgctgggctggtgttgggtctgggcagccaggacttgctggctgtgaatgatttctccatctccaccccttttgccatgttgaaaccaccatctccctgctctgttgcccctttgaaatcatatcatacttaaggcatggaaagctaaggggccctctgctcccattgtgctagttctgttgaatcccgttttccttttcctatgaggcacagagagtgatggagaaggtccttagaggacattattatgtcaaagaaaagagacttgtcaagaggtaagagccttggctacaaatgacctggtgttcctgctcattacttttcaatctcattgaccttaacttttaaactataaaacagccaatatttattaggcactgatttcatgccagagacactctgggcatgaaagaaagtaatgataatagttaattttatatagcgttgttaccatttacaaccttttttttttttttaacctctatcatctcaattaaagtgcagagagaccctgggaagaaggtaactatatttattatcccagatgagggaagtgaggcttgtagggaattggtagctgattcaaggtcacccagcaggtaaataacagtggtgggaccagacccaattaccaggtatgttttcctctgtaccgcagtacatgcctgagatttatttgtgtgttgaagccagtggtacctaatgtatttacatcccaacctgaaactcctatccacttatttaccttttaatgagcctcttaactcaagtgcagtctgaggaccagcagcatcaggatcacttgggaacttgttagaaattcagcaacctgggcccagctcagacctaccgaatcagaatctgtgcattttaacaaggttcttgagtggttgaacacacattaaagcatgagaagcattgaactagacatgtagccaggtaaaggccttgcctgagatggttggcaaaggcctcattgcagcattcattggcaggccacagttcttttggcagctctgcttcctgacctttcaccctcaggaagcgaggctgttcacacggcacacacatgccagacagggtcctctgaagccacggctgccagtgcatgtgtcccagggaaagctttttcctttagttctcacacaacagagcttcttggaagccctccccggcaaaggtgctggtggctctgccttgctccgtccctgacccgttctcacctccttctttgccatcaggaggacagtgtttctcagagcagttctgatgctggcctgggcagcgaccatgagagtgacacgctgaccatcggtaaggactctggggtttcttattcaggtggtgcctgagcttcccccagctgggcagagtggaggcagaggaggagaggtgcagaggctggtggcgctgactcaaggtttgctgctgggctggggctgggtggctgcgggtgtgggagcagcttggtggcgggttggcctaatgcttgctggggtgcctggggctcggtttgggagctagcagggcagtgtcccagagagctgagatgattggggtttggggaatcccttaggggagtggacactgaataccagggatgaggagctgagggccaagccaggagggtgggatttgagcttagtacataagaagagtgagagcccaggagatgaggaacagccttccagatttttcttgggtagcgtgtgtaggaggccagtgtcaccagtagcatatgtggaacagaagtcttgacccttgctatctctgcctagtcctaatggctggcttttcccaggaaggcttctgcttncatggacngntagattaaccctttatttaggtaaatgagggaacctactttataagcataggaaagggtgaagaatcttttaagattcctttactcaagttttcttttgaagaatcccagagcttaggcaatagacaccagactttgagcctcagttatccattcacccatccacccacccacccacccatccttccatcctcccatcctcccattcacccatccacccatccagctgtccacccattctacactgagtacctataatgtgcctggctttggtgatacaaaggtgaataagacatagtcctttcctttgcccccaaccctcagaccagagatgaacatgtggaatgacctaaacacctggaacaggtgtggtgtatgagcggcaggcctctgatgagagggtgggggatggccagccctcactccgaagcccctctgagttgattgagccatctttgcattctggtccctgcagatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaagtaagttaagtggctgactgtcggaatatatagcaaggccaaatgtcctaaggccagaccagtagcctgcattgggagcaggattatcatggagttagtcattgagtttttaggtcatcgacatctgattaatgttggccccagtgagccatttaagatggtagtgggagatagcaggaaagaagtgttttcctctgtaccacagtacatgcctgagatttgtgtgttgaaaccagtggtacctaacacatttacatcccaaccttaaactcctatgcacttatttaccctttaatgagcctctttacttaagtacagtgtgaggaacagcggcatcaggatcacttgggaacttgttagaaatacagcaacttgggcccagctcagacctactgaatcagaatcaggagcaattctctggtgtgactgtgtcacagccaggtatcaactggattctcatacataggaaatgacaaacgtttatggatggatagtctacttgtgccaggtgctgagatttgttttttgttttttgatttttttttaatcactgtgacctcatttaattctcaaaaaaagatgaaaaaatgaacactcaggaatgctgacatgagattcagaatcaggggtttggggcttcaaagtccatcctctctttatccatgtaatgcctccccttagagatacaacatcacagaccttgaaggctgaaggggatataaaagctgtctggccaagtggtctccaagcttgacagtgcagcagaatcacctggggatattattaaaaataaacatactaaggtttggcttcagggcctgtgaatcagaatttctggaggtgaggccttgaagtctgtatttctattgcatactttggacacagtggtctatagactagagtttggaaatgattgcgctcattcagattctcttctgatgtttgaattgctgccatcatatttctagtgctctatttcctcctgctcattctgtcttggataacttatcatagtactagcctactcaaagatttagagccacagtcctgaaagaagccacttgactcattccctgtaggttcagaataaatttcttctgcgcagtgtctgtcatagctttttttaaatttttttttatttttgatgagactggagttttgctcttattgcccaagctggagtgcagtggtgcgattttggctcactgcaacctccacctcccaggttcaagcgattctcctgcctcagcctcccaagtagctgagattacaagcatgtgctaccacgcccagctaattttgtatttttagtagagatgggttttatccatgttggtcaggctggtctcgagctccagacctcaggtgatctgcccgcctcggcctcccaaagtgctgggattataggcctgagccacagcgctcagccataactttaatttgaaaatgattgtctagcttgatagctctcaccactgaggaaatgttctctggcaaaaacggcttctctcccaggtaactctgagaaagtgttattaagaaatgtggcttctactttctctgtcttacggggctaacatgccactcagtaatataataatcgtggcagtggtgactactctcgtaatgttggtgcttataatgttctcatctctctcattttccagatattcctcaaggtggccgaagagagtggggtggatgctgagacctcaggtaactgccttgagggagaatggcacacttaagatagtgccttctgctggctttctcagtgcacgagtattgttcctttccctttgaattgttctattgcattctcatttgtagagtgtaggtttgttgcagatggggaaggtttgttttgttgtaaataaaataaagtatgggattctttccttgtgccttcagatggtaccttgccagcaagacgaaacaggcgggccttcggggacaagcagagctgtcttcgcccgttcactgaagatgatgctgctgatccaaatgattctgacatagacccaggtctgttagggcaagatcaaacagtgtcctactgtttgaatgtgaaattctctctcatgctctcacctgttttctttggatggcctttanccaaggtgatagatccctacagagtccaaagagaagtgaggaaatggttaaagccacttgttttttgcagcatcgngcatgtnatcaaacctganagagcctatccatatcactttnctttaanagacattaaanatggntccttaatctcttttgancccattgtatttattattctttttctgcgggggtcc

SEQ ID NO: 6 - DNA, 7378 bp, Homo sapiens
tctagaaaatttttaggaacagaaaactttccagttctctcacccctgctcaaagagtgtatggctcttacattatatataactgcctgacttcatacagtatcagtacttagatcatttgaaatgtgtccacgttttaccaaaatataatagggtgagaagctgagatgctaattgccattgtgtattctcaaatatgtcaagctacgtacatggcctgtttcatagagtagtctataagaaattgatgacttgattcatccgaatggctggctgtaacacctggttacgcatgaacacctcttttcagttgtctcaagacacctttcttttctgtacttatcagacaaggactgaaaggcagagactgctactgttagacattttgagtcaagcttttccttggacatagctttgtcatgaaagccctttacttctgagaaacttctagcttcagacacatgccttcaagatagttgttgaagacaccagaagaaggagcatggcaatgccgaaaacacctaagataataggtgaccttcagtgttggcttcttgcagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcaggtgagacgtgctgttttcgccagagactctggcttcatgggtgggctgcaggctctgtgaccagtgaaggcaggatagcatcctggtcaagatatggatgccggagccagatttatctgtatttcaatcccagttctattccttgccagttgtgtatccgctggcaagttacttctctatgcctcaatctcctcatctgtaaaatggggataataatattacctgcaatacagggttgttacgaaaataaaaatgaataggtgcttagaatggggcctgacattagtaagtgcttagttttgtgtgtgtatatgttatttttattttggaggagaacataaaaaggacaaagtgtagaaaaactggttgggtgtattcagctgtcataacatgagagttgttatgcccagatgcacttgacatgtgaatttattagaaacatgatttttctctgagttgatgtttaactcaaactgatagaaaagataggtcagaatatagttggccaacagagaagacttgttagactattgtctgcatgtcagtgtttgcatgctaacttgcttagttagaaaggttaaattttttcactctataaaatcaagaaatatagagaaaaggtctgcagagagtctttcatttgatgatgtggatattgttaagagcgggagtttggagcatacagagctcaagttgaatcctgactttgctacttattggctatatgaccttgggcaagctgcttagtctctctgatcctcagttacctttgtttgttgatgatgaccattgataacacaaccataaataatgacaacatagagatagttctcattatagtagttgttatacagaattattcactcaatgttaattttctgcattgaaatcccagaacattagaattgggggcattatttgaatctttaaggttataaggaatacatttctcagcaataaatggaaggagttttgggttaacttataaagtatacccaagtcatttttttttcagagaagatatggtagaaagtcttaggaggttgaagaaggaattggatatttattctttctgagactatcatgggagataatgactatggttgtccatgattggagccgttgctgtagagttggttttattatagtgtaggatttgaatgggccatgtgttctcagacctcagattaaaatgagaaaactgaggccagtggggagcgtgacttcacatgggtacacttgtgctagagacagaaccaggattcaggacttctggctcctggtcctgggttcatggcccaatgtagtctttctcagtcttcaggaggaggaagggcaggacccagtgttctgagtcaccctgaatgtgagcactatttacttcgtgaacttcttggcttagtgcctctgccaggtggccataacctctggccttgtgttgccagagaaaaggtttagttttcaggctccattgcttcccagctgccaagaatgccttggtgcagcacagtcataggccctgcattcctcattgccgtgctggttggtcggggaggtgggctggactcgtagggatttgccccttggccttgtttctaacacttgccgtttcctgctgtccccctgccccctccactgcctgggtaaagattgtcttgccagctgtgtttgtctgcattgcccttgtgttcagcctgatcgtgccaccctttggcaagtaccccagcctggaacttcagccctggatgtacaacgaacagtacacatttgtcaggtatgtttgtcttctacatcccaggagggggtaagattcgagcagaccaaagatgtttacgagggccaagggaatggacttcagaattacacggtggaatgaattttactgctgcggctcaggtccctgtataagctaatactgcatgcatagaacagcagcgaactaaccctgaataataggccagtcttctgttgagcctttcagcctctctcctcttcatcctactgttgtcaggaacagccacatgtgttttaggtgaaataatccacccttgcaaaaatccatgattaagttataaaatatttggatttgtggagctgtgttttaattctgtaactgagtcacagggcacactgtcaaagcatagaacctccagagacttgttttctgcaaagtataattcatgtaattattatctattctgttatatttgggatgttaggtagtgtttgttctttagataaaaatatcccccactctgtaacaatacattaaatcaaagaaaaggacaaaggatttttctgggtcttgttagcaggagctttcttcagtcctgaaagatttgtagacctgtagatgggggaactgtgtcagtgatacaaaagggaagcatttaaaaaaaaaaagtatatatatatatatatatatatgtaatgtgaattggcctctttttctctaagcccacattttcttcttacatagttcaggtttactttattttttcctttccggctgctgaccctgtattgcccgtagttgtggaacatagcatgtgtttgtgacctgtgcctgttatttttgtgctttctagttgtgcatgcaaagagtacaaagttttcttgccctttcttggaaaatcctgcttgtctgtgccaaagggataattgtgaaagcacttttgaaatacttaatgagttgattttcttcaaattaaaaaaaatatataaatgtatctgtgtatgtacatgtgtgtacacatacacacctttatacatacagcccatttaaaacaagctccactttggagtgctctacgtcaccctgatgccgaatacagggccagagtctgagatccttctgggtggtttctgtgttttgttcatttctgttttaagagcctgtcacagagaaatgcttcctaaaatgtttaatttataaaaacatttttatctctcgattactggttttaatgaattactaagctggctgcctctcatgtacccacagcaatgatgctcctgaggacacgggaaccctggaactcttaaacgccctcaccaaagaccctggcttcgggacccgctgtatggaaggaaacccaatcccgtgagtgccactttagccataagcaggcttcttgtgcttgttgcctggtttgatttctaatatgctgcatttatcaactgcatgccacattgtgaccgccagcatttgccctttgaattattattatgttttatttacaaaaagcgaaggtagtaaccgaactaaattatctaggaacaaacgtttggagagtcttctaacaccgtgcaaagcacgtcattacagacatttgtttactgatttagaaccttaatatttaatttaaatagcactttacacttactgatgaaatgcttttcctttctttctctcccagcccctgtacttaagtgcttcaataggctctcattatatatgatttttaggttttgcttatcagcttcttcgcttttataatctgaaaagatggcatatgaatttttataaaaagggacactttcttcttctcaaattgtatatttttattgtactttccttcaaaacccccttttaaaaagtaagcagtggataaataaattcagtgaagcatccatatgacccttaagtgagtgtaggggaagggaggtcaccagatcactgtgagtgaagatggtggagaggtgaggatcttatgaggccgtgctcaaggctggtagaggtgggttagtgtttccaggtttaggcagaatctcagctgaggtcatgaaacaacagtgatctctgaaaaattatggcaaggtgggaaggtgctggagaattggagagggggcaaacttgactttcaagtttcaatgggaagataggtgactctgcacaccacagaacagtgagcatgataacctgtttatacaaggttctagagcagatttctaaatggatagctactgtgtgcttgtttgttcttaattagtattggatagttactaaatacttgttagtacttagtacataatgggtggtaaatcctagcagctaatattggttcccaaataaccagatgacaaggatagagaaggacacagacacggcctatctggatttcatggtgcctttcattttccacatgaaggttgtgtagggaagatagaagcatgagatgagatgataatatagttatctggattcatcactggccagctgaaccatatgaactcatggattgatgctagcttaggaaggctctgtaggagccagaactgggctgagagccagcccatagagacaaaagaggcccggccctgacatcagagggttcaaacatgatgtctgagccccacctacagtctgccggaggtggttggaaggaagagcctttatccttacaattcttactgaaattcaaatttttaggttttgcaaaaaaatggtggacctgaaggaaatttgacaggagcatgtctcagctgtatttaaatttgtctcagccaatccccttttgaatgttcagagtgtaagcttcaggagggcagcgcgtcttagtgtgacttttctggtcagttcaggtgctttaaggagacaattagagatcaatctggaaaacttcatttgaatttttaatacataagaaaacaataagaaatagttaaaaatatatatttataatatatatatgtgtgtgtgtgtgtgtgtgtgtgtgtgtgtatatatatatattttatttatttatttttttttgagatggagtctcgctctgttgcccaggctggagtgcagtggctcaatcttggctcactgccacctctgcctcccaggttcaagtgattctcctacctcagcctcctgagtagctgggattacaagcatgtgccaccacactggctaatttttctaattttagtagagatggagtttcaccatgttggacaggatggtcttgaactcctgacttagtgatccacccgccttcgcctcccaaagttctgggattacaggcatgagccatcgtgcctggcaattatatttaatatttaataataaggaaataattgctgtaactttactttaaattgtggaattctgaaactggaagggaactggaaatgacttgttgaatcaaatcattttaaacttttattttgccagtggaaaaaataagcccccaaaagagcaggggacctgctgatgtcccacagtaattcagagctggagatgaggttgaaggctttgtgtcttatctccagggaaaatttgtagacagcgtagctctttatgtgacgagcattctcaccccagtcatcccccaattctctactcatttgagaacataaattggatcttgccagtctctactcatttttcagcacatcgagcataagatccagactctttcccaggcctctctcatctggctcctctcctcctcctttatcattactcttcttcgtagcttatcctactccagccatgctgtcttcctattattcctaaaaagtagaaatgcatttcttcctagggcctttgtacctgcacttgccatcgcttttgctcagaatgttctttttgccaagcttttgcccagcttgttctccatcattgttatgttttggctgaaatgtcttctcttagtaggttcattctccccagtcactgtctttttattttgctttattttgggccatctaaggttatcttattagtgtatttgttgttcgtctcctccatgggcatacacctccatgaaggcaggtattttcaccttaggccctcgaatatactggacagcatctggcacgtagtagatgctcaacgaatgtttgttgtgtgagcaaatggttggttgattggattgaactgagttcagtatgtaaatatttagggcctctttgcattctattttacttatgtataaaatgatacataatgatgatataaatgatgtcacagtgtacaaggctgttgtgggatcaagcaatcaaatgagatcatgcttgtcttttccaaatggtgagggaatagatgcatgtttgtggttgttacggaatgatcctgtgctcctgaggcaacagaaaggccaggccatctctggtaatcctactcttgctgtcttccctttgcagagacacgccctgccaggcaggggaggaagagtggacactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaagtgagtcactttcagggggtgattgggcagaaggggtgcaggatgggctggtagcttccgcttggaagcaggaatgagtgagatatcatgttgggagggtctgtttcagtcttttttgttttttgtttttttttctgaggcggagtcttgctctgtcgcccaggctggagtgctgtggcatgatcttgcctcactgcaacctccacctcccaggttcaagcgattctcctgcctcagcctcctgagtagctgggattacaggcacgcaccaccatgtctggctaatttttgtgtttttagtagagatagggtttcgccgtgttggctaggctggtctggaat

SEQ ID NO: 7 - DNA, 5689 bp, Homo sapiens
gaattcctgacctcaggtgatccacccgcctcggcctcccaaagtgctgggattacaggcgtgagccactacgcccagccctgtttcagtctttaactcgcttcttgtcataagaaaaagcatgtgagttttgaggggagaaggtttggaccacactgtgcccatgcctgtcccacagcagtaaagtcacaggacagactgtggcaggcctggcttccaatcttggctctgcaacaaatgagctggtagcctttgacaggcctgggcctgtttcttcacctctgaattagggaggctggaccagaaaactcctgtggatcttgtcaactctggtattcttagagactctgtttgggaaggagtcctgagccattttttttttcttgagaatttcaggaagaggagtgcttatgatagctctctgctgcttttatcagcaaccaaattgcaggatgaggacaagcaattctaaatgagtacaggaactaaaagaaggcttggttaccactcttgaaaataatagctagtccaggtgcggggtggctcacacctgtaatctcagtattttgggatgccgaggtggactgatcacctaaggtcaggagttcgaaaccagcttggccaatgtggcgaaaccctgtctctactaaaaattcaaaaattagccaggcatggtggcacatgcctgtaatcccagttacttgggaggctgaagcaggagaattgcttgaacctgggaggtggaggtcgcagggagccaaaattgcgccactgtactccagcctgagcaacacagcaaaactccatatcaaaaaataaaatgaataaaataacagctaatctagtcatcagtataactccagtgaacagaagatttattaggcatagtgaatgatggtgcttcctaaaaatctcttgactacaaagaatctcatttcaatgtttattgtttagatgttcagaataaattcttgggaaagaccttggcttggtgtaagtgaattaccagtgccgagggcagggtgaaccaagtctcagtgctggttgactgagggcagtgtctgggacctgtagtcaggtttccggtcacactgtggacatggtcactgttgtccttgatttgttttctgtttcaattcttgtctataaagacccgtatgcttggttttcatgtgatgacagagaaaacaaaacactgcagatatccttcaggacctgacaggaagaaacatttcggattatctggtgaagacgtatgtgcagatcatagccaaaaggtgactttttactaaacttggcccctgccgtattattactaattagaggaattaaagacctacaaataacagactgaaacagtgggggaaatgccagattatggcctgattctgtctattggaagtttaggatattatcccaaactagaaaagatgacgagagggactgtgaacattcagttgtcagcttcaaggctgaggcagcctggtctagaatgaaaatagaaatggattcaacgtcaaattttgccacttagtagcaacttgaccaggtaactggttatccttttaaagccttagtttatctaaattgtgatattaatgttgctcttataagtttgtcatgaggactaaattaaatggtgtacatagagtgccttgggtactctctgatgggggactccatgataatttgtggtctcatggagggagctctgggaaggtttaggagcctgccttggctctgcagccttgggagagccttctagcttcccaggacatggcagcctagtgttgaatgcttggctcagcaaatgtttgttctcgtttccttcccatcaacttggtcagttggggtctttcagttaggagtatctcagtgactttaaatggcatgggcatgctggagtgatagtgaccatgagtttctaagaaagaagcataatttctccatatgtcatccacaattgaaatattattgttaattgaaaaagcttctaggccaggcacggtggctcatgcctgtaatcccagcactttaggaggccaaggcgggtggatcacttgaggtcaggagtttgagaccagcctggccaacatggggaaaccctgtctctactaaaaatacaaaataagctgggcgtggtggtgcgtgcctgtaatcccagctacttgggaggctgaggcaggagaattgcttgaatctgggaggcggaggttgcagtgagctgagttcatgccattgcattccagcctgggcaacaagagcgaaaccatctcccaaaagaaaaaaaaaagaaagaaaaagcttctagtttggttacatcttggtctataaggtggtttgtaaattggtttaacccaaggcctggttctcatataagtaatagggtatttatgatggagagaaggctggaagaggcctgaacacaggcttcttttctctagcacaaccctacaaggccagctgattctagggttatttctgtccgttccttatatcctcaggtggatatttactccttttgcatcattaggaataggctcagtgctttctttgaactgattttttgtttctttgtctctgcagcttaaagaacaagatctgggtgaatgagtttaggtaagttgctgtctttctggcacgtttagctcagggggaggatggtgttgtaggtgtcttggattgaagaaagccttggggattgtttgtcactcacacacttgtgggtgccatctcactgtgaggaggacagaagccctgtgaacatgtggagcacacaggggcacagacagatttagattaggcctgctttatagagtttctgcctagagcatcatggctcagtgcccagcagcccctccagaggcctctgaaatatttgatatactgatttccttgaggagaatcagaaatctcctgcaggtgtctagggatttcaagtaagtagtgttgtgaggggaatacctacttgtactttccccccaaaccagattcccgaggcttcttaaggactcaaggacaatttctaggcatttagcacgggactaaaaaggtcttagaggaaataagaagcgccaaaaccatctctttgcactgtatttcaacccatttgtccttctgggttttgaaggaacaggtgggactggggacagaagagttcttgaagccagtttgtccatcatggaaaatgagataggtgatgtggctacgtcagggggcccgaaggctccttgttactgatttccgtcttttctctctgccttttccccaagggccaggacccctggatctctgggcagagcagacgcaggcccctataatagccctcatgctagaaaggagccggagcctgtgtataaggccagcgcagcctactctggacagtgcagggttcccactctcccaactccccatctgcttgcctccagacccacattcacacacgagccactgggttggaggagcatctgtgagatgaaacaccattctttcctcaatgtctcagctatctaactgtgtgtgtaatcaggccaggtcctccctgctgggcagaaaccatgggagttaagagattgccaacatttattagaggaagctgacgtgtaacttctctgaggcaaaatttagccctcctttgaacaggaatttgactcagtgaaccttgtacacactcgcactgagtctgctgctgatgatactgtgcaccccactgtctgggttttaatgtcaggctgttcttttaggtatggcggcttttccctgggtgtcagtaatactcaagcacttcctccgagtcaagaagttaatgatgccatcaaacaaatgaagaaacacctaaagctggccaaggtaaaatatctatcgtaagatgtatcagaaaaatgggcatgtagctgctgggatataggagtagttggcaggttaaacggatcacctggcagctcattgttctgaatatgttggcatacagagccgtctttggcatttagcgatttgagccagacaaaactgaattacttagttgtacgtttaaaagtgtaggtcaaaaacaaatccagaggccaggagctgtggctcatgcctgtaatcctagcactttgggaggccgaagcgggtggatcacttgaggtcaggagttcgagaccagcctggcctacatgacaaaaccccgtatctactaaaaatacaaaaaaattagctgggcttggtggcacacacctgtaatcccagctacttgggaggctgaggcaggagaattgcttgaaccctgtaggaagaggttgtagtgagccaagatcgcaccgttgcactccagcctgggcaacaagagcaaaactccatctcaaaaaacaaattaaatccagagatttaaaagctctcagaggctgggcgcggtggcttacacctgttatcccagcattttgggatgccgaggcgggcaaagcacaaggtcaggagtttgagaccagcctggccaacatagtgaaaccctgtctctgctaaaaacatagaaaaattagccgggcatggtggcgtgcgcctgtaatcccagctactcgggaggctgaggtgagagaattacttgaacccgggaggcggaggttgcagtgagcccagattgcaccactgcactccagcctgggcgacagagcaagactccatctcaaaaaaagctctcagaacaaccaggtttacaaatttggtcagttggtaaataaactgggtttcaaacatactttgctgaaacaatcactgactaaataggaaatgaatcttttttttttttttttaagctggcaagctggtctgtaggacctgataagtactcacttcatttctctgtgtctcaggtttcccatttttaggtgagaattaaggggctctgataaaacagaccctaggattgtggacagcagtgatagtcctagagtccacaagtctgcttttgagtgatgggcccatgtatctggcacatctgcaggcagagcgtggttctggctcttcagatgatgccggtggagcactttgaggagtcctcaccccaccgtgataaccagacattaaaatcttggggctttgcatcccaggatttctctgtgattccttctagacttgtggcatcatggcagcatcactgctgtagatttctagtcacttggttctcaggagccgtttatttaatggcttcacatttaatttcagtgaacaaggtagtggcattgctcttcacagggccgtcctgttgtccacaggttccagattgactgttgccccttatctatgtgaacagtcacaactgaggcaggtttctgttgtttacaggacagttctgcagatcgatttctcaacagcttgggaagatttatgacaggactggacaccaaaaataatgtcaaggtaaaccgctgtctttgttctagtagctttttgatgaacaataatccttatgtttcctggagtactttcaactcatggtaaagttggcaggggcattcacaacagaaaagagcaaactattaactttaccagtgaggcagtacggtgtagtgtagtgattcagagaatttgctttgccaccagacataccaggtaaccttgactaagttacttaacctatctaaacctcagttccctcatctgtgaaatggagacagtaatcatagctatttccaaactgttgtgagaattc

SEQ ID NO: 8 - DNA, 645 bp, Homo sapiens
gaattcaatgagttaaaggtataaggtcctcaccacagcgcctgcccacatagtcagtgatcactatgtcctgaacactgtaattacttcgccatattctctgatcatagtgttttgccttggtatgtgactagaatttctttctgaggtttatgggcatggttggtgggtatgcacctgcctgcaggagcccggtttgggggcattaccttgtacctggtatgttttctttcaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctctgtaagtgtggctgtgtctgtatagatggagtggggcaagggagagggttatggagaaggggagaaaaatgtgaatctcattgtaggggaacagctgcagagaccgttatattatgataaatctggattgatccaggctctgggcagaagtgataagtttacgaattggctggttgggcttcttgaactgcagaagagaaaatgacactgatatgtaaaaatcgtaacatttagtgaattc

SEQ ID NO: 9 - DNA, 1664 bp, Homo sapiens
gaattcatataaagtgagttcaaaaattgttaattaaattataatttaattataagtgtttaatcagtttgatttgtttaaaaaccactgttttaaatttggtggaatatgtttttattagcttgtatctttaattcctaaattaagctgtgtgtgtgtgtgtgtgtgtgtgtgtgtgtgtgtgtgtgtgaagtttaaagccaggatgagctagtttaaagtatgcagcctttggagtcatacagatctgggtttgaatctggtctctaaactttatagatgtatgatattaaatgaggcagttcatgtaaattgccaagcccagcactcagcacagagttgatatttcacacacattagatacctttcctgtatgtggagcatggcagttcctgtttctgctttactcctacaggatactaatataggacactaggatctttataccaagaccccatgtaatgggcttatgagaccattcttcttataaaaatctgacagaatttttgtatgtgttagatcaataggctgcatactgttattttcaagttgatttacagccagaaatattaatttatttgagtagttacagagtaatatttctgctctcatttagttttcaagccccactagtcctttgtgtgtgaaaatttacaacttactgctcttacaaggtcatgaacagtggaccaaagtgaatgccattaaccactctgacttccttcattagttttattgtgacagtggactcttttgacctcagtaataccagtttggcatttacattgtcatatttttagacttaaaaatgatcatcttaaccctgaataaaatgtgtctggtgaacagatgttttttcttgggctgtgcctcagatatctcctgtgtgtgtgtacgtgtgtgtttgtctgtgtgtccatgtcctcactgattgagccctagctgcatcaaaagacccctcagattttcacacgctttttctctccaggatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatggtaaggacacaggcctgctgtatctttctgatgtctgtcagggccatggattgatatggataagaaagaaagagctctggctatcatcaggaaatgttccagctactctaaagatgtatgaaaaagaaatagccagaggcaggtgatcactttcatgacaccaaacacagcattgggtaccagagttcatgtcacaccagagggaaaattctgtacacaatgatgaaaattaataccactaccacttaagttcctatgtgacaactttcccaagaatcagagagatacaagtcaaaactccaagtcaatgcctctaacttctctgatgggttttaacctccagagtcagaatgttctttgccttactaggaaagccatctgtcatttgaaaactctgtacattttatcagcagcttatccatccattgcaaatatgtttttgtgccagccacaatatattgcttctatttggaccaatagggggatttgaaggaattc

SEQ ID NO: 10 - DNA, 1279 bp, Homo sapiens
gaattctcataattgtcctatcgtcaagtctttatttctgcattttactgcttgatacactgtcaggacagactttaaaattattctcagtgcgatgaaacaattctgacattcatgttatgagcagttacctcataaatagattacatgtgagattgaacttgggcagactataatatagcattaatgacgaaacagacacagtcatcttcgggaagaagaatagaggcttatttgctgcctgtgaaattaaaattactctgactgggaatccatcgttcagtaagtttactgagtgtgacaccttggcttgactgttggaaagacagaaagggcatgtagtttataaaatcagccaaggggaaaatgcttgtcaaaatgtattgtcgggtattttgattaatagtttatgtggcttcattaattcagagttactctccaatatgtttatctgccctttcttgtctgataatggtgaaaacttgtgtgatgcattgtatatttgatttaggggtgaactggatgtctttgttttcacttttagtgcaattacgttgtccctgccacactggtcattatcatcttcatctgcttccagcagaagtcctatgtgtcctccaccaatctgcctgtgctagcccttctacttttgctgtatgggtaagtcacctctgagtgagggagctgcacagtggataaggcatttggtgcccagtgtcagaaggagggcagggactctcagtagacacttatctttttgtgtctcaacaggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaatgtgagtcatgcagagagaacactcctgctgggatgagcatctctgggagccagaggacagtgtttaattgtgatcttattccacttgtcagtggtattgacactgctgactgccttgtcctgtcttcagagtctgtcttccctgagaaggcaaagcacctttctttcttgctgtgccttacattttgctggtcaagcctttcagtttcttttgacagttttttttacttctttcttttttcaatgttgctcttaccaagagtagctcctctgccttccactttacacatgagagctgggcgacgccattcagtcctaaggcttttaccatcacctctcttggtgtttttattgtcatctctaa

SEQ ID NO: 11 - DNA, 1124 bp, Homo sapiens
tttaattgattcactaggatatatgctactgaaaggggaatctgcttaaagtgctttctgatatttattattactaaaacttagaatttattaaaaatactgactgtgaaaaattacttgggtcgtttgcctttttaaaaggatttttggcatgtctcattaaaaaaagaaatactagatatcttcagtgaagttacaaatcgaatacacattggctctgaaattctgattgatactgggtcataaaaagttttcccaaatcagacttggaaagtgatcactctcttgttactcttttttccttgtcatgggtgatagccatttgtgtttattggaagatcggtgaattttaaggaacataggcccaaatttgaggaagggccatggtttttgatccctccattctgaccggatctctgcattgtgtctactaggggagaatcgctttgtgtcaccattatcttgggacttggtgggacgaaacctcttcgccatggccgtggaaggggtggtgttcttcctcattactgttctgatccagtacagattcttcatcaggcccaggtgagctttttcttagaacccgtggagcacctggttgagggtcacagaggaggcgcacagggaaacactcaccaatgggggttgcattgaactgaactcaaaatatgtgataaaactgattttcctgatgtgggcatcccgcagccccctccctgcccatcctggagactgtggcaagtaggttttataatactacgttagagactgaatctttgtcctgaaaaatagtttgaaaggttcatttttcttgttttttcccccaagacctgtaaatgcaaagctatctcctctgaatgatgaagatgaagatgtgaggcgggaaagacagagaattcttgatggtggaggccagaatgacatcttagaaatcaaggagttgacgaaggtgagagagtacaggttacaatagctcatcttcagtttttttcagctttatgtgctgtaacccagcagtttgctgacttgcttaataaaagggcatgtgttcccaaaatgtacatctataccaaggttctgtcaattttattttaaaaacaccatggagacttcttaaagaattc

SEQ ID NO: 12 - DNA, 729 bp, Homo sapiens
gaattcccattctcgaatacattggttttatatgcttacatttatgtgttagttattaaaacatactaatattgtatatctagtcaaaactgaggtagagagaataaatggttgattttgagtttgagtttcatagtccaaaaagctgatatattgcctgtgttcaagagggtctatatcagccctctagatgccagcatctccaaattttacttttttggaatctgtacagtatttgcaatatttttattacaaatttctactctgtggaatttaatttttaaaatacctgcaatacatatatatgttgaatagatgaaaaattatgtagataataatgaatgatacggttctaaaaagacaggttaaaaagtaagttcacttttattttgagcttcagaatcattcagaagccagtcgccacaaacgcagaccaaggctcttggcacatcaaatatgcctatggcttagggttattgacaagtcttatgttgcagtgtatgtggtttatagtcctgccttccacagttgcttgggagagctgtgagtcactgaggcttatgaatgtttacattttgtttgttgcagatatatagaaggaagcggaagcctgctgttgacaggatttgcgtgggcattcctcctggtgaggtaaagacactttgtctatattgcgtttgtccctattagttcagactatctctacccaatcaagcaacgatgctcgtt

SEQ ID NO: 13 - DNA, 731 bp, Homo sapiens
acatgtgccagtactggtgagagcgcaagctttggagtcaaacacaaatgggtttgcatcctggccctaccaattatgagctctgagccatgggcaagtgactaactccctgggcctcagtttctctgtaacatctgtcagacttcatgggtccaggtgaggattaaaggagatcatgtatttacagcacatggcatggtgcttcacataaaataagtatttagtaaatgataactggttccttctctcagaaacttatttctgggcctgccaggggccgccctttttcatggcacaagttgggttcccagggttcagtattcttttaaatagttttctggagatcctccatttgggtattttttcctgctttcaggtttggagatggttatacaatagttgtacgaatagcagggtccaacccggacctgaagcctgtccaggatttctttggacttgcatttcctggaagtgttctaaaagagaaacaccggaacatgctacaataccagcttccatcttcattatcttctctggccaggatattcagcatcctctcccagagcaaaaagcgactccacatagaagactactctgtttctcagacaacacttgaccaagtaagctttgagtgtcaaaacagatttacttctcagggtgtggattcctgccccgacactcccgcccataggtccaagagcagtttgtatcttgaattggtgcttgaattc

SEQ ID NO: 14 - DNA, 3501 bp, Homo sapiens
gaattcttcaacagggaaaacagctagcttgaaaacttgctgaaaaacacaacttgtgtttatggcatttagtaccttcaaataattggctttgcagatattggataccccattaaatctgacagtctcaaatttttcatctcttcaatcactagtcaagaaaaatataaaaacaacaaatacttccatatggagcatttttcagagttttctaacccagtcttatttttctagtcagtaaacatttgtaaaaatactgtttcactaatacttactgttaactgtcttgagagaaaagaaaaatatgagagaactattgtttggggaagttcaagtgatctttcaatatcattactaacttcttccactttttccaaaatttgaatattaacgctaaaggtgtaagacttcagatttcaaattaatctttctatattttttaaatttacagaatattatataacccactgctgaaaaagaaaaaaatgattgttttagaagttaaagtcaatattgattttaaatataagtaatgaaggcatatttccaataactagtgatatggcatcgttgcattttacagtatcttcaaaaatacagaatttatagaataatttctcctcatttaatatttttcaaaatcaaagttatggtttcctcattttactaaaatcgtattctaattcttcattatagtaaatctatgagcaactccttacttcggttcctctgatttcaaggccatattttaaaaaatcaaaaggcactgtgaactattttgaagaaaacacaacattttaatacagattgaaaggacctcttctgaagctagaaacaatctatagttatacatcttcattaatactgtgttaccttttaaaatagtaattttttacattttcctgtgtaaacctaattgtggtagaaatttttaccaactctatactcaatcaagcaaaatttctgtatattccctgtggaatgtacctatgtgagtttcagaaattctcaaaatacgtgttcaaaaatttctgcttttgcatctttgggacacctcagaaaacttattaacaactgtgaatatgagaaatacagaagaaaataataagccctctatacataaatgcccagcacaattcattgttaaaaaacaaccaaacctcacactactgtatttcattatctgtactgaaagcaaatgctttgtgactattaaatgttgcacatcattcattcactgtatagtaatcattgactaaagccatttgtctgtgttttcttcttgtggttgtatatatcaggtaaaatattttccaaagagccatgtgtcatgtaatactgaaccactttgatattgagacattaatttgtaccctgtgttattatctactagtaataatgtaatactgtagaaatattgctctaattcttttcaaaattgttgcatcccccttagaatgtttctatttccataaggatttaggtatgctattatcccttcttataccctaagatgaagctgtttttgtgctctttgttcatcattggccctcattccaagcactttacgctgtctgtaatgggatctatttttgcactggaatatctgagaattgcaaaactagacaaaagtttcacaacagattttctaagttaaatcattttcattaaaaggaaaaaagaaaaaaaatttttgtatgtcaataacctttatatgaagtattaaaatgcatatttctatgttgtaatataatgagtcacaaaataaagctgtgacagttctgttggtctacagaaatttacttttgtgcatttgtggcaccacctactgttgaagggttataaagccattagaaaagtagaggggaagtgatttggatcaaaaggaaaaactttagaaaagattcaaatgttcccttaatcataaaagagaactgaggggactacttgaaaataaaaggttgttttgtattttcatgttggttaagatactgagtaactggtattaagtgttagaggtttttagataaatattctgcttaatgattatgaagctgcactgagatttctgaaaatgctctgtagctgagcttatttaataaatgttcacttggtataggggaagctacaaaggcagccttcagtgtccttttgtttattcaaccaaaaatataaggacacaatgtagcagttatactgggaaggtgctgggggtggtggcaatggtgagcaggaaggcgaagtagatatggaaacagaaatgatactaatatcggtgattccttccttttttcctgtaataagtgctgtgcagacaacatatgagcagtgctgataaatgtaaatgtatttttcatagctcattaagaatcagtttcagaaagagatgtctgcttattttgctacttgaagaatccctgtcaaacagtccttttgaggaagtacaagaggctgtctctatttgtgacctcaggaatggctgtgacagtgtcgtgagcagtccttttcctgtggcacagatctgaactttgtgtgcagaaaaatcttggcttcaagtgagccaagatgccccctgagcatcagcatcacaacttcatcctcctatcttgaagttcatgttatagtgactttaatgaaatcatagaacactgtttcttcgtgaacaatgacgagggagaggaaaaaactttattgaaaaataaaaaggcaggtaatttagatgaaaatatgttacccatgaggttttgtttttgctttttgtttttgtttttgagaaacagaatctcgctctgtcgtccaggctggagtgcagcggcatgatcttggctcactgcaacctccgcctcccgggttcaagcgattctcctcagcttcccaagtagctggtactacaggcatgcgccaccacaaccagctaatttttgtatttttagtagagatggggtttcactatacgttggccaggctggtctcaaactcctgacctaaggtgatccttctgccttgggctcccaaagtgctgggattacaggcatgagccaccttgcctggccctacccatgagccttgactaaaacattcttctatctgtagaaaagcccaaaagaacttttccagattcaaaaaacttggcactttgtaatggtaatgtttacattaagtaaaaaaaaaaaaaaaaaacccacttagcttcagttttcaagtgtttactgtgttgtcatgcacttcatttaattctcaacacctgccctatgaggtaaaaagtaccattttacatatgagtaaattacagctcagtggataagaaactcgtccaaaggtacaggttcagtcaagtggcagagggttctttttgttgaagttaggtatcagttaaaattgaccttgtaaaatcacatcagcatcaatatacattaatttaacaaatatttattgaactttactgtatgccagatacttctctaggtactagggggtacaatgtagaagaaaatagaattc

SEQ ID NO: 15 - DNA, 151 bp, Homo sapiens
atgctgttggacagcagggacaatgaccacttttgggaacagcagttggatggcttagattggacagcccaagacatcgtggcgtttttggccaagcacccagaggatgtccagtccagtaatggttctgtgtacacctggagagaagctt

SEQ ID NO: 16 - DNA, 206 bp, Homo sapiens
tgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgg

SEQ ID NO: 17 - DNA, 177 bp, Homo sapiens
gtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacat

SEQ ID NO: 18 - DNA, 223 bp, Homo sapiens
ctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaag

SEQ ID NO: 19 - DNA, 222 bp, Homo sapiens
ttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgct

SEQ ID NO: 20 - DNA, 205 bp, Homo sapiens
agcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccag

SEQ ID NO: 21 - DNA, 15 bp, Homo sapiens
gccagtacggaattc

SEQ ID NO: 22 - DNA, 105 bp, Homo sapiens
  misc_feature (24)..(24): n is a, c, g, or t
gaattcccaggccctggtattttncttgcaccaagtcctactggtttggcgaggaaagtgatgagaagagccaccctggttccaaccagaagagaatatcagaaa

SEQ ID NO: 23 - DNA, 132 bp, Homo sapiens
gtcaatcctgaccgggttgttccccccgacctcgggcaccgcctacatcctgggaaaagacattcgctctgagatgagcaccatccggcagaacctgggggtctgtccccagcataacgtgctgtttgacat

SEQ ID NO: 24 - DNA, 143 bp, Homo sapiens
gctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcag

SEQ ID NO: 25 - DNA, 138 bp, Homo sapiens
gtggaatgcagagaaagctatctgtggccttggcctttgtcgggggatctaaggttgtcattctggatgaacccacagctggtgtggacccttactcccgcaggggaatatgggagctgctgctgaaataccgacaag

SEQ ID NO: 26 - DNA, 221 bp, Homo sapiens
gccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaag

SEQ ID NO: 27 - DNA, 73 bp, Homo sapiens
gaggacagtgtttctcagagcagttctgatgctggcctgggcagcgaccatgagagtgacacgctgaccatcg

SEQ ID NO: 28 - DNA, 203 bp, Homo sapiens
atgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaa

SEQ ID NO: 29 - DNA, 49 bp, Homo sapiens
atattcctcaaggtggccgaagagagtggggtggatgctgagacctcag

SEQ ID NO: 30 - DNA, 114 bp, Homo sapiens
atggtaccttgccagcaagacgaaacaggcgggccttcggggacaagcagagctgtcttcgcccgttcactgaagatgatgctgctgatccaaatgattctgacatagacccag

SEQ ID NO: 31 - DNA, 149 bp, Homo sapiens
aatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcag

SEQ ID NO: 32 - DNA, 125 bp, Homo sapiens
attgtcttgccagctgtgtttgtctgcattgcccttgtgttcagcctgatcgtgccaccctttggcaagtaccccagcctggaacttcagccctggatgtacaacgaacagtacacatttgtcag

SEQ ID NO: 33 - DNA, 99 bp, Homo sapiens
caatgatgctcctgaggacacgggaaccctggaactcttaaacgccctcaccaaagaccctggcttcgggacccgctgtatggaaggaaacccaatccc

SEQ ID NO: 34 - DNA, 189 bp, Homo sapiens
agacacgccctgccaggcaggggaggaagagtggacactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaa

SEQ ID NO: 35 - DNA, 95 bp, Homo sapiens
agaaaacaaaacactgcagatatccttcaggacctgacaggaagaaacatttcggattatctggtgaagacgtatgtgcagatcatagccaaaag

SEQ ID NO: 36 - DNA, 33 bp, Homo sapiens
cttaaagaacaagatctgggtgaatgagtttag

SEQ ID NO: 37 - DNA, 107 bp, Homo sapiens
gtatggcggcttttccctgggtgtcagtaatactcaagcacttcctccgagtcaagaagttaatgatgccatcaaacaaatgaagaaacacctaaagctggccaagg

SEQ ID NO: 38 - DNA, 75 bp, Homo sapiens
gacagttctgcagatcgatttctcaacagcttgggaagatttatgacaggactggacaccaaaaataatgtcaag

SEQ ID NO: 39 - DNA, 170 bp, Homo sapiens
gtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctct

SEQ ID NO: 40 - DNA, 178 bp, Homo sapiens
gatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatg

SEQ ID NO: 41 - DNA, 116 bp, Homo sapiens
tgcaattacgttgtccctgccacactggtcattatcatcttcatctgcttccagcagaagtcctatgtgtcctccaccaatctgcctgtgctagcccttctacttttgctgtatgg

SEQ ID NO: 42 - DNA, 145 bp, Homo sapiens
gtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaat

SEQ ID NO: 43 - DNA, 130 bp, Homo sapiens
gggagaatcgctttgtgtcaccattatcttgggacttggtgggacgaaacctcttcgccatggccgtggaaggggtggtgttcttcctcattactgttctgatccagtacagattcttcatcaggcccag

SEQ ID NO: 44 - DNA, 121 bp, Homo sapiens
acctgtaaatgcaaagctatctcctctgaatgatgaagatgaagatgtgaggcgggaaagacagagaattcttgatggtggaggccagaatgacatcttagaaatcaaggagttgacgaag

SEQ ID NO: 45 - DNA, 63 bp, Homo sapiens
atatatagaaggaagcggaagcctgctgttgacaggatttgcgtgggcattcctcctggtgag

SEQ ID NO: 46 - DNA, 244 bp, Homo sapiens
gtttggagatggttatacaatagttgtacgaatagcagggtccaacccggacctgaagcctgtccaggatttctttggacttgcatttcctggaagtgttctaaaagagaaacaccggaacatgctacaataccagcttccatcttcattatcttctctggccaggatattcagcatcctctcccagagcaaaaagcgactccacatagaagactactctgtttctcagacaacacttgaccaa

SEQ ID NO: 47 - DNA, 1237 bp, Homo sapiens
gaattcttcaacagggaaaacagctagcttgaaaacttgctgaaaaacacaacttgtgtttatggcatttagtaccttcaaataattggctttgcagatattggataccccattaaatctgacagtctcaaatttttcatctcttcaatcactagtcaagaaaaatataaaaacaacaaatacttccatatggagcatttttcagagttttctaacccagtcttatttttctagtcagtaaacatttgtaaaaatactgtttcactaatacttactgttaactgtcttgagagaaaagaaaaatatgagagaactattgtttggggaagttcaagtgatctttcaatatcattactaacttcttccactttttccaaaatttgaatattaacgctaaaggtgtaagacttcagatttcaaattaatctttctatattttttaaatttacagaatattatataacccactgctgaaaaagaaaaaaatgattgttttagaagttaaagtcaatattgattttaaatataagtaatgaaggcatatttccaataactagtgatatggcatcgttgcattttacagtatcttcaaaaatacagaatttatagaataatttctcctcatttaatatttttcaaaatcaaagttatggtttcctcattttactaaaatcgtattctaattcttcattatagtaaatctatgagcaactccttacttcggttcctctgatttcaaggccatattttaaaaaatcaaaaggcactgtgaactattttgaagaaaacacaacattttaatacagattgaaaggacctcttctgaagctagaaacaatctatagttatacatcttcattaatactgtgttaccttttaaaatagtaattttttacattttcctgtgtaaacctaattgtggtagaaatttttaccaactctatactcaatcaagcaaaatttctgtatattccctgtggaatgtacctatgtgagtttcagaaattctcaaaatacgtgttcaaaaatttctgcttttgcatctttgggacacctcagaaaacttattaacaactgtgaatatgagaaatacagaagaaaataataagccctctatacataaatgcccagcacaattcattgttaaaaaacaaccaaacctcacactactgtatttcattatctgtactgaaagcaaatgctttgtgactattaaatgttgcacatcattcattca

SEQ ID NO: 48 - DNA, 3002 bp, Homo sapiens
acatggcaatggcattcattaggaatctagctgggaaaatccagtgtgtatgcttggaaatgagggatctggggctggagagaaaggcatgggcatgccttggagggacttgtgtgtcaagctgaggacctttactttaagctctaggggaccaggcaaggggagatgtagatacgttactctgatggggtggatgaattgaagaaggatgaggcaagaatgaaggcagagaccagggaggaggctctccaagtggccaaggcataaagcaagaaatgaggcctggtgactgcttagtggcagagcagtgaaagagagggaggcatcaaagtgagtctcgatttctagctgggtgggtggtagcgatgtccagtaggccagtggctactgaggtctgcagtggaggagggtggttgggctggagacagatgatgagggagtcatcagcctgtgggtggaagaaaagggaacctcttccaactgttttctttgcttcttccctctctttctctttttttttttttttggacagagtcttgctctgtcacccaggctgaaatgcagtggcatgatcttggctcaccacagcctccgcctcctgggttcaagcaattctcctgtctcagcctccagagtagctgggattacaggcacatatcactgtgcccggctaatttttgtattttcagtggagatgggatttcaccatgttggtcgggctggaatgaactcctgacctcaagtgatccacctgcctcagcctcccaaagtgttgggattacaggcattgagccaccgcgcccggcctttcttccctctcttaaagagtgtttatttaattccacaaacatgagcttgtcaccccctgtagcctggcatctcctacacgaggtgatggctgaggcttctgcttctgctggggtagctctgatctttctgctttctctggcactgtctacccatgttgcctcaccccacaggtcccagggcacctctctcgggcaagtcttggaaccctctgacactgatttgctctcttttctgagctgcttttagccacccatcctcgggacctgttttctctctgcctccacccctgcgggcagtcttaggtctcctgcccctcacgagcaccccagagaggccacgtgctcagtgatctcagtgggcgcatctttctagtcttgctattctttttggccatgttgttcagaaaccatactgggcagggccgacttcaccctaaaggctgcgtctcttcactctgcttttgtttgttccaaataaagtggcttcagaattgctaaccctagcctctgtgaacttgtgaggtacaattttgtgtctgttatgttaacaaaaatacatacataccttcctggtgatggtataaattgctattctctattggaaagcaatttggaatgaaaatttaaagaaccattttaaaatatgctatcctgcgtacctccattccacccacccccagggatgtagcctactgaaataattttaaagaagtcaccatatgagagaaaatgttattgctatattgttattgtgagaaattggaaatagactaaatgttcagcactataggaataattaatgaaattacatatactctatacaatcattatgctgccattgaaataataaatacaaaggcgcaaggggggaaaagcttataatgttagtgaaactaagactgatttttttataaagcagcagttttcagacccttggagactccaattcggtagaaccagagcttcatcttctctgtcgaagctgtgacaggagttgcaaatgcctctcctttttgctgagtttgcagctgctgtttttccggcagcacatctgtgcaggcctctgcctcggcccctctggatctgctgattgagcagcggattgatctgtccttctctttcgtgttgacccatgtgaggaaccaactggcaagggaacaagaaatggaaataggcctcctttgcatcatgacctgtacatcctgcaattggaaaagattgtactttagttggtttaaccagcagcattatttttctaaactaagcagtaagaaggaattaggttttatgtgggatcaacagactgggtctcaaaagaggaaggtgatagaacacagtggggagggggaggtgcactagaaacagagggcctatgctttcattctggctttgctacttaatagctgtgtgacccaatcttagagacttaacctctctgaacttccattttctcatgtataaaatgggaaatattaaaggatactcactgggctggtggcttgtgcctgtaatcccagcacttggggaggttgaggtgggaggatcacttgagcccaggtgttcaagaccagcccaggcaacatggcaagactctgtctctatgaaaaaattaaaaattagccaggtgtggtggtgtgcacctgtagtcttagctacttggtaggctgagatgggaggatcacttgggcttgggaggtcaaggctgcggtgagctgtgattccatcactgcactccagcccgggcggcagagcgagacactgaatccaaacgacaacaacaacaaaaggcaaaaaaataaaagtgccctctttatggagttgtgtaaggtgaagcatatacactattcaacatagtaactatataaaggaagtattgttgttgttactgtagttaataccattaagtgagatgtttcgtatagtggaaagcacatggactctgaattcagactggtctgactttgagtctcagctccacatctagtaatactatgaccaagccctggttaaaatcatgtttttttttcttcagccttagtcttctcacatataaaatagggacactgtcatttacctcagttttctgtgaggataaaacaacgacagtgtatatgcaagtattttgtaaattttgtagtgctcctcaagatttagttggtgtttactacttgtactttctcactggaatggcag

SEQ ID NO: 49 - DNA, 397 bp, Homo sapiens
aattcgggtccaattaaatttttgaaattttatattaaaaattatattagtagggatgggtaagaggtgttttggtctggttggttggttagttgctatgactcagaattgctaagaaaacagaaaagtaagataagatcattgttttaacctcttttcctccacaaaatcaataaataacatatccctaaattactcttagaatttctcttaaattgcagtgaaaaaccaaaatccttcattcttggttgaaggttggaaaactacgttagagaggattagagagagaggatgagcaatcgtgtagtcagcccttgcctcctagtgtaggatttgtctcagccactgcttgttgtcctggctgccaacgttctcatgaaggctgttcttctatcag

SEQ ID NO: 50 - DNA, 520 bp, Homo sapiens
gtaagtggaatcccatcacaccagcctggtcttggggaggtccagagcacctattatattaggacaagaggtactttattttaactaaaaatttggtagaaatttcaacaacaacaaaaaaactcaacttggtgtcatgattttggtgaaattggtacatgacttgctggaaggtttttcataggtcataaaataacagtatcttttgatttagcatttctactcaagggaattaattccaggaattttggtggcaggcacctgtaatcccagctactcgggaggctgaggcaggagaattgcttgaacccaggaggcagaggttgcagtgagctaagatcgcatcattgcactcccgcctgggcaataagagtgaaactccatctcaaaaaaaaaaaaagatacaaaaatagaaaaaggggcttggtaagggtagtagggttttgggcaatttttttttttttttttttttattgtatggttctaaaggaatggttgattacctgtggtttggttttag

SEQ ID NO: 51 - DNA, 1786 bp, Homo sapiens
gtaagttacctgcaagccactgtttttaaccagtttatactgtgccagatgggggtgtatatatgtgtgtgcatgtgcatgcatgtgtgaatgatctggaaataagatgccagatgtaagttgtcaacagttgcagccacatgacagacatagatatatgtgcacacactagtaaacctctttccttctcatccatggttgccacttttatctttttatttttatttttttttttgagatggagtctcgctctgacgcccaggctggagtgcagtggctcgatctcggctcactgcaacctttgcctcccgggttcaagctattctcctgcctcagcctccacagtagctgggactacaggctcatgctgccacgcccggctgactttttgtattttagtagagacgaggtttcaccatgttacccaggctagacttcaactcctgagctcaggcaatccaccctccttggcctcccaaagtgctgggattacaggtgtgagccactgcacccagcccaccactttaattttttacactctacccttttggtcaaaatttgctcaatctgcaagcttaaaatgtgtcatgacaaacacatgcaagcacatactcacacatagatgcagaaacagcgtctaaacttataaaagcacagtttatgtaaatgtgtgcacttcttctccctaggtggtaaaccacatttcaaaacaacccaaataaaactgaacaaagcttcttcctcttagactttttagaaaatctttcagtgctgagtcactaagctgccaagttctcattgtgggaactatgcctttggatgtaatgatttcttctaagacaatgggcggaggtgtagttattgcagacatctgaaatatgtaatgtttcttccagattctggaaattctcttattctctgtggttggtggtggtggtgggatgtgtgtgtgtgtgtgtgtgtgtgtgtgtgtgtgtgtgtagggatcaggatgcgggaggagctgggttctgcttgtattggttctctgttttgcattgaatagtgtgtttccttgtatggctatctatagcttttcaaggtcaccagaaattatcctgtttttcaccttctaaacaattagctggaatttttcaaaggaagacttttacaaagacccctaagctaaggtttactctagaaaggatgtcttaagacagggcacaggagttcagaggcattaagagctggtgcctgttgtcatgtagtgagtatgtgcctacatggtaaagctttgacgtgaacctcaagttcagggtccaaaatctgtgtgcctttttactttgcacatctgcattttctattctagcttggaatctgaaacattgacaagagctgcctgaaatgtatgtctgtggtgtgattagagttacgataagcaagtcaatagtgagatgaccttggagatgttgaacttttgtgagagaatgagttgtttttttgttttggtttttagtactttaacataatctacctttagtttaagtatcgctcacagttacctagttactgaagcaagcccccaaagaaatttggtttggcaacactttgttagcctcgtttttctctctacattgcattgctcgtgaagcattggatcatacgtacatttcagagtctagagggcctgtccttctgtggcccagatgtggtgctccctctagcatgcaggctcagaggccttggcccatcaccctggctcacgtgtgtctttctttctccccttgtccttccttggggcctccag

SEQ ID NO: 52 - DNA, 1745 bp, Homo sapiens
  misc_feature (545)..(545): n is a, c, g, or t
gtaaggcagcctcactcgctcttccctgccaggaaactccgaaatagctcaacacgggctaagggaggagaagaagaaaaaaaatccaagcctctggtagagaaggggtcatacctgtcatttcctgcaatttcatccatttatagttggggaaagtgaggcccagagaggggcagtgacttgcccaaggtcaacccagccgggtagcagctaagtaggatgagagtgcagggttcatgctttccagataaccacatgctcaactgtgccatgctgtctcattggtagtggttcatggcagcatctgaaagctatttattttcttagatatattgggtggcgattcttcctaagtttctaagaacaataatcagaaggatatatattgttgcaggttagactgtctggaagcagaggctgaaatagagtttgatgtatgggtatttatgagggctcaatacctatgaagagatatggaagatgcaggattgggcagagggaggagttgaactgtgatatagggccaaccccgtggggcactctanagaatatgcagcttgttggagttgttnttcatcgagctgaaacatccagccctttgtgctcccccaaggcctccctcctgacaccacctacctcagccctctcaatcaatcactggatgtgggctgccctgggaaggtcgtgccccagggcctacatggctctctgctgctgtgacaaacccagagttgctgatgcctgaggccgtctactgacagctgggcaacaaggcttccctgaatggggactctgggcagtgcagttttgtgtctgaaccatacattaatatatttatatccgaattttctttctctgcaagcatttcatataaagacacatcaggtaaaaataaatgtttttgaagcaaaaggagtacaaagagataagaactaactaatttaatactagttaccatctgttacaaatagttcctactgattgccaaggactgtttaaacacatcacatgggcttcttcttctatcctcactaacccttttaacagacaaggaaatgaggctcaggaaggtcaaggactttattgaggttccacagtaggatacagttcttgctaaaagcaacccctccctcatgctctgttatctaactgcaaggggaaggtcagtggcagaggtagtggtcccatggttggtgcataagagctgctctgagacaactgcatgctggtgggtcctgcagacatgtacccatcagccggagataggctcaaaatatccacaagagtttggatgattgtgggaatgcagaatccatggtgatcaagagggaaagtcaagttgcctggccattttccttggcttttagacagaaaagttacgtgggatattatctcccacagctcttctgtggtgccaccagtcatagtccttatataaggagaaaccagttgaaattacctattgaagaaacaaagagcaaactcgcccactgaaatgcgtagaaagccctggactctgttgtattcataactctgccattatttttctgcgtagttttgggtaagtcacttatcttctttaggatggtaatgatcagttgcctcatcagaaagatgaacagcattacgcctctgcattgtctctaacatgagtaggaataaaccctgtcttttttctgtagatcatacaagtgagtgcttgggattgttgaggcagcacatttgatgtgtctcttccttcccag

SEQ ID NO: 53 - DNA, 1060 bp, Homo sapiens
gtgagtacctctggcctttcttcagtggctgtaggcatttgaccttcctttggagtccctgaataaaagcagcaagttgagaacagaagatgattgtcttttccaatgggacatgaaccttagctctagattctaagctctttaagggtaagggcaagcattgtgttttattaaattgtttacctttagtcttctcagtgaatcctggttgaattgaattgaatggaatttttccgagagccagactgcatcttgaactgggctggggataaatggcattgaggaatggcttcaggcaacagatgccatctctgccctttatctcccagctctgttggctatgttaagctcatgacaaagccaaggccacaaatagaactgaaaactcttgatgtcagagatgacctctcttgtcttccttgtgtccagtatggtgttttgcttgagtaatgttttctgaactaagcacaactgaggagcaggtgcctcatcccacaaattcctgacttggacacttccttccctcgtacagagcagggggatatcttggagagtgtgtgagcccctacaagtgcaagttgtcagatgtccccaggtcacttatcaggaaagctaagagtgactcataggatgctcctgttgcctcagtctgggcttcataggcatcagcagccccaaacaggcacctctgatcctgagccatccttggctgagcagggagcctcagaagactgtgggtatgcgcatgtgtgtgggggaacaggattgctgagccttggggcatctttggaaacataaagttttaaaagttttatgcttcactgtatatgcatttctgaaatgtttgtatataatgagtggttacaaatggaatcattttatatgttacttggtagcccaccactcccctaaagggactctataggtaaatactacttctgcaccttatgattgatccattttgcaaattcaaatttctccaggtataatttacactagaagagatagaaaaatgagactgaccaggaaatggataggtgactttgcctgtttctcacag

SEQ ID NO: 54 - DNA, 1104 bp, Homo sapiens
gtacactgctttgggcatctgtttggaaaatatgacttctagctgatgtcctttctttgtgctagaatctctgcagtgcatgggcttccctgggaagtggtttgggctatagatctatagtaaacagatagtccaaggacaggcagctgatgctgaaagtacaattgtcactacttgtacagcacttgtttcttgaaaactgtgtgccaggcagcatgcaaaatgttttatacacattgcttcatttaattctcacaaggctactctgaagtagttactataataaccagcaattttcaaatgagagaactgtgactcaaagacgttaagtaaccagctttggtcacacaactgttaaatgttggtacgtggaggtgaatccacttcggttacactgggtcaataagcccaggcgaatcctcccaatgctcacccaattctgtatttctgtgtcctcagagggggtacaactaggagaggttctgtttcctgagtacaggttgttaataattaaatatactagctctaaggcctgcctgtgatttaattagcattcaataaaaattcatgttgaatttttctttagtacttctttcttaatataatacatcttcttgaccaagtccaagaggaacctgcgttggacagttttcatatgagatcaaattctgagagagcaagatttaaccctttttggttcaccttctgatcctcccctaaggaggtatacatgaaatatttattactcctgcctgaacttctttcattgaatatgcaattttgcagcatgcagattctggatttaaattctgagtcttaacttactggctgagggaccttggataggctccttatccctcagtttcctcatctctaaaatggggatggcacctgccccgtgggttgttggaaggacttacagaggtgcagaatgtacgttgtacatagcaggtttcagcaaatgttagctccctctttccccacatccattcaaatctgttccttctccaaaggatgtgtcaaggaggaaatggacctggctgggaaaccctcagaatactgggatgatgctgagcttggctcatacctgtgctttgctttcag

SEQ ID NO: 55 - DNA, 1180 bp, Homo sapiens
gtaagtgctgttgacctcctgctctttctttaacctagtgctgctgcctctgctaactgttgggggcaagcgatgtctcctgcctttctaaaagactgtgaaaccactccaggggcagagaaatcacatgcagtgtccctttccaaatcctcccatgccatttatgtccaatgctgttgacctattgggagttcacggtctcgatccctgagggacattttctttgttgtcttggcttctagaagagtatcttttacttgccccctcccaaacacacatttcatggtctcctaacaagctagaagaaagaggtaaagacaagcgtgattgtggaaccatagcctcgctgcctgcctgtgacatggtgacctgtgtatcagcctgtgtgggctgagaccaagtggctaccacagagctcagcctatgcttcataatgtaatcattacccagatccctaatcctctcttggctcttaactgcagacagagatgtccacagctcatcaaaggctctgccttctgggttctttgtgcttagagtggcttcctaaatatttaataggtcccttttctgccagtctcttctgtgcccatcccctgattgcccttggtaaaagtatgatgccccttagtgtagcacgcttgcctgctgttcctaatcatcttctcctacctcctctttacacctagctcctgtttcagtcacctagaaatgctcacagtcgctggaatatgtcatgttcttccacacctccatgcctttgtaggtactgtttgctctcacaggagaactttctctctaacttgcctatcttctcaactcctcctttctctccaagatctagttccggatcccctcccctgagcatccctccttggttctcaggtagtcagtcactctctgccctgaacttccatggcacgtgaaagaaaatctttttattttaaaacaattacagactcacaagaagtaatacaaattacatgagggggttcccttaaacctttcatccagtttccccaatggtagcagcatgtgtaactgtagaatagtatcaaaaccatgaaattgacataggtacaattcacaaaccttcttcagatttcactagctttatgtgcgctcatttgtgtgtgtgtgtgcgtatttagttctatgcaattttatcatgtgtgaattc

SEQ ID NO: 56 - DNA, 903 bp, Homo sapiens
gatccctgggccaagggaaggagcacatgaggagttgccgaatgtgaacatgttatctaatcatgagtgtctttccacgtgctagtttgctagatgttatttcttcagcctaaaacaagctggggcctcagatgacctttcccatgtagttcacagaattctgcagtggtcttggaacctgcagccacgaaaagatagattacatatgttggagggagttggtaattcccaggaactctgtctctaagcagatgtgagaagcacctgtgagacgcaatcaagctgggcagctggcttgattgccttccctgcgacctcaaggaccttacagtgggtagtatcaggaggggtcaggggctgtaaagcaccagcgttagcctcagtggcttccagcacgattcctcaaccattctaaccattccaaagggtatatctttggggggtgacattcttttcctgttttctttttaatctttttttaaaacatagaattaatatattatgagcttttcagaagatttttaaaaggcagtcagaaatcctactacctaacacaaaaattgtttttatctttgaataatatgttcttgtttgtccattttccatgcatgcgatgttaggcatacaaaatacattttttaaagaatactttcattgcaaattggaaacttcgtttaaaaaatgctcatactaaaattggcatttctaacccataggcccacttgtagttatttaccgaagcaaaaggacagctttgctttgtgtgggtctggtagggttcattagaaaggaatgggggcggtgggagggttggtgttctgttctctctgcagactgaatggagcatctagagttaagggtaggtcaaccctgacttctgtacttctaaatttttgtcctcag

SEQ ID NO: 57 - DNA, 486 bp, Homo sapiens
  misc_feature (97)..(97): n is a, c, g, or t
gtgagtaccagcagcacgttaagaataggccttttctggatgtgtgtgtgtcatgccatcatgggaggagtgggacttaagcattttactttgctgngtttttgttttttctttttttcttttttatttttttgagatggagtctcgctctgtagccaggctggactgtagtggcgcgatctcggctcactgcaaccttggcctcccaggttcaagcgattctcctgcctcagcctcccgagtagctgggactctaggcacacaccaccatgcccagctaatttttgtgtttttagtagagacggggtttcaccatgttggccaggatggtctcaatgtcttgacctcgtgatccgcccacctcggtctcccaaagtgctgggaacacaggcatgagccactgtgtctggccacattttactttctttgaatatggcaggctcacctccgtgaacaccttgagacctagttgttctttgattttag

SEQ ID NO: 58 - DNA, 283 bp, Homo sapiens
gaaattgaaagttgtaactgcctggtgcatggtggccaggcctgctggaaacaggttggaagcgatctgtcacctttcactttgatttcctgagcagctcatgtggttgctcactgttgttctaccttgaatcttgaagattatttttcagaaattgataaagttattttaaaaagcacggggagagaaaaatatgcccattctcatctgttctgggccaggggacactgtattctggggtatccagtagggcccagagcttgacctgcctccctgtccccag

SEQ ID NO: 59 - DNA, 203 bp, Homo sapiens
gtgcggcccagagctaccttccctatccctctcccctcctcctccggctacacacatgcggaggaaaatcagcactgccccagggtcccaggctgggtgcggttggtaacagaaacttgtccctggctgtgcccctaggtcctctgccttcactcactgtctggggctggtcctggagtttgtcttgctctgtttttttgtag

SEQ ID NO: 60 - DNA, 702 bp, Homo sapiens
gtgcctgatgtgtatttattctgagtaaatggactgagagagagcggggggcttttgagaagtgtggctgtatctcatggctaggcttctgtgaagccatgggatactcttctgttatcacagaagagataaagggcattgagactgagattcctgagaggagatgctgtgtctttattcatctttttgtccccaacatggtgcactaaatttatggttagttgaaagggtggatgcttaaatgaatggaagcggagaggggcaggaagacgattgggctctctggttagagatctgatgtggtacagtatgaggagcacaggcaggcttggagccaactctggctggccctgagacattgggaaagtcacaacttgcctcaccttctttgccgataataatagtggtgcttacctcatagaggattaaattaaatgagaatgcacacaaaccacctagcacaatgcctggcatatagcaagttcccaaataaaatgctactgttcttacctctgtgaggatgtggtacctatatatacaaagctttgccattctaggggtcatagccatacagggtgaaaggtggcttccaggtctcttccagtgcttacccctgctaatatctctctagtccctgtcactgtgacaaatcagaactgagaggcctcacctgtcccacatccttgtgtttgtgcctggcag

SEQ ID NO: 61 - DNA, 1258 bp, Homo sapiens
gtgagctgcagtcttggtgctgggctggtgttgggtctgggcagccaggacttgctggctgtgaatgatttctccatctccaccccttttgccatgttgaaaccaccatctccctgctctgttgcccctttgaaatcatatcatacttaaggcatggaaagctaaggggccctctgctcccattgtgctagttctgttgaatcccgttttccttttcctatgaggcacagagagtgatggagaaggtccttagaggacattattatgtcaaagaaaagagacttgtcaagaggtaagagccttggctacaaatgacctggtgttcctgctcattacttttcaatctcattgaccttaacttttaaactataaaacagccaatatttattaggcactgatttcatgccagagacactctgggcatgaaagaaagtaatgataatagttaattttatatagcgttgttaccatttacaaccttttttttttttttaacctctatcatctcaattaaagtgcagagagaccctgggaagaaggtaactatatttattatcccagatgagggaagtgaggcttgtagggaattggtagctgattcaaggtcacccagcaggtaaataacagtggtgggaccagacccaattaccaggtatgttttcctctgtaccgcagtacatgcctgagatttatttgtgtgttgaagccagtggtacctaatgtatttacatcccaacctgaaactcctatccacttatttaccttttaatgagcctcttaactcaagtgcagtctgaggaccagcagcatcaggatcacttgggaacttgttagaaattcagcaacctgggcccagctcagacctaccgaatcagaatctgtgcattttaacaaggttcttgagtggttgaacacacattaaagcatgagaagcattgaactagacatgtagccaggtaaaggccttgcctgagatggttggcaaaggcctcattgcagcattcattggcaggccacagttcttttggcagctctgcttcctgacctttcaccctcaggaagcgaggctgttcacacggcacacacatgccagacagggtcctctgaagccacggctgccagtgcatgtgtcccagggaaagctttttcctttagttctcacacaacagagcttcttggaagccctccccggcaaaggtgctggtggctctgccttgctccgtccctgacccgttctcacctccttctttgccatcag

SEQ ID NO: 62 - DNA, 986 bp, Homo sapiens
  misc_feature (502)..(502): n is a, c, g, or t
gtaaggactctggggtttcttattcaggtggtgcctgagcttcccccagctgggcagagtggaggcagaggaggagaggtgcagaggctggtggcgctgactcaaggtttgctgctgggctggggctgggtggctgcgggtgtgggagcagcttggtggcgggttggcctaatgcttgctggggtgcctggggctcggtttgggagctagcagggcagtgtcccagagagctgagatgattggggtttggggaatcccttaggggagtggacactgaataccagggatgaggagctgagggccaagccaggagggtgggatttgagcttagtacataagaagagtgagagcccaggagatgaggaacagccttccagatttttcttgggtagcgtgtgtaggaggccagtgtcaccagtagcatatgtggaacagaagtcttgacccttgctatctctgcctagtcctaatggctggcttttcccaggaaggcttctgcttncatggacngntagattaaccctttatttaggtaaatgagggaacctactttataagcataggaaagggtgaagaatcttttaagattcctttactcaagttttcttttgaagaatcccagagcttaggcaatagacaccagactttgagcctcagttatccattcacccatccacccacccacccacccatccttccatcctcccatcctcccattcacccatccacccatccagctgtccacccattctacactgagtacctataatgtgcctggctttggtgatacaaaggtgaataagacatagtcctttcctttgcccccaaccctcagaccagagatgaacatgtggaatgacctaaacacctggaacaggtgtggtgtatgagcggcaggcctctgatgagagggtgggggatggccagccctcactccgaagcccctctgagttgattgagccatctttgcattctggtccctgcag

SEQ ID NO: 63 - DNA, 1667 bp, Homo sapiens
gtaagttaagtggctgactgtcggaatatatagcaaggccaaatgtcctaaggccagaccagtagcctgcattgggagcaggattatcatggagttagtcattgagtttttaggtcatcgacatctgattaatgttggccccagtgagccatttaagatggtagtgggagatagcaggaaagaagtgttttcctctgtaccacagtacatgcctgagatttgtgtgttgaaaccagtggtacctaacacatttacatcccaaccttaaactcctatgcacttatttaccctttaatgagcctctttacttaagtacagtgtgaggaacagcggcatcaggatcacttgggaacttgttagaaatacagcaacttgggcccagctcagacctactgaatcagaatcaggagcaattctctggtgtgactgtgtcacagccaggtatcaactggattctcatacataggaaatgacaaacgtttatggatggatagtctacttgtgccaggtgctgagatttgttttttgttttttgatttttttttaatcactgtgacctcatttaattctcaaaaaaagatgaaaaaatgaacactcaggaatgctgacatgagattcagaatcaggggtttggggcttcaaagtccatcctctctttatccatgtaatgcctccccttagagatacaacatcacagaccttgaaggctgaaggggatataaaagctgtctggccaagtggtctccaagcttgacagtgcagcagaatcacctggggatattattaaaaataaacatactaaggtttggcttcagggcctgtgaatcagaatttctggaggtgaggccttgaagtctgtatttctattgcatactttggacacagtggtctatagactagagtttggaaatgattgcgctcattcagattctcttctgatgtttgaattgctgccatcatatttctagtgctctatttcctcctgctcattctgtcttggataacttatcatagtactagcctactcaaagatttagagccacagtcctgaaagaagccacttgactcattccctgtaggttcagaataaatttcttctgcgcagtgtctgtcatagctttttttaaatttttttttatttttgatgagactggagttttgctcttattgcccaagctggagtgcagtggtgcgattttggctcactgcaacctccacctcccaggttcaagcgattctcctgcctcagcctcccaagtagctgagattacaagcatgtgctaccacgcccagctaattttgtatttttagtagagatgggttttatccatgttggtcaggctggtctcgagctccagacctcaggtgatctgcccgcctcggcctcccaaagtgctgggattataggcctgagccacagcgctcagccataactttaatttgaaaatgattgtctagcttgatagctctcaccactgaggaaatgttctctggcaaaaacggcttctctcccaggtaactctgagaaagtgttattaagaaatgtggcttctactttctctgtcttacggggctaacatgccactcagtaatataataatcgtggcagtggtgactactctcgtaatgttggtgcttataatgttctcatctctctcattttccag

SEQ ID NO: 64 - DNA, 195 bp, Homo sapiens
gtaactgccttgagggagaatggcacacttaagatagtgccttctgctggctttctcagtgcacgagtattgttcctttccctttgaattgttctattgcattctcatttgtagagtgtaggtttgttgcagatggggaaggtttgttttgttgtaaataaaataaagtatgggattctttccttgtgccttcag

SEQ ID NO: 65 - DNA, 284 bp, Homo sapiens
  misc_feature (90)..(90): n is a, c, g, or t
gtctgttagggcaagatcaaacagtgtcctactgtttgaatgtgaaattctctctcatgctctcacctgttttctttggatggcctttanccaaggtgatagatccctacagagtccaaagagaagtgaggaaatggttaaagccacttgttttttgcagcatcgngcatgtnatcaaacctganagagcctatccatatcactttnctttaanagacattaaanatggntccttaatctcttttgancccattgtatttattattctttttctgcgggggtcc

SEQ ID NO: 66 - DNA, 560 bp, Homo sapiens
tctagaaaatttttaggaacagaaaactttccagttctctcacccctgctcaaagagtgtatggctcttacattatatataactgcctgacttcatacagtatcagtacttagatcatttgaaatgtgtccacgttttaccaaaatataatagggtgagaagctgagatgctaattgccattgtgtattctcaaatatgtcaagctacgtacatggcctgtttcatagagtagtctataagaaattgatgacttgattcatccgaatggctggctgtaacacctggttacgcatgaacacctcttttcagttgtctcaagacacctttcttttctgtacttatcagacaaggactgaaaggcagagactgctactgttagacattttgagtcaagcttttccttggacatagctttgtcatgaaagccctttacttctgagaaacttctagcttcagacacatgccttcaagatagttgttgaagacaccagaagaaggagcatggcaatgccgaaaacacctaagataataggtgaccttcagtgttggcttcttgcag

SEQ ID NO: 67 - DNA, 1649 bp, Homo sapiens
gtgagacgtgctgttttcgccagagactctggcttcatgggtgggctgcaggctctgtgaccagtgaaggcaggatagcatcctggtcaagatatggatgccggagccagatttatctgtatttcaatcccagttctattccttgccagttgtgtatccgctggcaagttacttctctatgcctcaatctcctcatctgtaaaatggggataataatattacctgcaatacagggttgttacgaaaataaaaatgaataggtgcttagaatggggcctgacattagtaagtgcttagttttgtgtgtgtatatgttatttttattttggaggagaacataaaaaggacaaagtgtagaaaaactggttgggtgtattcagctgtcataacatgagagttgttatgcccagatgcacttgacatgtgaatttattagaaacatgatttttctctgagttgatgtttaactcaaactgatagaaaagataggtcagaatatagttggccaacagagaagacttgttagactattgtctgcatgtcagtgtttgcatgctaacttgcttagttagaaaggttaaattttttcactctataaaatcaagaaatatagagaaaaggtctgcagagagtctttcatttgatgatgtggatattgttaagagcgggagtttggagcatacagagctcaagttgaatcctgactttgctacttattggctatatgaccttgggcaagctgcttagtctctctgatcctcagttacctttgtttgttgatgatgaccattgataacacaaccataaataatgacaacatagagatagttctcattatagtagttgttatacagaattattcactcaatgttaattttctgcattgaaatcccagaacattagaattgggggcattatttgaatctttaaggttataaggaatacatttctcagcaataaatggaaggagttttgggttaacttataaagtatacccaagtcatttttttttcagagaagatatggtagaaagtcttaggaggttgaagaaggaattggatatttattctttctgagactatcatgggagataatgactatggttgtccatgattggagccgttgctgtagagttggttttattatagtgtaggatttgaatgggccatgtgttctcagacctcagattaaaatgagaaaactgaggccagtggggagcgtgacttcacatgggtacacttgtgctagagacagaaccaggattcaggacttctggctcctggtcctgggttcatggcccaatgtagtctttctcagtcttcaggaggaggaagggcaggacccagtgttctgagtcaccctgaatgtgagcactatttacttcgtgaacttcttggcttagtgcctctgccaggtggccataacctctggccttgtgttgccagagaaaaggtttagttttcaggctccattgcttcccagctgccaagaatgccttggtgcagcacagtcataggccctgcattcctcattgccgtgctggttggtcggggaggtgggctggactcgtagggatttgccccttggccttgtttctaacacttgccgtttcctgctgtccccctgccccctccactgcctgggtaaag

SEQ ID NO: 68 - DNA, 1230 bp, Homo sapiens
gtatgtttgtcttctacatcccaggagggggtaagattcgagcagaccaaagatgtttacgagggccaagggaatggacttcagaattacacggtggaatgaattttactgctgcggctcaggtccctgtataagctaatactgcatgcatagaacagcagcgaactaaccctgaataataggccagtcttctgttgagcctttcagcctctctcctcttcatcctactgttgtcaggaacagccacatgtgttttaggtgaaataatccacccttgcaaaaatccatgattaagttataaaatatttggatttgtggagctgtgttttaattctgtaactgagtcacagggcacactgtcaaagcatagaacctccagagacttgttttctgcaaagtataattcatgtaattattatctattctgttatatttgggatgttaggtagtgtttgttctttagataaaaatatcccccactctgtaacaatacattaaatcaaagaaaaggacaaaggatttttctgggtcttgttagcaggagctttcttcagtcctgaaagatttgtagacctgtagatgggggaactgtgtcagtgatacaaaagggaagcatttaaaaaaaaaaagtatatatatatatatatatatatgtaatgtgaattggcctctttttctctaagcccacattttcttcttacatagttcaggtttactttattttttcctttccggctgctgaccctgtattgcccgtagttgtggaacatagcatgtgtttgtgacctgtgcctgttatttttgtgctttctagttgtgcatgcaaagagtacaaagttttcttgccctttcttggaaaatcctgcttgtctgtgccaaagggataattgtgaaagcacttttgaaatacttaatgagttgattttcttcaaattaaaaaaaatatataaatgtatctgtgtatgtacatgtgtgtacacatacacacctttatacatacagcccatttaaaacaagctccactttggagtgctctacgtcaccctgatgccgaatacagggccagagtctgagatccttctgggtggtttctgtgttttgttcatttctgttttaagagcctgtcacagagaaatgcttcctaaaatgtttaatttataaaaacatttttatctctcgattactggttttaatgaattactaagctggctgcctctcatgtacccacag

SEQ ID NO: 69 - DNA, 3035 bp, Homo sapiens
gtgagtgccactttagccataagcaggcttcttgtgcttgttgcctggtttgatttctaatatgctgcatttatcaactgcatgccacattgtgaccgccagcatttgccctttgaattattattatgttttatttacaaaaagcgaaggtagtaaccgaactaaattatctaggaacaaacgtttggagagtcttctaacaccgtgcaaagcacgtcattacagacatttgtttactgatttagaaccttaatatttaatttaaatagcactttacacttactgatgaaatgcttttcctttctttctctcccagcccctgtacttaagtgcttcaataggctctcattatatatgatttttaggttttgcttatcagcttcttcgcttttataatctgaaaagatggcatatgaatttttataaaaagggacactttcttcttctcaaattgtatatttttattgtactttccttcaaaacccccttttaaaaagtaagcagtggataaataaattcagtgaagcatccatatgacccttaagtgagtgtaggggaagggaggtcaccagatcactgtgagtgaagatggtggagaggtgaggatcttatgaggccgtgctcaaggctggtagaggtgggttagtgtttccaggtttaggcagaatctcagctgaggtcatgaaacaacagtgatctctgaaaaattatggcaaggtgggaaggtgctggagaattggagagggggcaaacttgactttcaagtttcaatgggaagataggtgactctgcacaccacagaacagtgagcatgataacctgtttatacaaggttctagagcagatttctaaatggatagctactgtgtgcttgtttgttcttaattagtattggatagttactaaatacttgttagtacttagtacataatgggtggtaaatcctagcagctaatattggttcccaaataaccagatgacaaggatagagaaggacacagacacggcctatctggatttcatggtgcctttcattttccacatgaaggttgtgtagggaagatagaagcatgagatgagatgataatatagttatctggattcatcactggccagctgaaccatatgaactcatggattgatgctagcttaggaaggctctgtaggagccagaactgggctgagagccagcccatagagacaaaagaggcccggccctgacatcagagggttcaaacatgatgtctgagccccacctacagtctgccggaggtggttggaaggaagagcctttatccttacaattcttactgaaattcaaatttttaggttttgcaaaaaaatggtggacctgaaggaaatttgacaggagcatgtctcagctgtatttaaatttgtctcagccaatccccttttgaatgttcagagtgtaagcttcaggagggcagcgcgtcttagtgtgacttttctggtcagttcaggtgctttaaggagacaattagagatcaatctggaaaacttcatttgaatttttaatacataagaaaacaataagaaatagttaaaaatatatatttataatatatatatgtgtgtgtgtgtgtgtgtgtgtgtgtgtgtatatatatatattttatttatttatttttttttgagatggagtctcgctctgttgcccaggctggagtgcagtggctcaatcttggctcactgccacctctgcctcccaggttcaagtgattctcctacctcagcctcctgagtagctgggattacaagcatgtgccaccacactggctaatttttctaattttagtagagatggagtttcaccatgttggacaggatggtcttgaactcctgacttagtgatccacccgccttcgcctcccaaagttctgggattacaggcatgagccatcgtgcctggcaattatatttaatatttaataataaggaaataattgctgtaactttactttaaattgtggaattctgaaactggaagggaactggaaatgacttgttgaatcaaatcattttaaacttttattttgccagtggaaaaaataagcccccaaaagagcaggggacctgctgatgtcccacagtaattcagagctggagatgaggttgaaggctttgtgtcttatctccagggaaaatttgtagacagcgtagctctttatgtgacgagcattctcaccccagtcatcccccaattctctactcatttgagaacataaattggatcttgccagtctctactcatttttcagcacatcgagcataagatccagactctttcccaggcctctctcatctggctcctctcctcctcctttatcattactcttcttcgtagcttatcctactccagccatgctgtcttcctattattcctaaaaagtagaaatgcatttcttcctagggcctttgtacctgcacttgccatcgcttttgctcagaatgttctttttgccaagcttttgcccagcttgttctccatcattgttatgttttggctgaaatgtcttctcttagtaggttcattctccccagtcactgtctttttattttgctttattttgggccatctaaggttatcttattagtgtatttgttgttcgtctcctccatgggcatacacctccatgaaggcaggtattttcaccttaggccctcgaatatactggacagcatctggcacgtagtagatgctcaacgaatgtttgttgtgtgagcaaatggttggttgattggattgaactgagttcagtatgtaaatatttagggcctctttgcattctattttacttatgtataaaatgatacataatgatgatataaatgatgtcacagtgtacaaggctgttgtgggatcaagcaatcaaatgagatcatgcttgtcttttccaaatggtgagggaatagatgcatgtttgtggttgttacggaatgatcctgtgctcctgaggcaacagaaaggccaggccatctctggtaatcctactcttgctgtcttccctttgcag

SEQ ID NO: 70 - DNA, 342 bp, Homo sapiens
gtgagtcactttcagggggtgattgggcagaaggggtgcaggatgggctggtagcttccgcttggaagcaggaatgagtgagatatcatgttgggagggtctgtttcagtcttttttgttttttgtttttttttctgaggcggagtcttgctctgtcgcccaggctggagtgctgtggcatgatcttgcctcactgcaacctccacctcccaggttcaagcgattctcctgcctcagcctcctgagtagctgggattacaggcacgcaccaccatgtctggctaatttttgtgtttttagtagagatagggtttcgccgtgttggctaggctggtctggaat

SEQ ID NO: 71 - DNA, 1182 bp, Homo sapiens
gaattcctgacctcaggtgatccacccgcctcggcctcccaaagtgctgggattacaggcgtgagccactacgcccagccctgtttcagtctttaactcgcttcttgtcataagaaaaagcatgtgagttttgaggggagaaggtttggaccacactgtgcccatgcctgtcccacagcagtaaagtcacaggacagactgtggcaggcctggcttccaatcttggctctgcaacaaatgagctggtagcctttgacaggcctgggcctgtttcttcacctctgaattagggaggctggaccagaaaactcctgtggatcttgtcaactctggtattcttagagactctgtttgggaaggagtcctgagccattttttttttcttgagaatttcaggaagaggagtgcttatgatagctctctgctgcttttatcagcaaccaaattgcaggatgaggacaagcaattctaaatgagtacaggaactaaaagaaggcttggttaccactcttgaaaataatagctagtccaggtgcggggtggctcacacctgtaatctcagtattttgggatgccgaggtggactgatcacctaaggtcaggagttcgaaaccagcttggccaatgtggcgaaaccctgtctctactaaaaattcaaaaattagccaggcatggtggcacatgcctgtaatcccagttacttgggaggctgaagcaggagaattgcttgaacctgggaggtggaggtcgcagggagccaaaattgcgccactgtactccagcctgagcaacacagcaaaactccatatcaaaaaataaaatgaataaaataacagctaatctagtcatcagtataactccagtgaacagaagatttattaggcatagtgaatgatggtgcttcctaaaaatctcttgactacaaagaatctcatttcaatgtttattgtttagatgttcagaataaattcttgggaaagaccttggcttggtgtaagtgaattaccagtgccgagggcagggtgaaccaagtctcagtgctggttgactgagggcagtgtctgggacctgtagtcaggtttccggtcacactgtggacatggtcactgttgtccttgatttgttttctgtttcaattcttgtctataaagacccgtatgcttggttttcatgtgatgacag

SEQ ID NO: 72 - DNA, 1309 bp, Homo sapiens
gtgactttttactaaacttggcccctgccgtattattactaattagaggaattaaagacctacaaataacagactgaaacagtgggggaaatgccagattatggcctgattctgtctattggaagtttaggatattatcccaaactagaaaagatgacgagagggactgtgaacattcagttgtcagcttcaaggctgaggcagcctggtctagaatgaaaatagaaatggattcaacgtcaaattttgccacttagtagcaacttgaccaggtaactggttatccttttaaagccttagtttatctaaattgtgatattaatgttgctcttataagtttgtcatgaggactaaattaaatggtgtacatagagtgccttgggtactctctgatgggggactccatgataatttgtggtctcatggagggagctctgggaaggtttaggagcctgccttggctctgcagccttgggagagccttctagcttcccaggacatggcagcctagtgttgaatgcttggctcagcaaatgtttgttctcgtttccttcccatcaacttggtcagttggggtctttcagttaggagtatctcagtgactttaaatggcatgggcatgctggagtgatagtgaccatgagtttctaagaaagaagcataatttctccatatgtcatccacaattgaaatattattgttaattgaaaaagcttctaggccaggcacggtggctcatgcctgtaatcccagcactttaggaggccaaggcgggtggatcacttgaggtcaggagtttgagaccagcctggccaacatggggaaaccctgtctctactaaaaatacaaaataagctgggcgtggtggtgcgtgcctgtaatcccagctacttgggaggctgaggcaggagaattgcttgaatctgggaggcggaggttgcagtgagctgagttcatgccattgcattccagcctgggcaacaagagcgaaaccatctcccaaaagaaaaaaaaaagaaagaaaaagcttctagtttggttacatcttggtctataaggtggtttgtaaattggtttaacccaaggcctggttctcatataagtaatagggtatttatgatggagagaaggctggaagaggcctgaacacaggcttcttttctctagcacaaccctacaaggccagctgattctagggttatttctgtccgttccttatatcctcaggtggatatttactccttttgcatcattaggaataggctcagtgctttctttgaactgattttttgtttctttgtctctgcag

SEQ ID NO: 73 - DNA, 1124 bp, Homo sapiens
gtaagttgctgtctttctggcacgtttagctcagggggaggatggtgttgtaggtgtcttggattgaagaaagccttggggattgtttgtcactcacacacttgtgggtgccatctcactgtgaggaggacagaagccctgtgaacatgtggagcacacaggggcacagacagatttagattaggcctgctttatagagtttctgcctagagcatcatggctcagtgcccagcagcccctccagaggcctctgaaatatttgatatactgatttccttgaggagaatcagaaatctcctgcaggtgtctagggatttcaagtaagtagtgttgtgaggggaatacctacttgtactttccccccaaaccagattcccgaggcttcttaaggactcaaggacaatttctaggcatttagcacgggactaaaaaggtcttagaggaaataagaagcgccaaaaccatctctttgcactgtatttcaacccatttgtccttctgggttttgaaggaacaggtgggactggggacagaagagttcttgaagccagtttgtccatcatggaaaatgagataggtgatgtggctacgtcagggggcccgaaggctccttgttactgatttccgtcttttctctctgccttttccccaagggccaggacccctggatctctgggcagagcagacgcaggcccctataatagccctcatgctagaaaggagccggagcctgtgtataaggccagcgcagcctactctggacagtgcagggttcccactctcccaactccccatctgcttgcctccagacccacattcacacacgagccactgggttggaggagcatctgtgagatgaaacaccattctttcctcaatgtctcagctatctaactgtgtgtgtaatcaggccaggtcctccctgctgggcagaaaccatgggagttaagagattgccaacatttattagaggaagctgacgtgtaacttctctgaggcaaaatttagccctcctttgaacaggaatttgactcagtgaaccttgtacacactcgcactgagtctgctgctgatgatactgtgcaccccactgtctgggttttaatgtcaggctgttcttttag

SEQ ID NO: 74 - DNA, 1473 bp, Homo sapiens
gtaaaatatctatcgtaagatgtatcagaaaaatgggcatgtagctgctgggatataggagtagttggcaggttaaacggatcacctggcagctcattgttctgaatatgttggcatacagagccgtctttggcatttagcgatttgagccagacaaaactgaattacttagttgtacgtttaaaagtgtaggtcaaaaacaaatccagaggccaggagctgtggctcatgcctgtaatcctagcactttgggaggccgaagcgggtggatcacttgaggtcaggagttcgagaccagcctggcctacatgacaaaaccccgtatctactaaaaatacaaaaaaattagctgggcttggtggcacacacctgtaatcccagctacttgggaggctgaggcaggagaattgcttgaaccctgtaggaagaggttgtagtgagccaagatcgcaccgttgcactccagcctgggcaacaagagcaaaactccatctcaaaaaacaaattaaatccagagatttaaaagctctcagaggctgggcgcggtggcttacacctgttatcccagcattttgggatgccgaggcgggcaaagcacaaggtcaggagtttgagaccagcctggccaacatagtgaaaccctgtctctgctaaaaacatagaaaaattagccgggcatggtggcgtgcgcctgtaatcccagctactcgggaggctgaggtgagagaattacttgaacccgggaggcggaggttgcagtgagcccagattgcaccactgcactccagcctgggcgacagagcaagactccatctcaaaaaaagctctcagaacaaccaggtttacaaatttggtcagttggtaaataaactgggtttcaaacatactttgctgaaacaatcactgactaaataggaaatgaatcttttttttttttttttaagctggcaagctggtctgtaggacctgataagtactcacttcatttctctgtgtctcaggtttcccatttttaggtgagaattaaggggctctgataaaacagaccctaggattgtggacagcagtgatagtcctagagtccacaagtctgcttttgagtgatgggcccatgtatctggcacatctgcaggcagagcgtggttctggctcttcagatgatgccggtggagcactttgaggagtcctcaccccaccgtgataaccagacattaaaatcttggggctttgcatcccaggatttctctgtgattccttctagacttgtggcatcatggcagcatcactgctgtagatttctagtcacttggttctcaggagccgtttatttaatggcttcacatttaatttcagtgaacaaggtagtggcattgctcttcacagggccgtcctgttgtccacaggttccagattgactgttgccccttatctatgtgaacagtcacaactgaggcaggtttctgttgtttacag

SEQ ID NO: 75 - DNA, 292 bp, Homo sapiens
gtaaaccgctgtctttgttctagtagctttttgatgaacaataatccttatgtttcctggagtactttcaactcatggtaaagttggcaggggcattcacaacagaaaagagcaaactattaactttaccagtgaggcagtacggtgtagtgtagtgattcagagaatttgctttgccaccagacataccaggtaaccttgactaagttacttaacctatctaaacctcagttccctcatctgtgaaatggagacagtaatcatagctatttccaaactgttgtgagaattc

SEQ ID NO: 76 - DNA, 235 bp, Homo sapiens
gaattcaatgagttaaaggtataaggtcctcaccacagcgcctgcccacatagtcagtgatcactatgtcctgaacactgtaattacttcgccatattctctgatcatagtgttttgccttggtatgtgactagaatttctttctgaggtttatgggcatggttggtgggtatgcacctgcctgcaggagcccggtttgggggcattaccttgtacctggtatgttttctttcag

SEQ ID NO: 77 - DNA, 240 bp, Homo sapiens
gtaagtgtggctgtgtctgtatagatggagtggggcaagggagagggttatggagaaggggagaaaaatgtgaatctcattgtaggggaacagctgcagagaccgttatattatgataaatctggattgatccaggctctgggcagaagtgataagtttacgaattggctggttgggcttcttgaactgcagaagagaaaatgacactgatatgtaaaaatcgtaacatttagtgaattc

SEQ ID NO: 78 - DNA, 988 bp, Homo sapiens
gaattcatataaagtgagttcaaaaattgttaattaaattataatttaattataagtgtttaatcagtttgatttgtttaaaaaccactgttttaaatttggtggaatatgtttttattagcttgtatctttaattcctaaattaagctgtgtgtgtgtgtgtgtgtgtgtgtgtgtgtgtgtgtgtgtgaagtttaaagccaggatgagctagtttaaagtatgcagcctttggagtcatacagatctgggtttgaatctggtctctaaactttatagatgtatgatattaaatgaggcagttcatgtaaattgccaagcccagcactcagcacagagttgatatttcacacacattagatacctttcctgtatgtggagcatggcagttcctgtttctgctttactcctacaggatactaatataggacactaggatctttataccaagaccccatgtaatgggcttatgagaccattcttcttataaaaatctgacagaatttttgtatgtgttagatcaataggctgcatactgttattttcaagttgatttacagccagaaatattaatttatttgagtagttacagagtaatatttctgctctcatttagttttcaagccccactagtcctttgtgtgtgaaaatttacaacttactgctcttacaaggtcatgaacagtggaccaaagtgaatgccattaaccactctgacttccttcattagttttattgtgacagtggactcttttgacctcagtaataccagtttggcatttacattgtcatatttttagacttaaaaatgatcatcttaaccctgaataaaatgtgtctggtgaacagatgttttttcttgggctgtgcctcagatatctcctgtgtgtgtgtacgtgtgtgtttgtctgtgtgtccatgtcctcactgattgagccctagctgcatcaaaagacccctcagattttcacacgctttttctctccag

SEQ ID NO: 79 - DNA, 498 bp, Homo sapiens
gtaaggacacaggcctgctgtatctttctgatgtctgtcagggccatggattgatatggataagaaagaaagagctctggctatcatcaggaaatgttccagctactctaaagatgtatgaaaaagaaatagccagaggcaggtgatcactttcatgacaccaaacacagcattgggtaccagagttcatgtcacaccagagggaaaattctgtacacaatgatgaaaattaataccactaccacttaagttcctatgtgacaactttcccaagaatcagagagatacaagtcaaaactccaagtcaatgcctctaacttctctgatgggttttaacctccagagtcagaatgttctttgccttactaggaaagccatctgtcatttgaaaactctgtacattttatcagcagcttatccatccattgcaaatatgtttttgtgccagccacaatatattgcttctatttggaccaatagggggatttgaaggaattc

SEQ ID NO: 80 - DNA, 544 bp, Homo sapiens
gaattctcataattgtcctatcgtcaagtctttatttctgcattttactgcttgatacactgtcaggacagactttaaaattattctcagtgcgatgaaacaattctgacattcatgttatgagcagttacctcataaatagattacatgtgagattgaacttgggcagactataatatagcattaatgacgaaacagacacagtcatcttcgggaagaagaatagaggcttatttgctgcctgtgaaattaaaattactctgactgggaatccatcgttcagtaagtttactgagtgtgacaccttggcttgactgttggaaagacagaaagggcatgtagtttataaaatcagccaaggggaaaatgcttgtcaaaatgtattgtcgggtattttgattaatagtttatgtggcttcattaattcagagttactctccaatatgtttatctgccctttcttgtctgataatggtgaaaacttgtgtgatgcattgtatatttgatttaggggtgaactggatgtctttgttttcacttttag

SEQ ID NO: 81 - DNA, 111 bp, Homo sapiens
gtaagtcacctctgagtgagggagctgcacagtggataaggcatttggtgcccagtgtcagaaggagggcagggactctcagtagacacttatctttttgtgtctcaacag

SEQ ID NO: 82 - DNA, 363 bp, Homo sapiens
gtgagtcatgcagagagaacactcctgctgggatgagcatctctgggagccagaggacagtgtttaattgtgatcttattccacttgtcagtggtattgacactgctgactgccttgtcctgtcttcagagtctgtcttccctgagaaggcaaagcacctttctttcttgctgtgccttacattttgctggtcaagcctttcagtttcttttgacagttttttttacttctttcttttttcaatgttgctcttaccaagagtagctcctctgccttccactttacacatgagagctgggcgacgccattcagtcctaaggcttttaccatcacctctcttggtgtttttattgtcatctctaa

SEQ ID NO: 83 - DNA, 434 bp, Homo sapiens
tttaattgattcactaggatatatgctactgaaaggggaatctgcttaaagtgctttctgatatttattattactaaaacttagaatttattaaaaatactgactgtgaaaaattacttgggtcgtttgcctttttaaaaggatttttggcatgtctcattaaaaaaagaaatactagatatcttcagtgaagttacaaatcgaatacacattggctctgaaattctgattgatactgggtcataaaaagttttcccaaatcagacttggaaagtgatcactctcttgttactcttttttccttgtcatgggtgatagccatttgtgtttattggaagatcggtgaattttaaggaacataggcccaaatttgaggaagggccatggtttttgatccctccattctgaccggatctctgcattgtgtctactag

SEQ ID NO: 84 - DNA, 264 bp, Homo sapiens
gtgagctttttcttagaacccgtggagcacctggttgagggtcacagaggaggcgcacagggaaacactcaccaatgggggttgcattgaactgaactcaaaatatgtgataaaactgattttcctgatgtgggcatcccgcagccccctccctgcccatcctggagactgtggcaagtaggttttataatactacgttagagactgaatctttgtcctgaaaaatagtttgaaaggttcatttttcttgttttttcccccaag

SEQ ID NO: 85 - DNA, 175 bp, Homo sapiens
gtgagagagtacaggttacaatagctcatcttcagtttttttcagctttatgtgctgtaacccagcagtttgctgacttgcttaataaaagggcatgtgttcccaaaatgtacatctataccaaggttctgtcaattttattttaaaaacaccatggagacttcttaaagaattc

SEQ ID NO: 86 - DNA, 588 bp, Homo sapiens
gaattcccattctcgaatacattggttttatatgcttacatttatgtgttagttattaaaacatactaatattgtatatctagtcaaaactgaggtagagagaataaatggttgattttgagtttgagtttcatagtccaaaaagctgatatattgcctgtgttcaagagggtctatatcagccctctagatgccagcatctccaaattttacttttttggaatctgtacagtatttgcaatatttttattacaaatttctactctgtggaatttaatttttaaaatacctgcaatacatatatatgttgaatagatgaaaaattatgtagataataatgaatgatacggttctaaaaagacaggttaaaaagtaagttcacttttattttgagcttcagaatcattcagaagccagtcgccacaaacgcagaccaaggctcttggcacatcaaatatgcctatggcttagggttattgacaagtcttatgttgcagtgtatgtggtttatagtcctgccttccacagttgcttgggagagctgtgagtcactgaggcttatgaatgtttacattttgtttgttgcag

SEQ ID NO: 87 - DNA, 78 bp, Homo sapiens
gtaaagacactttgtctatattgcgtttgtccctattagttcagactatctctacccaatcaagcaacgatgctcgtt

SEQ ID NO: 88 - DNA, 376 bp, Homo sapiens
acatgtgccagtactggtgagagcgcaagctttggagtcaaacacaaatgggtttgcatcctggccctaccaattatgagctctgagccatgggcaagtgactaactccctgggcctcagtttctctgtaacatctgtcagacttcatgggtccaggtgaggattaaaggagatcatgtatttacagcacatggcatggtgcttcacataaaataagtatttagtaaatgataactggttccttctctcagaaacttatttctgggcctgccaggggccgccctttttcatggcacaagttgggttcccagggttcagtattcttttaaatagttttctggagatcctccatttgggtattttttcctgctttcag

SEQ ID NO: 89 - DNA, 111 bp, Homo sapiens
gtaagctttgagtgtcaaaacagatttacttctcagggtgtggattcctgccccgacactcccgcccataggtccaagagcagtttgtatcttgaattggtgcttgaattc

SEQ ID NO: 90 - DNA, 2264 bp, Homo sapiens
ctgtatagtaatcattgactaaagccatttgtctgtgttttcttcttgtggttgtatatatcaggtaaaatattttccaaagagccatgtgtcatgtaatactgaaccactttgatattgagacattaatttgtaccctgtgttattatctactagtaataatgtaatactgtagaaatattgctctaattcttttcaaaattgttgcatcccccttagaatgtttctatttccataaggatttaggtatgctattatcccttcttataccctaagatgaagctgtttttgtgctctttgttcatcattggccctcattccaagcactttacgctgtctgtaatgggatctatttttgcactggaatatctgagaattgcaaaactagacaaaagtttcacaacagattttctaagttaaatcattttcattaaaaggaaaaaagaaaaaaaatttttgtatgtcaataacctttatatgaagtattaaaatgcatatttctatgttgtaatataatgagtcacaaaataaagctgtgacagttctgttggtctacagaaatttacttttgtgcatttgtggcaccacctactgttgaagggttataaagccattagaaaagtagaggggaagtgatttggatcaaaaggaaaaactttagaaaagattcaaatgttcccttaatcataaaagagaactgaggggactacttgaaaataaaaggttgttttgtattttcatgttggttaagatactgagtaactggtattaagtgttagaggtttttagataaatattctgcttaatgattatgaagctgcactgagatttctgaaaatgctctgtagctgagcttatttaataaatgttcacttggtataggggaagctacaaaggcagccttcagtgtccttttgtttattcaaccaaaaatataaggacacaatgtagcagttatactgggaaggtgctgggggtggtggcaatggtgagcaggaaggcgaagtagatatggaaacagaaatgatactaatatcggtgattccttccttttttcctgtaataagtgctgtgcagacaacatatgagcagtgctgataaatgtaaatgtatttttcatagctcattaagaatcagtttcagaaagagatgtctgcttattttgctacttgaagaatccctgtcaaacagtccttttgaggaagtacaagaggctgtctctatttgtgacctcaggaatggctgtgacagtgtcgtgagcagtccttttcctgtggcacagatctgaactttgtgtgcagaaaaatcttggcttcaagtgagccaagatgccccctgagcatcagcatcacaacttcatcctcctatcttgaagttcatgttatagtgactttaatgaaatcatagaacactgtttcttcgtgaacaatgacgagggagaggaaaaaactttattgaaaaataaaaaggcaggtaatttagatgaaaatatgttacccatgaggttttgtttttgctttttgtttttgtttttgagaaacagaatctcgctctgtcgtccaggctggagtgcagcggcatgatcttggctcactgcaacctccgcctcccgggttcaagcgattctcctcagcttcccaagtagctggtactacaggcatgcgccaccacaaccagctaatttttgtatttttagtagagatggggtttcactatacgttggccaggctggtctcaaactcctgacctaaggtgatccttctgccttgggctcccaaagtgctgggattacaggcatgagccaccttgcctggccctacccatgagccttgactaaaacattcttctatctgtagaaaagcccaaaagaacttttccagattcaaaaaacttggcactttgtaatggtaatgtttacattaagtaaaaaaaaaaaaaaaaaacccacttagcttcagttttcaagtgtttactgtgttgtcatgcacttcatttaattctcaacacctgccctatgaggtaaaaagtaccattttacatatgagtaaattacagctcagtggataagaaactcgtccaaaggtacaggttcagtcaagtggcagagggttctttttgttgaagttaggtatcagttaaaattgaccttgtaaaatcacatcagcatcaatatacattaatttaacaaatatttattgaactttactgtatgccagatacttctctaggtactagggggtacaatgtagaagaaaatagaattc

SEQ ID NO: 91 - DNA, 9497 bp, Homo sapiens
  misc_feature (6765)..(6765): n is a, c, g, or t
caaacatgtcagctgttactggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctgtatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaagggagaaactggctgcagcagagcgagtacttcgttccaacatggacatcctgaagccaatcctgagaacactaaactctacatctcccttcccgagcaaggagctggccgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatatcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccaccaaacaaatgaagaaacacctaaagctggccaaggacagttctgcagatcgatttctcaacagcttgggaagatttatgacaggactggacaccagaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctccgatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattacgttgtccctgccacactggtcattatcatcttcatctgcttccagcagaagtcctatgtgtcctccaccaatctgcctgtgctagcccttctacttttgctgtatgggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaataagctgaataatatcaatgatatcctgaagtccgtgttcttgatcttcccacatttttgcctgggacgagggctcatcgacatggtgaaaaaccaggcaatggctgatgccctggaaaggtttggggagaatcgctttgtgtcaccattatcttgggacttggtgggacgaaacctcttcgccatggccgtggaaggggtggtgttcttcctcattactgttctgatccagtacagattcttcatcaggcccagacctgtaaatgcaaagctatctcctctgaatgatgaagatgaagatgtgaggcgggaaagacagagaattcttgatggtggaggccagaatgacatcttagaaatcaaggagttgacgaagatatatagaaggaagcggaagcctgctgttgacaggatttgcgtgggcattcctcctggtgagtgctttgggctcctgggagttaatggggctggaaaatcatcaactttcaagatgttaacaggagataccactgttaccagaggagatgctttccttaacagaaatagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagaatgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttccaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatgaagaatcctgttcatacggggtggctgaaagtaaagaggnactagactttcctttgcaccatgtgaagtgttgtggagaaaagagccagaagttgatgtgggaagaagtaaactggatactgtactgatactattcaatgcaatgcaattcaatgcaatgaaaacaaaattccattacaggggcagtgcctttgtagcctatgtcttgtatggctctcaagtgaaagacttgaatttagttttttacctatacctatgtgaaactctattatggaacccaatggacatatgggtttgaactcacactttttttttttttttgttcctgtgtattctcattggggttgcaacaataattcatcaagtaatcatggccagcgattattgatcaaaatcaaaaggtaatgcacatcctcattcactaagccatgccatgcccaggagactggtttcccggtgacacatccattgctggcaatgagtgtgccagagttattagtgccaagtttttcagaaagtttgaagcaccatggtgtgtcatgctcacttttgtgaaagctgctctgctcagagtctatcaacattgaatatcagttgacagaatggtgccatgcgtggctaacatcctgctttgattccctctgataagctgttctggtggcagtaacatgcaacaaaaatgtgggtgtctctaggcacgggaaacttggttccattgttatattgtcctatgcttcgagccatgggtctacagggtcatccttatgagactcttaaatatacttagatcctggtaagaggcaaagaatcaacagccaaactgctggggctgcaagctgctgaagccagggcatgggattaaagagattgtgcgttcaaacctagggaagcctgtgcccatttgtcctgactgtctgctaacatggtacactgcatctcaagatgtttatctgacacaagtgtattatttctggctttttgaattaatctagaaaatgaaaagatggagttgtattttgacaaaaatgtttgtactttttaatgttatttggaattttaagttctatcagtgacttctgaatccttagaatggcctctttgtagaaccctgtggtatagaggagtatggccactgccccactatttttattttcttatgtaagtttgcatatcagtcatgactagtgcctagaaagcaatgtgatggtcaggatctcatgacattatatttgagtttctttcagatcatttaggatactcttaatctcacttcatcaatcaaatattttttgagtgtatgctgtagctgaaagagtatgtacgtacgtataagactagagagatattaagtctcagtacacttcctgtgccatgttattcagctcactggtttacaaatataggttgtcttgtggttgtaggagcccactgtaacaatactgggcagcctttttttttttttttaattgcaacaatgcaaaagccaagaaagtataagggtcacaagtctaaacaatgaattcttcaacagggaaaacagctagcttgaaaacttgctgaaaaacacaacttgtgtttatggcatttagtaccttcaaataattggctttgcagatattggataccccattaaatctgacagtctcaaatttttcatctcttcaatcactagtcaagaaaaatataaaaacaacaaatacttccatatggagcatttttcagagttttctaacccagtcttatttttctagtcagtaaacatttgtaaaaatactgtttcactaatacttactgttaactgtcttgagagaaaagaaaaatatgagagaactattgtttggggaagttcaagtgatctttcaatatcattactaacttcttccactttttccaaaatttgaatattaacgctaaaggtgtaagacttcagatttcaaattaatctttctatattttttaaatttacagaatattatataacccactgctgaaaaagaaaaaaatgattgttttagaagttaaagtcaatattgattttaaatataagtaatgaaggcatatttccaataactagtgatatggcatcgttgcattttacagtatcttcaaaaatacagaatttatagaataatttctcctcatttaatatttttcaaaatcaaagttatggtttcctcattttactaaaatcgtattctaattcttcattatagtaaatctatgagcaactccttacttcggttcctctgatttcaaggccatattttaaaaaatcaaaaggcactgtgaactattttgaagaaaacacaacattttaatacagattgaaaggacctcttctgaagctagaaacaatctatagttatacatcttcattaatactgtgttaccttttaaaatagtaattttttacattttcctgtgtaaacctaattgtggtagaaatttttaccaactctatactcaatcaagcaaaatttctgtatattccctgtggaatgtacctatgtgagtttcagaaattctcaaaatacgtgttcaaaaatttctgcttttgcatctttgggacacctcagaaaacttattaacaactgtgaatatgagaaatacagaagaaaataataagccctctatacataaatgcccagcacaattcattgttaaaaaacaaccaaacctcacactactgtatttcattatctgtactgaaagcaaatgctttgtgactattaaatgttgcacatcattcattcaaaaaaaaaaaaaaaaaaa

SEQ ID NO: 92 - DNA, 2618 bp, Homo sapiens
gcaatgaaaacaaaattccattacaggggcagtgcctttgtagcctatgtcttgtatggctctcaagtgaaagacttgaatttagttttttacctatacctatgtgaaactctattatggaacccaatggacatatgggtttgaactcacactttttttttttttttgttcctgtgtattctcattggggttgcaacaataattcatcaagtaatcatggccagcgattattgatcaaaatcaaaaggtaatgcacatcctcattcactaagccatgccatgcccaggagactggtttcccggtgacacatccattgctggcaatgagtgtgccagagttattagtgccaagtttttcagaaagtttgaagcaccatggtgtgtcatgctcacttttgtgaaagctgctctgctcagagtctatcaacattgaatatcagttgacagaatggtgccatgcgtggctaacatcctgctttgattccctctgataagctgttctggtggcagtaacatgcaacaaaaatgtgggtgtctctaggcacgggaaacttggttccattgttatattgtcctatgcttcgagccatgggtctacagggtcatccttatgagactcttaaatatacttagatcctggtaagaggcaaagaatcaacagccaaactgctggggctgcaagctgctgaagccagggcatgggattaaagagattgtgcgttcaaacctagggaagcctgtgcccatttgtcctgactgtctgctaacatggtacactgcatctcaagatgtttatctgacacaagtgtattatttctggctttttgaattaatctagaaaatgaaaagatggagttgtattttgacaaaaatgtttgtactttttaatgttatttggaattttaagttctatcagtgacttctgaatccttagaatggcctctttgtagaaccctgtggtatagaggagtatggccactgccccactatttttattttcttatgtaagtttgcatatcagtcatgactagtgcctagaaagcaatgtgatggtcaggatctcatgacattatatttgagtttctttcagatcatttaggatactcttaatctcacttcatcaatcaaatattttttgagtgtatgctgtagctgaaagagtatgtacgtacgtataagactagagagatattaagtctcagtacacttcctgtgccatgttattcagctcactggtttacaaatataggttgtcttgtggttgtaggagcccactgtaacaatactgggcagcctttttttttttttttaattgcaacaatgcaaaagccaagaaagtataagggtcacaagtctaaacaatgaattcttcaacagggaaaacagctagcttgaaaacttgctgaaaaacacaacttgtgtttatggcatttagtaccttcaaataattggctttgcagatattggataccccattaaatctgacagtctcaaatttttcatctcttcaatcactagtcaagaaaaatataaaaacaacaaatacttccatatggagcatttttcagagttttctaacccagtcttatttttctagtcagtaaacatttgtaaaaatactgtttcactaatacttactgttaactgtcttgagagaaaagaaaaatatgagagaactattgtttggggaagttcaagtgatctttcaatatcattactaacttcttccactttttccaaaatttgaatattaacgctaaaggtgtaagacttcagatttcaaattaatctttctatattttttaaatttacagaatattatataacccactgctgaaaaagaaaaaaatgattgttttagaagttaaagtcaatattgattttaaatataagtaatgaaggcatatttccaataactagtgatatggcatcgttgcattttacagtatcttcaaaaatacagaatttatagaataatttctcctcatttaatatttttcaaaatcaaagttatggtttcctcattttactaaaatcgtattctaattcttcattatagtaaatctatgagcaactccttacttcggttcctctgatttcaaggccatattttaaaaaatcaaaaggcactgtgaactattttgaagaaaacacaacattttaatacagattgaaaggacctcttctgaagctagaaacaatctatagttatacatcttcattaatactgtgttaccttttaaaatagtaattttttacattttcctgtgtaaacctaattgtggtagaaatttttaccaactctatactcaatcaagcaaaatttctgtatattccctgtggaatgtacctatgtgagtttcagaaattctcaaaatacgtgttcaaaaatttctgcttttgcatctttgggacacctcagaaaacttattaacaactgtgaatatgagaaatacagaagaaaataataagccctctatacataaatgcccagcacaattcattgttaaaaaacaaccaaacctcacactactgtatttcattatctgtactgaaagcaaatgctttgtgactattaaatgttgcacatcattcattcaaaaaaaaaaaaaaaaaaa

SEQ ID NO: 93 - DNA, 302 bp, Homo sapiens
tgtgtcaacctgaacaagctagaacccatagcaacagaagtctggctcatcaacaagtccatggagctgctggagtacagtggcgtgacctcagctcactgcaacctctgcctcctgagttcaagtgattctcgtgcctcagcctcccaagtagctgggattacagctcctgccaccacgcccggggctggtattgtgttcactggaattactccaggcagcattgagctgccccatcatgtcaagtacaagatccgaatggacattgacaatgtggagaggacaaataaaatcaaggatgg

SEQ ID NO: 94 - DNA, 9593 bp, Homo sapiens
  misc_feature (6861)..(6861): n is a, c, g, or t
caaacatgtcagctgttactggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctgtatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaagggagaaactggctgcagcagagcgagtacttcgttccaacatggacatcctgaagccaatcctgagaacactaaactctacatctcccttcccgagcaaggagctggccgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggagtacagtggcgtgacctcagctcactgcaacctctgcctcctgagttcaagtgattctcgtgcctcagcctcccaagtagctgggattacagctcctgccaccacgcccggggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatatcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccaccaaacaaatgaagaaacacctaaagctggccaaggacagttctgcagatcgatttctcaacagcttgggaagatttatgacaggactggacaccagaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctccgatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattacgttgtccctgccacactggtcattatcatcttcatctgcttccagcagaagtcctatgtgtcctccaccaatctgcctgtgctagcccttctacttttgctgtatgggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaataagctgaataatatcaatgatatcctgaagtccgtgttcttgatcttcccacatttttgcctgggacgagggctcatcgacatggtgaaaaaccaggcaatggctgatgccctggaaaggtttggggagaatcgctttgtgtcaccattatcttgggacttggtgggacgaaacctcttcgccatggccgtggaaggggtggtgttcttcctcattactgttctgatccagtacagattcttcatcaggcccagacctgtaaatgcaaagctatctcctctgaatgatgaagatgaagatgtgaggcgggaaagacagagaattcttgatggtggaggccagaatgacatcttagaaatcaaggagttgacgaagatatatagaaggaagcggaagcctgctgttgacaggatttgcgtgggcattcctcctggtgagtgctttgggctcctgggagttaatggggctggaaaatcatcaactttcaagatgttaacaggagataccactgttaccagaggagatgctttccttaacagaaatagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagaatgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttccaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatgaagaatcctgttcatacggggtggctgaaagtaaagaggnactagactttcctttgcaccatgtgaagtgttgtggagaaaagagccagaagttgatgtgggaagaagtaaactggatactgtactgatactattcaatgcaatgcaattcaatgcaatgaaaacaaaattccattacaggggcagtgcctttgtagcctatgtcttgtatggctctcaagtgaaagacttgaatttagttttttacctatacctatgtgaaactctattatggaacccaatggacatatgggtttgaactcacactttttttttttttttgttcctgtgtattctcattggggttgcaacaataattcatcaagtaatcatggccagcgattattgatcaaaatcaaaaggtaatgcacatcctcattcactaagccatgccatgcccaggagactggtttcccggtgacacatccattgctggcaatgagtgtgccagagttattagtgccaagtttttcagaaagtttgaagcaccatggtgtgtcatgctcacttttgtgaaagctgctctgctcagagtctatcaacattgaatatcagttgacagaatggtgccatgcgtggctaacatcctgctttgattccctctgataagctgttctggtggcagtaacatgcaacaaaaatgtgggtgtctctaggcacgggaaacttggttccattgttatattgtcctatgcttcgagccatgggtctacagggtcatccttatgagactcttaaatatacttagatcctggtaagaggcaaagaatcaacagccaaactgctggggctgcaagctgctgaagccagggcatgggattaaagagattgtgcgttcaaacctagggaagcctgtgcccatttgtcctgactgtctgctaacatggtacactgcatctcaagatgtttatctgacacaagtgtattatttctggctttttgaattaatctagaaaatgaaaagatggagttgtattttgacaaaaatgtttgtactttttaatgttatttggaattttaagttctatcagtgacttctgaatccttagaatggcctctttgtagaaccctgtggtatagaggagtatggccactgccccactatttttattttcttatgtaagtttgcatatcagtcatgactagtgcctagaaagcaatgtgatggtcaggatctcatgacattatatttgagtttctttcagatcatttaggatactcttaatctcacttcatcaatcaaatattttttgagtgtatgctgtagctgaaagagtatgtacgtacgtataagactagagagatattaagtctcagtacacttcctgtgccatgttattcagctcactggtttacaaatataggttgtcttgtggttgtaggagcccactgtaacaatactgggcagcctttttttttttttttaattgcaacaatgcaaaagccaagaaagtataagggtcacaagtctaaacaatgaattcttcaacagggaaaacagctagcttgaaaacttgctgaaaaacacaacttgtgtttatggcatttagtaccttcaaataattggctttgcagatattggataccccattaaatctgacagtctcaaatttttcatctcttcaatcactagtcaagaaaaatataaaaacaacaaatacttccatatggagcatttttcagagttttctaacccagtcttatttttctagtcagtaaacatttgtaaaaatactgtttcactaatacttactgttaactgtcttgagagaaaagaaaaatatgagagaactattgtttggggaagttcaagtgatctttcaatatcattactaacttcttccactttttccaaaatttgaatattaacgctaaaggtgtaagacttcagatttcaaattaatctttctatattttttaaatttacagaatattatataacccactgctgaaaaagaaaaaaatgattgttttagaagttaaagtcaatattgattttaaatataagtaatgaaggcatatttccaataactagtgatatggcatcgttgcattttacagtatcttcaaaaatacagaatttatagaataatttctcctcatttaatatttttcaaaatcaaagttatggtttcctcattttactaaaatcgtattctaattcttcattatagtaaatctatgagcaactccttacttcggttcctctgatttcaaggccatattttaaaaaatcaaaaggcactgtgaactattttgaagaaaacacaacattttaatacagattgaaaggacctcttctgaagctagaaacaatctatagttatacatcttcattaatactgtgttaccttttaaaatagtaattttttacattttcctgtgtaaacctaattgtggtagaaatttttaccaactctatactcaatcaagcaaaatttctgtatattccctgtggaatgtacctatgtgagtttcagaaattctcaaaatacgtgttcaaaaatttctgcttttgcatctttgggacacctcagaaaacttattaacaactgtgaatatgagaaatacagaagaaaataataagccctctatacataaatgcccagcacaattcattgttaaaaaacaaccaaacctcacactactgtatttcattatctgtactgaaagcaaatgctttgtgactattaaatgttgcacatcattcattcaaaaaaaaaaaaaaaaaaa

SEQ ID NO: 95 - DNA, 173 bp, Homo sapiens
ctgggaccctggtcctcgagctgacccctttgaggacatgcggtacgtctgggggggcttcgcctacttgcaggatgtggtggagcaggcaatcatcagggtgctacgggcaccgagaagaaaactggtgtctatatgcaacagatgccctatccctgttacgttgatgacat

SEQ ID NO: 96 - DNA, 9495 bp, Homo sapiens
  misc_feature (6763)..(6763): n is a, c, g, or t
caaacatgtcagctgttactggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctgtatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaagggagaaactggctgcagcagagcgagtacttcgttccaacatggacatcctgaagccaatcctgagaacactaaactctacatctcccttcccgagcaaggagctggccgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctcgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatatcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccaccaaacaaatgaagaaacacctaaagctggccaaggacagttctgcagatcgatttctcaacagcttgggaagatttatgacaggactggacaccagaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctccgatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattacgttgtccctgccacactggtcattatcatcttcatctgcttccagcagaagtcctatgtgtcctccaccaatctgcctgtgctagcccttctacttttgctgtatgggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaataagctgaataatatcaatgatatcctgaagtccgtgttcttgatcttcccacatttttgcctgggacgagggctcatcgacatggtgaaaaaccaggcaatggctgatgccctggaaaggtttggggagaatcgctttgtgtcaccattatcttgggacttggtgggacgaaacctcttcgccatggccgtggaaggggtggtgttcttcctcattactgttctgatccagtacagattcttcatcaggcccagacctgtaaatgcaaagctatctcctctgaatgatgaagatgaagatgtgaggcgggaaagacagagaattcttgatggtggaggccagaatgacatcttagaaatcaaggagttgacgaagatatatagaaggaagcggaagcctgctgttgacaggatttgcgtgggcattcctcctggtgagtgctttgggctcctgggagttaatggggctggaaaatcatcaactttcaagatgttaacaggagataccactgttaccagaggagatgctttccttaacagaaatagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagaatgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttccaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatgaagaatcctgttcatacggggtggctgaaagtaaagaggnactagactttcctttgcaccatgtgaagtgttgtggagaaaagagccagaagttgatgtgggaagaagtaaactggatactgtactgatactattcaatgcaatgcaattcaatgcaatgaaaacaaaattccattacaggggcagtgcctttgtagcctatgtcttgtatggctctcaagtgaaagacttgaatttagttttttacctatacctatgtgaaactctattatggaacccaatggacatatgggtttgaactcacactttttttttttttttgttcctgtgtattctcattggggttgcaacaataattcatcaagtaatcatggccagcgattattgatcaaaatcaaaaggtaatgcacatcctcattcactaagccatgccatgcccaggagactggtttcccggtgacacatccattgctggcaatgagtgtgccagagttattagtgccaagtttttcagaaagtttgaagcaccatggtgtgtcatgctcacttttgtgaaagctgctctgctcagagtctatcaacattgaatatcagttgacagaatggtgccatgcgtggctaacatcctgctttgattccctctgataagctgttctggtggcagtaacatgcaacaaaaatgtgggtgtctctaggcacgggaaacttggttccattgttatattgtcctatgcttcgagccatgggtctacagggtcatccttatgagactcttaaatatacttagatcctggtaagaggcaaagaatcaacagccaaactgctggggctgcaagctgctgaagccagggcatgggattaaagagattgtgcgttcaaacctagggaagcctgtgcccatttgtcctgactgtctgctaacatggtacactgcatctcaagatgtttatctgacacaagtgtattatttctggctttttgaattaatctagaaaatgaaaagatggagttgtattttgacaaaaatgtttgtactttttaatgttatttggaattttaagttctatcagtgacttctgaatccttagaatggcctctttgtagaaccctgtggtatagaggagtatggccactgccccactatttttattttcttatgtaagtttgcatatcagtcatgactagtgcctagaaagcaatgtgatggtcaggatctcatgacattatatttgagtttctttcagatcatttaggatactcttaatctcacttcatcaatcaaatattttttgagtgtatgctgtagctgaaagagtatgtacgtacgtataagactagagagatattaagtctcagtacacttcctgtgccatgttattcagctcactggtttacaaatataggttgtcttgtggttgtaggagcccactgtaacaatactgggcagcctttttttttttttttaattgcaacaatgcaaaagccaagaaagtataagggtcacaagtctaaacaatgaattcttcaacagggaaaacagctagcttgaaaacttgctgaaaaacacaacttgtgtttatggcatttagtaccttcaaataattggctttgcagatattggataccccattaaatctgacagtctcaaatttttcatctcttcaatcactagtcaagaaaaatataaaaacaacaaatacttccatatggagcatttttcagagttttctaacccagtcttatttttctagtcagtaaacatttgtaaaaatactgtttcactaatacttactgttaactgtcttgagagaaaagaaaaatatgagagaactattgtttggggaagttcaagtgatctttcaatatcattactaacttcttccactttttccaaaatttgaatattaacgctaaaggtgtaagacttcagatttcaaattaatctttctatattttttaaatttacagaatattatataacccactgctgaaaaagaaaaaaatgattgttttagaagttaaagtcaatattgattttaaatataagtaatgaaggcatatttccaataactagtgatatggcatcgttgcattttacagtatcttcaaaaatacagaatttatagaataatttctcctcatttaatatttttcaaaatcaaagttatggtttcctcattttactaaaatcgtattctaattcttcattatagtaaatctatgagcaactccttacttcggttcctctgatttcaaggccatattttaaaaaatcaaaaggcactgtgaactattttgaagaaaacacaacattttaatacagattgaaaggacctcttctgaagctagaaacaatctatagttatacatcttcattaatactgtgttaccttttaaaatagtaattttttacattttcctgtgtaaacctaattgtggtagaaatttttaccaactctatactcaatcaagcaaaatttctgtatattccctgtggaatgtacctatgtgagtttcagaaattctcaaaatacgtgttcaaaaatttctgcttttgcatctttgggacacctcagaaaacttattaacaactgtgaatatgagaaatacagaagaaaataataagccctctatacataaatgcccagcacaattcattgttaaaaaacaaccaaacctcacactactgtatttcattatctgtactgaaagcaaatgctttgtgactattaaatgttgcacatcattcattcaaaaaaaaaaaaaaaaaaa

SEQ ID NO: 97 - DNA, 41 bp, Homo sapiens
tgaaggctgttcttctatcagtgtgtcaacctgaacaagct

SEQ ID NO: 98 - DNA, 41 bp, Homo sapiens
tgaaggctgttcttctatcaatgtgtcaacctgaacaagct

SEQ ID NO: 99 - DNA, 41 bp, Homo sapiens
agttacctgcaagccactgtttttaaccagtttatactgtg

SEQ ID NO: 100 - DNA, 41 bp, Homo sapiens
agttacctgcaagccactgtatttaaccagtttatactgtg

SEQ ID NO: 101 - DNA, 41 bp, Homo sapiens
caggctcagaggccttggcccatcaccctggctcacgtgtg

SEQ ID NO: 102 - DNA, 41 bp, Homo sapiens
caggctcagaggccttggcctatcaccctggctcacgtgtg

SEQ ID NO: 103 - DNA, 41 bp, Homo sapiens
atgggcctggacaacagcatcctctggtttagctggttcat

SEQ ID NO: 104 - DNA, 41 bp, Homo sapiens
atgggcctggacaacagcatactctggtttagctggttcat

SEQ ID NO: 105 - DNA, 41 bp, Homo sapiens
aagggaggagaagaagaaaaaaaatccaagcctctggtaga

SEQ ID NO: 106 - DNA, 41 bp, Homo sapiens
aagggaggagaagaagaaaagaaatccaagcctctggtaga

SEQ ID NO: 107 - DNA, 41 bp, Homo sapiens
tcttccctttgcagagacacgccctgccaggcaggggagga

SEQ ID NO: 108 - DNA, 41 bp, Homo sapiens
tcttccctttgcagagacacaccctgccaggcaggggagga

SEQ ID NO: 109 - DNA, 23 bp, Homo sapiens
ccttgcctcctagtgtaggattt

SEQ ID NO: 110 - DNA, 24 bp, Homo sapiens
aatataataggtgctctggacctc

SEQ ID NO: 111 - DNA, 25 bp, Homo sapiens
atacaaaaatagaaaaaggggcttg

SEQ ID NO: 112 - DNA, 25 bp, Homo sapiens
atggatgagaaggaaagaggtttac

SEQ ID NO: 113 - DNA, 25 bp, Homo sapiens
cattggatcatacgtacatttcaga

SEQ ID NO: 114 - DNA, 25 bp, Homo sapiens
tcactttccccaactataaatggat

SEQ ID NO: 115 - DNA, 25 bp, Homo sapiens
gtagatcatacaagtgagtgcttgg

SEQ ID NO: 116 - DNA, 25 bp, Homo sapiens
ctgttctcaacttgctgcttttatt

SEQ ID NO: 117 - DNA, 23 bp, Homo sapiens
gcaaattcaaatttctccaggta

SEQ ID NO: 118 - DNA, 23 bp, Homo sapiens
gcacaaagaaaggacatcagcta

SEQ ID NO: 119 - DNA, 23 bp, Homo sapiens
cagtgcttacccctgctaatatc

SEQ ID NO: 120 - DNA, 23 bp, Homo sapiens
gagatggagaaatcattcacagc

SEQ ID NO: 121 - DNA, 23 bp, Homo sapiens
acatgtggaatgacctaaacacc

SEQ ID NO: 122 - DNA, 23 bp, Homo sapiens
cttaggacatttggccttgctat

SEQ ID NO: 123 - DNA, 24 bp, Homo sapiens
catttctgttttaagagcctgtca

SEQ ID NO: 124 - DNA, 23 bp, Homo sapiens
aatgtggcatgcagttgataaat

SEQ ID NO: 125 - DNA, 23 bp, Homo sapiens
gtttgtggttgttacggaatgat

SEQ ID NO: 126 - DNA, 23 bp, Homo sapiens
cctcccaacatgatatctcactc

SEQ ID NO: 127 - DNA, 23 bp, Homo sapiens
gtctgggacctgtagtcaggttt

SEQ ID NO: 128 - DNA, 23 bp, Homo sapiens
ccaatagacagaatcaggccata

SEQ ID NO: 129 - DNA, 23 bp, Homo sapiens
tgccaacatttattagaggaagc

SEQ ID NO: 130 - DNA, 23 bp, Homo sapiens
atccgtttaacctgccaactact

SEQ ID NO: 131 - DNA, 23 bp, Homo sapiens
tctcaggagccgtttatttaatg

SEQ ID NO: 132 - DNA, 23 bp, Homo sapiens
gccaactttaccatgagttgaaa

SEQ ID NO: 133 - DNA, 23 bp, Homo sapiens
tctgatcatagtgttttgccttg

SEQ ID NO: 134 - DNA, 23 bp, Homo sapiens
tgttcccctacaatgagattcac

SEQ ID NO: 135 - DNA, 22 bp, Homo sapiens
gggtgaacagatgtttttcctt

SEQ ID NO: 136 - DNA, 23 bp, Homo sapiens
tagctggaacatttcctgatgat

SEQ ID NO: 137 - DNA, 23 bp, Homo sapiens
ccctttcttgtctgataatggtg

SEQ ID NO: 138 - DNA, 23 bp, Homo sapiens
cacaattaaacactgtcctctgg

SEQ ID NO: 139 - protein, 2201 aa, Homo sapiens [sequence omitted]

SEQ ID NO: 140 - protein, 2233 aa, Homo sapiens
MPSAGTLPWVQGIICNANNPCFRYPTPGEAPGVVGNFNKSIVARLFSDARRLLLYSQKDTSMKDMRKVLRTLQQIKKSSSNLKLQDFLVDNETFSGFLYHNLSLPKSTVDKMLRADVILHKVFLQGYQLHLTSLCNGSKSEEMIQLGDQEVSELCGLPREKLAAAERVLRSNMDILKPILRTLNSTSPFPSKELAEATKTLLHSLGTLAQELFSMRSWSDMRQEVMFLTNVNSSSSSTQIYQAVSRIVCGHPEGGGLKIKSLNWYEDNNYKALFGGNGTEEDAETFYDNSTTPYCNDLMKNLESSPLSRIIWKALKPLLVGKILYTPDTPATRQVMAEVNKTFQELAVFHDLEGMWEELSPKIWTFMENSQEMDLVRMLLDSRDNDHFWEQQLDGLDWTAQDIVAFLAKHPEDVQSSNGSVYTWREAFNETNQAIRTISRFMECVNLNKLEPIATEVWLINKSMELLEYSGVTSAHCNLCLLSSSDSRASASQVAGITAPATTPGAGIVFTGITPGSIELPHHVKYKIRMDIDNVERTNKIKDGYWDPGPRADPFEDMRYVWGGFAYLQDVVEQAIIRVLTGTEKKTGVYMQQMPYPCYVDDIFLRVMSRSMPLFMTLAWIYSVAVIIKGIVYEKEARLKETMRIMGLDNSILWFSWFISSLIPLLVSAGLLVVILKLGNLLPYSDPSVVFVFLSVFAVVTILQCFLISTLFSRANLAAACGGIIYFTLYLPYVLCVAWQDYVGFTLKIFASLLSPVAFGFGCEYFALFEEQGIGVQWDNLFESPVEEDGFNLTTSVSMMLFDTFLYGVMTWYIEAVFPGQYGIPRPWYFPCTKSYWFGEESDEKSHPGSNQKRISEICMEEEPTHLKLGVSIQNLVKVYRDGMKVAVDGLALNFYEGQITSFLGHNGAGKTTTMSILTGLFPPTSGTAYILGKDIRSEMSTIRQNLGVCPQHNVLFDMLTVEEHIWFYARLKGLSEKHVKAEMEQMALDVGLPSSKLKSKTSQLSGGMQRKLSVALAFVGGSKVVILDEPTAGVDPYSRRGIWELLLKYRQGRTIILSTHHMDEADVLGDRIAIISHGKLCCVGSSLFLKNQLGTGYYLTLVKKDVESSLSSCRNSSSTVSYLKKEDSVSQSSSDAGLGSDHESDTLTIDVSAISNLIRKHVSEARLVEDIGHELTYVLPYEAAKEGAFVELFHEIDDRLSDLGISSYGISETTLEEIFLKVAEESGVDAETSDGTLPARRNRRAFGDKQSCLRPFTEDDAADPNDSDIDPESRETDLLSGMDGKGSYQVKGWKLTQQQFVALLWKRLLIARRSRKGFFAQIVLPAVFVCIALVFSLIVPPFGKYPSLELQPWMYNEQYTFVSNDAPEDTGTLELLNALTKDPGFGTRCMEGNPIPDTPCQAGEEEWTTAPVPQTIMDLFQNGNWTMQNPSPACQCSSDKIKKMLPVCPPGAGGLPPPQRKQNTADILQDLTGRNISDYLVKTYVQIIAKSLKNKIWVNEFRYGGFSLGVSNTQALPPSQEVNDATKQMKKHLKLAKDSSADRFLNSLGRFMTGLDTRNNVKVWFNNKGWHAISSFLNVINNAILRANLQKGENPSHYGITAFNHPLNLTKQQLSEVAPMTTSVDVLVSICVIFAMSFVPASFVVFLIQERVSKAKHLQFISGVKPVIYWLSNFVWDMCNYVVPATLVIIIFICFQQKSYVSSTNLPVLALLLLLYGWSITPLMYPASFVFKIPSTAYVVLTSVNLFIGINGSVATFVLELFTDNKLNNINDILKSVFLIFPHFCLGRGLIDMVKNQAMADALERFGENRFVSPLSWDLVGRNLFAMAVEGVVFFLITVLIQYRFFIRPRPVNAKLSPLNDEDEDVRRERQRILDGGGQNDILEIKELTKIYRRKRKPAVDRICVGIPPGECFGLLGVNGAGKSSTFKMLTGDTTVTRGDAFLNRNSILSNIHEVHQNMGYCPQFDAITELLTGREHVEFFALLRGVPEKEVGKVGEWAIRKLGLVKYGEKYAGNYSGGNKRKLSTAMALIGGPPVVFLDEPTTGMDPKARRFLWNCALSVVKEGRSVVLTSHSMEECEALCTRMAIMVNGRFRCLGSVQHLKNRFGDGYTIVVRIAGSNPDLKPVQDFFGLAFPGSVPKEKHRNMLQYQLPSSLSSLARIFSILSQSKKRLHIEDYSVSQTTLDQVFVNFAKDQSDDDHLKDLSLHKNQTVVDVAVLTSFLQDEKVKESYV

SEQ ID NO: 141 - protein, 574 aa, Homo sapiens
MPSAGTLPWVQGIICNANNPCFRYPTPGEAPGVVGNFNKSIVARLFSDARRLLLYSQKDTSMKDMRKVLRTLQQIKKSSSNLKLQDFLVDNETFSGFLYHNLSLPKSTVDKMLRADVILHKVFLQGYQLHLTSLCNGSKSEEMIQLGDQEVSELCGLPREKLAAAERVLRSNMDILKPILRTLNSTSPFPSKELAEATKTLLHSLGTLAQELFSMRSWSDMRQEVMFLTNVNSSSSSTQIYQAVSRIVCGHPEGGGLKIKSLNWYEDNNYKALFGGNGTEEDAETFYDNSTTPYCNDLMKNLESSPLSRIIWKALKPLLVGKILYTPDTPATRQVMAEVNKTFQELAVFHDLEGMWEELSPKIWTFMENSQEMDLVRMLLDSRDNDHFWEQQLDGLDWTAQDIVAFLAKHPEDVQSSNGSVYTWREAFNETNQAIRTISRFMECVNLNKLEPIATEVWLINKSMELLDERKFWAGIVFTGITPGSIELPHHVKYKIRMDIDNVERTNKIKDGYWDPGPRADPFEDMRYVWGGFAYLQDVVEQAIIRVLRAPRRKLVSICNRCPIPVTLMTSFCG

SEQ ID NO: 142 - DNA, 20 bp, Artificial
  Oligonucleotide Primer
aaaccagacagtagtggacg

SEQ ID NO: 143 - DNA, 20 bp, Artificial
  Oligonucleotide Primer
gttactgccaccagaacagc

SEQ ID NO: 144 - DNA, 20 bp, Artificial
  Oligonucleotide Primer
tgataagctgttctggtggc

SEQ ID NO: 145 - DNA, 20 bp, Artificial
  Oligonucleotide Primer
cttggcttttgcattgttgc

SEQ ID NO: 146 - DNA, 20 bp, Artificial
  Oligonucleotide Primer
caatgcaaaagccaagaaag

SEQ ID NO: 147 - DNA, 19 bp, Artificial
  Oligonucleotide Primer
tgcaacgatgccatatcac

SEQ ID NO: 148 - DNA, 22 bp, Artificial
  Oligonucleotide Primer
caactccttacttcggttcctc

SEQ ID NO: 149 - DNA, 21 bp, Artificial
  Oligonucleotide Primer
gttttctgaggtgtcccaaag

SEQ ID NO: 150 - DNA, 6 bp, Artificial
  Polyadenylation Sequence
attaaa

SEQ ID NO: 151 - DNA, 14 bp, Artificial
  DNA Oligonucleotide
tgagaggaagttct

SEQ ID NO: 152 - protein, 38 aa, Artificial
  Fragment of mutated ABC1 polypeptide
EYSGVTSAHCNLCLLSSSDSRASASQVAGITAPATTPG

SEQ ID NO: 153 - protein, 26 aa, Artificial
  Fragment of mutated ABC1 polypeptide
RAPRRKLVSICNRCPIPVTLMTSFCG

SEQ ID NO: 154 - protein, 6 aa, Artificial
  Fragment of ABC1 polypeptide
DERKFW

SEQ ID NO: 155 - DNA, 20 bp, Artificial
  Oligonucleotide Primer
ctacccaccctatgaacaac

SEQ ID NO: 156 - DNA, 20 bp, Artificial
  Oligonucleotide Primer
gccaccccgtatgaacaggg

SEQ ID NO: 157 - DNA, 47 bp, Artificial
  Multiple Cloning olignucleotide sequence
cggccgcggcgcgcccggaccgcctaggatttaaatcgcggcccgcg

SEQ ID NO: 158 - DNA, 69 bp, Artificial
  Multiple Cloning Oligonucleotide Sequence
gctctagaattcggcctccgtggccgtttaaacgctagcgcccgggcttaattaagtcgactctagagc

SEQ ID NO: 159 - protein, 27 aa, Artificial
  Synthetic Peptide derived from ABC1 polypeptide
LHKNQTVVDVAVLTSFLQDEKVKESYV